SEQUENCE LISTING (61 sequences)

SEQ ID NO: 1 - DNA, 1356 bp, Artificial Sequence
  Synthetic polynucleotide
caggtccagctgcagcagagtggggccgaactggcaagacccggagcaagcgtcaaaatgtcatgtaaagcaagcggttatactttcactaggagcaccatgcactgggtgaaacagaggcccggccagggactggagtggatcgggtacattaacccttccagcgcttacaccaactataatcagaagttcaaagacaaggccaccctgacagctgataagtctagttcaacagcatatatgcagctgtccagcctgacttctgaagacagtgcagtgtactattgcgcctccccacaggtccactacgattacaatggttttccttactgggggcagggcacactggtgactgtctccgccgctagcacaaagggccctagtgtgtttcctctggctccctcttccaaatccacttctggtggcactgctgctctgggatgcctggtgaaggattactttcctgaacctgtgactgtctcatggaactctggtgctctgacttctggtgtccacactttccctgctgtgctgcagtctagtggactgtactctctgtcatctgtggtcactgtgccctcttcatctctgggaacccagacctacatttgtaatgtgaaccacaaaccatccaacactaaagtggacaaaaaagtggaacccaaatcctgtgacaaaacccacacctgcccaccttgtcctgcccctgaactgctgggaggaccttctgtgtttctgttcccccccaaaccaaaggataccctgatgatctctagaacccctgaggtgacatgtgtggtggtggatgtgtctcatgaggaccctgaggtcaaattcaactggtacgtggatggagtggaagtccacaatgccaaaaccaagcctagagaggaacagtacaattcaacctacagagttgtcagtgtgctgactgtgctgcatcaggattggctgaatggcaaggaatacaagtgtaaagtctcaaacaaggccctgcctgctccaattgagaaaacaatctcaaaggccaagggacagcctagggaaccccaggtctacaccctgccaccttcaagagaggaaatgaccaaaaaccaggtgtccctgacatgcctggtcaaaggcttctacccttctgacattgctgtggagtgggagtcaaatggacagcctgagaacaactacaaaacaaccccccctgtgctggattctgatggctctttctttctgtactccaaactgactgtggacaagtctagatggcagcaggggaatgtcttttcttgctctgtcatgcatgaggctctgcataaccactacactcagaaatccctgtctctgtctcccgggaaatga

SEQ ID NO: 2 - protein, 451 aa, Artificial Sequence
  Synthetic polypeptide
QVQLQQSGAELARPGASVKMSCKASGYTFTRSTMHWVKQRPGQGLEWIGYINPSSAYTNYNQKFKDKATLTADKSSSTAYMQLSSLTSEDSAVYYCASPQVHYDYNGFPYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - DNA, 642 bp, Artificial Sequence
  Synthetic polynucleotide
caggtggtgctgacccagagccctgctattatgtccgcattccccggtgaaaaagtgactatgacttgttccgcttcttcctccgtctcctacatgaactggtatcagcagaagtcaggaacatctcccaaaaggtggatctacgactccagcaagctggcatccggcgtgcctgcacgattctcaggctccggaagcgggacctcttatagtctgacaatttctagtatggagactgaagatgccgctacctactattgccagcagtggtcaagaaaccctccaacattcgggggggggactaaactgcagattactcgtacggtcgcggcgccttctgtgttcattttccccccatctgatgaacagctgaaatctggcactgcttctgtggtctgtctgctgaacaacttctaccctagagaggccaaagtccagtggaaagtggacaatgctctgcagagtgggaattcccaggaatctgtcactgagcaggactctaaggatagcacatactccctgtcctctactctgacactgagcaaggctgattacgagaaacacaaagtgtacgcctgtgaagtcacacatcaggggctgtctagtcctgtgaccaaatccttcaataggggagagtgctga

SEQ ID NO: 4 - protein, 213 aa, Artificial Sequence
  Synthetic polypeptide
QVVLTQSPAIMSAFPGEKVTMTCSASSSVSYMNWYQQKSGTSPKRWIYDSSKLASGVPARFSGSGSGTSYSLTISSMETEDAATYYCQQWSRNPPTFGGGTKLQITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - DNA, 1353 bp, Artificial Sequence
  Synthetic polynucleotide
caggtgcagctccagcagagtggcgcagagctggtgaagcccggagcctcagtcaagatgtcctgcaaggccttcggctacacttttaccacatatcctatcgagtggatgaagcagaaccacgggaaaagcctggaatggattggtaacttccatccatacaatgacgataccaagtataatgagaagtttaaaggcaaggcaaaactgacagtggagaaatccagcactaccgtctacctggaactgtccaggctgacatctgacgatagtgccgtgtactattgtgctcgggaaaactacggaagccacggcggattcgtctattgggggcagggtacactggtgactgtctctgccgctagcacaaagggccctagtgtgtttcctctggctccctcttccaaatccacttctggtggcactgctgctctgggatgcctggtgaaggattactttcctgaacctgtgactgtctcatggaactctggtgctctgacttctggtgtccacactttccctgctgtgctgcagtctagtggactgtactctctgtcatctgtggtcactgtgccctcttcatctctgggaacccagacctacatttgtaatgtgaaccacaaaccatccaacactaaagtggacaaaaaagtggaacccaaatcctgtgacaaaacccacacctgcccaccttgtcctgcccctgaactgctgggaggaccttctgtgtttctgttcccccccaaaccaaaggataccctgatgatctctagaacccctgaggtgacatgtgtggtggtggctgtgtctcatgaggaccctgaggtcaaattcaactggtacgtggatggagtggaagtccacaatgccaaaaccaagcctagagaggaacagtacgcttcaacctacagagttgtcagtgtgctgactgtgctgcatcaggattggctgaatggcaaggaatacaagtgtaaagtctcaaacaaggccctggctgctccaattgagaaaacaatctcaaaggccaagggacagcctagggaaccccaggtctacaccctgccaccttcaagagaggaaatgaccaaaaaccaggtgtccctgacatgcctggtcaaaggcttctacccttctgacattgctgtggagtgggagtcaaatggacagcctgagaacaactacaaaacaaccccccctgtgctggattctgatggctctttctttctgtactccaaactgactgtggacaagtctagatggcagcaggggaatgtcttttcttgctctgtcatgcatgaggctctgcataaccactacactcagaaatccctgtctctgtctcccgggaaatga

SEQ ID NO: 6 - protein, 450 aa, Artificial Sequence
  Synthetic polypeptide
QVQLQQSGAELVKPGASVKMSCKAFGYTFTTYPIEWMKQNHGKSLEWIGNFHPYNDDTKYNEKFKGKAKLTVEKSSTTVYLELSRLTSDDSAVYYCARENYGSHGGFVYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - DNA, 648 bp, Artificial Sequence
  Synthetic polynucleotide
gagaacgtgctgacccagtcccccgcaatcatgtctgccagtcctggagaaaaggtcaccatgacatgcagggcatccagctctgtcatcagttcatacctgcactggtatcagcagaagagcggagcttctccaaaactgtggatctactcaacctccaacctggcaagcggggtgcccgaccggttcagcggctctggaagtgggacttcatatagtctgaccatctcgtcggtcgaggccgaagatgccgctacatactattgtcagcagtacaatggctatcccctgacatttggtgctggtaccaaactcgagattaagcgtacggtcgcggcgccttctgtgttcattttccccccatctgatgaacagctgaaatctggcactgcttctgtggtctgtctgctgaacaacttctaccctagagaggccaaagtccagtggaaagtggacaatgctctgcagagtgggaattcccaggaatctgtcactgagcaggactctaaggatagcacatactccctgtcctctactctgacactgagcaaggctgattacgagaaacacaaagtgtacgcctgtgaagtcacacatcaggggctgtctagtcctgtgaccaaatccttcaataggggagagtgctga

SEQ ID NO: 8 - protein, 215 aa, Artificial Sequence
  Synthetic polypeptide
ENVLTQSPAIMSASPGEKVTMTCRASSSVISSYLHWYQQKSGASPKLWIYSTSNLASGVPDRFSGSGSGTSYSLTISSVEAEDAATYYCQQYNGYPLTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - DNA, 1353 bp, Artificial Sequence
  Synthetic polynucleotide
gaggttcagctggtggagtctggcggtggcctggtgcagccagggggctcactccgtttgtcctgtgcagcttctggcttcaacattaaagacacctatatacactgggtgcgtcaggccccgggtaagggcctggaatgggttgcaaggatttatcctacgaatggttatactagatatgccgatagcgtcaagggccgtttcactataagcgcagacacatccaaaaacacagcctacctgcagatgaacagcctgcgtgctgaggacactgccgtctattattgttctagatggggaggggacggcttctatgctatggactactggggtcaaggaaccctggtcaccgtctcctcggctagcacaaagggccctagtgtgtttcctctggctccctcttccaaatccacttctggtggcactgctgctctgggatgcctggtgaaggattactttcctgaacctgtgactgtctcatggaactctggtgctctgacttctggtgtccacactttccctgctgtgctgcagtctagtggactgtactctctgtcatctgtggtcactgtgccctcttcatctctgggaacccagacctacatttgtaatgtgaaccacaaaccatccaacactaaagtggacaaaaaagtggaacccaaatcctgtgacaaaacccacacctgcccaccttgtcctgcccctgaactgctgggaggaccttctgtgtttctgttcccccccaaaccaaaggataccctgatgatctctagaacccctgaggtgacatgtgtggtggtggctgtgtctcatgaggaccctgaggtcaaattcaactggtacgtggatggagtggaagtccacaatgccaaaaccaagcctagagaggaacagtacgcttcaacctacagagttgtcagtgtgctgactgtgctgcatcaggattggctgaatggcaaggaatacaagtgtaaagtctcaaacaaggccctggctgctccaattgagaaaacaatctcaaaggccaagggacagcctagggaaccccaggtctacaccctgccaccttcaagagaggaaatgaccaaaaaccaggtgtccctgacatgcctggtcaaaggcttctacccttctgacattgctgtggagtgggagtcaaatggacagcctgagaacaactacaaaacaaccccccctgtgctggattctgatggctctttctttctgtactccaaactgactgtggacaagtctagatggcagcaggggaatgtcttttcttgctctgtcatgcatgaggctctgcataaccactacactcagaaatccctgtctctgtctcccgggaaatga

SEQ ID NO: 10 - protein, 450 aa, Artificial Sequence
  Synthetic polypeptide
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - DNA, 645 bp, Artificial Sequence
  Synthetic polynucleotide
gatatccagatgacccagtccccgagctccctgtccgcctctgtgggcgatagggtcaccatcacctgccgtgccagtcaggatgtgaatactgctgtagcctggtatcaacagaaaccaggaaaagctccgaaactactgatttactcggcatccttcctctactctggagtcccttctcgcttctctgggtccagatctgggacggatttcactctgaccatcagcagtctgcagccggaagacttcgcaacttattactgtcagcaacattatactactcctcccacgttcggacaggggaccaaggtggagatcaaacgtacggtcgcggcgccttctgtgttcattttccccccatctgatgaacagctgaaatctggcactgcttctgtggtctgtctgctgaacaacttctaccctagagaggccaaagtccagtggaaagtggacaatgctctgcagagtgggaattcccaggaatctgtcactgagcaggactctaaggatagcacatactccctgtcctctactctgacactgagcaaggctgattacgagaaacacaaagtgtacgcctgtgaagtcacacatcaggggctgtctagtcctgtgaccaaatccttcaataggggagagtgctga

SEQ ID NO: 12 - protein, 214 aa, Artificial Sequence
  Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - DNA, 1368 bp, Artificial Sequence
  Synthetic polynucleotide
gaggtgcagctggtcgagtctggaggaggattggtgcagcctggagggtcattgaaactctcatgtgcagcctctggattcaccttcaatacctacgccatgaactgggtccgccaggctccaggaaagggtttggaatgggttgctcgcataagaagtaaatataataattatgcaacatattatgccgattcagtgaaagacaggttcaccatctccagagatgattcaaaaaacactgcctatctacaaatgaacagcttgaaaactgaggacactgccgtgtactactgtgtgagacatgggaacttcggtgatagctacgtttcctggtttgcttactggggccaagggactctggtcaccgtctcgagcgctagcacaaagggccctagtgtgtttcctctggctccctcttccaaatccacttctggtggcactgctgctctgggatgcctggtgaaggattactttcctgaacctgtgactgtctcatggaactctggtgctctgacttctggtgtccacactttccctgctgtgctgcagtctagtggactgtactctctgtcatctgtggtcactgtgccctcttcatctctgggaacccagacctacatttgtaatgtgaaccacaaaccatccaacactaaagtggacaaaaaagtggaacccaaatcctgtgacaaaacccacacctgcccaccttgtcctgcccctgaactgctgggaggaccttctgtgtttctgttcccccccaaaccaaaggataccctgatgatctctagaacccctgaggtgacatgtgtggtggtggctgtgtctcatgaggaccctgaggtcaaattcaactggtacgtggatggagtggaagtccacaatgccaaaaccaagcctagagaggaacagtacgcttcaacctacagagttgtcagtgtgctgactgtgctgcatcaggattggctgaatggcaaggaatacaagtgtaaagtctcaaacaaggccctggctgctccaattgagaaaacaatctcaaaggccaagggacagcctagggaaccccaggtctacaccctgccaccttcaagagaggaaatgaccaaaaaccaggtgtccctgacatgcctggtcaaaggcttctacccttctgacattgctgtggagtgggagtcaaatggacagcctgagaacaactacaaaacaaccccccctgtgctggattctgatggctctttctttctgtactccaaactgactgtggacaagtctagatggcagcaggggaatgtcttttcttgctctgtcatgcatgaggctctgcataaccactacactcagaaatccctgtctctgtctcccgggaaatga

SEQ ID NO: 14 - protein, 455 aa, Artificial Sequence
  Synthetic polypeptide
EVQLVESGGGLVQPGGSLKLSCAASGFTFNTYAMNWVRQAPGKGLEWVARIRSKYNNYATYYADSVKDRFTISRDDSKNTAYLQMNSLKTEDTAVYYCVRHGNFGDSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - DNA, 648 bp, Artificial Sequence
  Synthetic polynucleotide
cagaccgttgtgactcaggaaccttcactcaccgtatcacctggtggaacagtcacactcacttgtcgctcgtcgactggggctgttacaactagcaactatgccaactgggtccaacaaaaaccgggtcaggcaccccgtggtctaataggtggtaccaacaagcgcgcaccaggtactcctgccagattctcaggctccctgcttggaggcaaggctgccctcaccctctcgggggtacagccagaggatgaggcagaatattactgtgctctatggtacagcaacctctgggtgttcggtggaggaaccaaactgactgtcctaggccagcctaaagcggcgccatccgtcaccctgttccctccctcatccgaggaactgcaggccaataaggctacactggtctgtctgattagcgacttctaccctggggccgtgactgtggcttggaaagccgattcttctcccgtgaaagctggagtggaaacaaccaccccctctaaacagagcaacaacaaatacgctgcctcttcatacctgtccctgacccctgaacagtggaaatctcaccggtcttactcatgccaggtgacacacgagggatcaactgtggagaaaaccgtggctcctaccgaatgttcatga

SEQ ID NO: 16 - protein, 215 aa, Artificial Sequence
  Synthetic polypeptide
QTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 17 - DNA, 1356 bp, Artificial Sequence
  Synthetic polynucleotide
caggtccagctgcagcagagtggggccgaactggcaagacccggagcaagcgtcaaaatgtcatgtaaagcaagcggttatactttcactaggagcaccatgcactgggtgaaacagaggcccggccagggactggagtggatcgggtacattaacccttccagcgcttacaccaactataatcagaagttcaaagacaaggccaccctgacagctgataagtctagttcaacagcatatatgcagctgtccagcctgacttctgaagacagtgcagtgtactattgcgcctccccacaggtccactacgattacaatggttttccttactgggggcagggcacactggtgactgtctccgccgctagcacaaagggccctagtgtgtttcctctggctccctcttccaaatccacttctggtggcactgctgctctgggatgcctggtgaaggattactttcctgaacctgtgactgtctcatggaactctggtgctctgacttctggtgtccacactttccctgctgtgctgcagtctagtggactgtactctctgtcatctgtggtcactgtgccctcttcatctctgggaacccagacctacatttgtaatgtgaaccacaaaccatccaacactaaagtggacaaaaaagtggaacccaaatcctgtgacaaaacccacacctgcccaccttgtcctgcccctgaactgctgggaggaccttctgtgtttctgttcccccccaaaccaaaggataccctgatgatctctagaacccctgaggtgacatgtgtggtggtgaatgtgtctcatgaggaccctgaggtcaaattcaactggtacgtggatggagtggaagtccacaatgccaaaaccaagcctagagaggaacagtacgattcaacctacagagttgtcagtgtgctgactgtgctgcatcaggattggctgaatggcaaggaatacaagtgtaaagtctcaaacaaggccctgggtgctccaattgagaaaacaatctcaaaggccaagggacagcctagggaaccccaggtctacaccctgccaccttcaagagaggaaatgaccaaaaaccaggtgtccctgacatgcctggtcaaaggcttctacccttctgacattgctgtggagtgggagtcaaatggacagcctgagaacaactacaaaacaaccccccctgtgctggattctgatggctctttctttctgtactccaaactgactgtggacaagtctagatggcagcaggggaatgtcttttcttgctctgtcatgcatgaggctctgcataaccactacactcagaaatccctgtctctgtctcccgggaaatga

SEQ ID NO: 18 - protein, 451 aa, Artificial Sequence
  Synthetic polypeptide
QVQLQQSGAELARPGASVKMSCKASGYTFTRSTMHWVKQRPGQGLEWIGYINPSSAYTNYNQKFKDKATLTADKSSSTAYMQLSSLTSEDSAVYYCASPQVHYDYNGFPYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVNVSHEDPEVKFNWYVDGVEVHNAKTKPREEQYDSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - DNA, 1356 bp, Artificial Sequence
  Synthetic polynucleotide
caggtccagctgcagcagagtggggccgaactggcaagacccggagcaagcgtcaaaatgtcatgtaaagcaagcggttatactttcactaggagcaccatgcactgggtgaaacagaggcccggccagggactggagtggatcgggtacattaacccttccagcgcttacaccaactataatcagaagttcaaagacaaggccaccctgacagctgataagtctagttcaacagcatatatgcagctgtccagcctgacttctgaagacagtgcagtgtactattgcgcctccccacaggtccactacgattacaatggttttccttactgggggcagggcacactggtgactgtctccgccgctagcacaaagggccctagtgtgtttcctctggctccctcttccaaatccacttctggtggcactgctgctctgggatgcctggtgaaggattactttcctgaacctgtgactgtctcatggaactctggtgctctgacttctggtgtccacactttccctgctgtgctgcagtctagtggactgtactctctgtcatctgtggtcactgtgccctcttcatctctgggaacccagacctacatttgtaatgtgaaccacaaaccatccaacactaaagtggacaaaaaagtggaacccaaatcctgtgacaaaacccacacctgcccaccttgtcctgcccctgaactgctgggaggaccttctgtgtttctgttcccccccaaaccaaaggataccctgatgatctctagaacccctgaggtgacatgtgtggtggtggaggtgtctcatgaggaccctgaggtcaaattcaactggtacgtggatggagtggaagtccacaatgccaaaaccaagcctagagaggaacagtaccaatcaacctacagagttgtcagtgtgctgactgtgctgcatcaggattggctgaatggcaaggaatacaagtgtaaagtctcaaacaaggccctgtctgctccaattgagaaaacaatctcaaaggccaagggacagcctagggaaccccaggtctacaccctgccaccttcaagagaggaaatgaccaaaaaccaggtgtccctgacatgcctggtcaaaggcttctacccttctgacattgctgtggagtgggagtcaaatggacagcctgagaacaactacaaaacaaccccccctgtgctggattctgatggctctttctttctgtactccaaactgactgtggacaagtctagatggcagcaggggaatgtcttttcttgctctgtcatgcatgaggctctgcataaccactacactcagaaatccctgtctctgtctcccgggaaatga

SEQ ID NO: 20 - protein, 451 aa, Artificial Sequence
  Synthetic polypeptide
QVQLQQSGAELARPGASVKMSCKASGYTFTRSTMHWVKQRPGQGLEWIGYINPSSAYTNYNQKFKDKATLTADKSSSTAYMQLSSLTSEDSAVYYCASPQVHYDYNGFPYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVEVSHEDPEVKFNWYVDGVEVHNAKTKPREEQYQSTYRVVSVLTVLHQDWLNGKEYKCKVSNKALSAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - DNA, 885 bp, Artificial Sequence
  Synthetic polynucleotide
cagaccgttgtgactcaggaaccttcactcaccgtatcacctggtggaacagtcacactcacttgtcgctcgtcgactggggctgttacaactagcaactatgccaactgggtccaacaaaaaccgggtcaggcaccccgtggtctaataggtggtaccaacaagcgcgcaccaggtactcctgccagattctcaggctccctgcttggaggcaaggctgccctcaccctctcgggggtacagccagaggatgaggcagaatattactgtgctctatggtacagcaacctctgggtgttcggtggaggaaccaaactgactgtcctaggccagcctaaagcggcgccatccgtcaccctgttccctccctcatccgaggaactgcaggccaataaggctacactggtctgtctgattagcgacttctaccctggggccgtgactgtggcttggaaagccgattcttctcccgtgaaagctggagtggaaacaaccaccccctctaaacagagcaacaacaaatacgctgcctcttcatacctgtccctgacccctgaacagtggaaatctcaccggtcttactcatgccaggtgacacacgagggatcaactgtggagaaaaccgtggctcctaccgaatgttcaggcggtggaggatccgggggtgggggaagcggcggaggaggtagcggcgtgactctgttcgtcgctctgtacgactatgaggccctgggggctcacgaactgtccttccataagggcgagaaatttcagatcctgtcccccaggagcgagggacctttttgggaagcacactctctgaccacaggcgaaaccggatggattccctctaactacgtggcccccgtcgatagtattcagtga

SEQ ID NO: 22 - protein, 294 aa, Artificial Sequence
  Synthetic polypeptide
QTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSGGGGSGGGGSGGGGSGVTLFVALYDYEALGAHELSFHKGEKFQILSPRSEGPFWEAHSLTTGETGWIPSNYVAPVDSIQ

SEQ ID NO: 23 - DNA, 885 bp, Artificial Sequence
  Synthetic polynucleotide
cagaccgttgtgactcaggaaccttcactcaccgtatcacctggtggaacagtcacactcacttgtcgctcgtcgactggggctgttacaactagcaactatgccaactgggtccaacaaaaaccgggtcaggcaccccgtggtctaataggtggtaccaacaagcgcgcaccaggtactcctgccagattctcaggctccctgcttggaggcaaggctgccctcaccctctcgggggtacagccagaggatgaggcagaatattactgtgctctatggtacagcaacctctgggtgttcggtggaggaaccaaactgactgtcctaggccagcctaaagcggcgccatccgtcaccctgttccctccctcatccgaggaactgcaggccaataaggctacactggtctgtctgattagcgacttctaccctggggccgtgactgtggcttggaaagccgattcttctcccgtgaaagctggagtggaaacaaccaccccctctaaacagagcaacaacaaatacgctgcctcttcatacctgtccctgacccctgaacagtggaaatctcaccggtcttactcatgccaggtgacacacgagggatcaactgtggagaaaaccgtggctcctaccgaatgttcaggcggtggaggatccgggggtgggggaagcggcggaggaggtagcggcgtgactctgttcgtcgctctgtacgactatgaggccctgggggctcacgaactgtccttccataagggcgagaaatttcagatcctgtccagcctggcagtgggaccattttgggaggcccactctctgaccacaggcgaaaccggatggattccctctaactacgtggcacctgtcgatagtattcagtga

SEQ ID NO: 24 - protein, 294 aa, Artificial Sequence
  Synthetic polypeptide
QTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSGGGGSGGGGSGGGGSGVTLFVALYDYEALGAHELSFHKGEKFQILSSLAVGPFWEAHSLTTGETGWIPSNYVAPVDSIQ

SEQ ID NO: 25 - DNA, 1350 bp, Artificial Sequence
  Synthetic polynucleotide
caggtgcagctggtgcagtctggcggcggagtggtgcagcctggaagatccctgcggctgtcctgcaaggcctccggctacaccttcacccggtacaccatgcactgggtgcgacaggcccctggcaagggcctggaatggatcggctacatcaacccctcccggggctacaccaactacaaccagaaagtgaaggaccggttcaccatctcccgggacaactccaagaacaccgcctttctgcagatggacagcctgcggcctgaggataccggcgtgtacttctgcgcccggtactacgacgaccactactgcctggactactggggccagggcacccctgtgacagtgtcctctgctagcacaaagggccctagtgtgtttcctctggctccctcttccaaatccacttctggtggcactgctgctctgggatgcctggtgaaggattactttcctgaacctgtgactgtctcatggaactctggtgctctgacttctggtgtccacactttccctgctgtgctgcagtctagtggactgtactctctgtcatctgtggtcactgtgccctcttcatctctgggaacccagacctacatttgtaatgtgaaccacaaaccatccaacactaaagtggacaaaaaagtggaacccaaatcctgtgacaaaacccacacctgcccaccttgtcctgcccctgaagccgccggaggaccttctgtgtttctgttcccccccaaaccaaaggataccctgatgatctctagaacccctgaggtgacatgtgtggtggtggatgtgtctcatgaggaccctgaggtcaaattcaactggtacgtggatggagtggaagtccacaatgccaaaaccaagcctagagaggaacagtacaattcaacctacagagtggtcagtgtgctgactgtgctgcatcaggattggctgaatggcaaggaatacaagtgtaaagtctcaaacaaggccctgcctgctccaattgagaaaacaatctcaaaggccaagggacagcctagggaaccccaggtctacaccctgccaccttcaagagaggaaatgaccaaaaaccaggtgtccctgacatgcctggtcaaaggcttctacccttctgacattgctgtggagtgggagtcaaatggacagcctgagaacaactacaaaacaaccccccctgtgctggattctgatggctctttctttctgtactccaaactgactgtggacaagtctagatggcagcaggggaatgtcttttcttgctctgtcatgcatgaggctctgcataaccactacactcagaaatccctgtctctgtctcccgggaaatga

SEQ ID NO: 26 - protein, 449 aa, Artificial Sequence
  Synthetic polypeptide
QVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - DNA, 876 bp, Artificial Sequence
  Synthetic polynucleotide
gacatccagatgacccagtccccctccagcctgtctgcctctgtgggcgacagagtgacaattacctgctccgcctcctcctccgtgtcctacatgaactggtatcagcagacccccggcaaggcccccaagcggtggatctacgacacctccaagctggcctctggcgtgccctccagattctccggctctggctctggcaccgactataccttcaccatcagctccctgcagcccgaggatatcgccacctactactgccagcagtggtcctccaaccccttcacctttggccagggcaccaagctgcagatcacccgtacggtcgcggcgccttctgtgttcattttccccccatctgatgaacagctgaaatctggcactgcttctgtggtctgtctgctgaacaacttctaccctagagaggccaaagtccagtggaaagtggacaatgctctgcagagtgggaattcccaggaatctgtcactgagcaggactctaaggatagcacatactccctgtcctctactctgacactgagcaaggctgattacgagaaacacaaagtgtacgcctgtgaagtcacacatcaggggctgtctagtcctgtgaccaaatccttcaataggggagagtgcggcggtggaggatccgggggtgggggaagcggcggaggaggtagcggcgtgaccctgtttgtggccctgtacgactacgaggccctgggcgctcacgagctgtctttccacaagggcgagaagttccagatcctgaactcctccgagggccccttctgggaggctcactctctgacaaccggcgagacaggctggattccctccaactatgtggcccccgtggactccatccagtga

SEQ ID NO: 28 - protein, 291 aa, Artificial Sequence
  Synthetic polypeptide
DIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGVTLFVALYDYEALGAHELSFHKGEKFQILNSSEGPFWEAHSLTTGETGWIPSNYVAPVDSIQ

SEQ ID NO: 29 - DNA, 1353 bp, Artificial Sequence
  Synthetic polynucleotide
cagatcaccctgaaggagtccgggcccacactggtgaaacctactcagaccctgacactgacttgcaccttctccggtttttctctgagtacctcgggcatgggagtgagctggatcaggcagccccctggcaaggcactggaatggctggcccacatctactgggacgatgacaagaggtacaacccttcactgaaatcccggctgacaattactaaggataccagcaaaaaccaggtggtcctgaccatgacaaatatggaccccgtggacactgctacctactattgtgcaagactgtacggcttcacctatggatttgcttactgggggcagggcaccctggtcacagtctcgagcgctagcacaaagggccctagtgtgtttcctctggctccctcttccaaatccacttctggtggcactgctgctctgggatgcctggtgaaggattactttcctgaacctgtgactgtctcatggaactctggtgctctgacttctggtgtccacactttccctgctgtgctgcagtctagtggactgtactctctgtcatctgtggtcactgtgccctcttcatctctgggaacccagacctacatttgtaatgtgaaccacaaaccatccaacactaaagtggacaaaaaagtggaacccaaatcctgtgacaaaacccacacctgcccaccttgtcctgcccctgaactgctgggaggaccttctgtgtttctgttcccccccaaaccaaaggataccctgatgatctctagaacccctgaggtgacatgtgtggtggtggatgtgtctcatgaggaccctgaggtcaaattcaactggtacgtggatggagtggaagtccacaatgccaaaaccaagcctagagaggaacagtacaattcaacctacagagttgtcagtgtgctgactgtgctgcatcaggattggctgaatggcaaggaatacaagtgtaaagtctcaaacaaggccctgcctgctccaattgagaaaacaatctcaaaggccaagggacagcctagggaaccccaggtctacaccctgccaccttcaagagaggaaatgaccaaaaaccaggtgtccctgacatgcctggtcaaaggcttctacccttctgacattgctgtggagtgggagtcaaatggacagcctgagaacaactacaaaacaaccccccctgtgctggattctgatggctctttctttctgtactccaaactgactgtggacaagtctagatggcagcaggggaatgtcttttcttgctctgtcatgcatgaggctctgcataaccactacactcagaaatccctgtctctgtctcccgggaaatga

SEQ ID NO: 30 - protein, 450 aa, Artificial Sequence
  Synthetic polypeptide
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - DNA, 657 bp, Artificial Sequence
  Synthetic polynucleotide
gacatcgtgatgacacagagcccagattctctggccgtcagcctgggcgaaagggccactatcaactgccgggcctcccagtctgtggactacaatggaatttcttacatgcactggtatcagcagaagcctggccagccccctaaactgctgatctatgccgcttcaaaccctgagtccggcgtgccagaccgattcagtggctcaggctccgggaccgattttaccctgacaatttccagcctgcaagctgaggacgtggcagtctactattgccagcagatcattgaagatccctggacattcggtcagggcactaaggtggagatcaaacgtacggtcgcggcgccttctgtgttcattttccccccatctgatgaacagctgaaatctggcactgcttctgtggtctgtctgctgaacaacttctaccctagagaggccaaagtccagtggaaagtggacaatgctctgcagagtgggaattcccaggaatctgtcactgagcaggactctaaggatagcacatactccctgtcctctactctgacactgagcaaggctgattacgagaaacacaaagtgtacgcctgtgaagtcacacatcaggggctgtctagtcctgtgaccaaatccttcaataggggagagtgctga

SEQ ID NO: 32 - protein, 218 aa, Artificial Sequence
  Synthetic polypeptide
DIVMTQSPDSLAVSLGERATINCRASQSVDYNGISYMHWYQQKPGQPPKLLIYAASNPESGVPDRFSGSGSGTDFTLTISSLQAEDVAVYYCQQIIEDPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - DNA, 1353 bp, Artificial Sequence
  Synthetic polynucleotide
cagatcaccctgaaggagtccgggcccacactggtgaaacctactcagaccctgacactgacttgcaccttctccggtttttctctgagtacctcgggcatgggagtgagctggatcaggcagccccctggcaaggcactggaatggctggcccacatctactgggacgatgacaagaggtacaacccttcactgaaatcccggctgacaattactaaggataccagcaaaaaccaggtggtcctgaccatgacaaatatggaccccgtggacactgctacctactattgtgcaagactgtacggcttcacctatggatttgcttactgggggcagggcaccctggtcacagtctcgagcgctagcacaaagggccctagtgtgtttcctctggctccctcttccaaatccacttctggtggcactgctgctctgggatgcctggtgaaggattactttcctgaacctgtgactgtctcatggaactctggtgctctgacttctggtgtccacactttccctgctgtgctgcagtctagtggactgtactctctgtcatctgtggtcactgtgccctcttcatctctgggaacccagacctacatttgtaatgtgaaccacaaaccatccaacactaaagtggacaaaaaagtggaacccaaatcctgtgacaaaacccacacctgcccaccttgtcctgcccctgaagccgccggaggaccttctgtgtttctgttcccccccaaaccaaaggataccctgatgatctctagaacccctgaggtgacatgtgtggtggtggatgtgtctcatgaggaccctgaggtcaaattcaactggtacgtggatggagtggaagtccacaatgccaaaaccaagcctagagaggaacagtacaattcaacctacagagtggtcagtgtgctgactgtgctgcatcaggattggctgaatggcaaggaatacaagtgtaaagtctcaaacaaggccctgcctgctccaattgagaaaacaatctcaaaggccaagggacagcctagggaaccccaggtctacaccctgccaccttcaagagaggaaatgaccaaaaaccaggtgtccctgacatgcctggtcaaaggcttctacccttctgacattgctgtggagtgggagtcaaatggacagcctgagaacaactacaaaacaaccccccctgtgctggattctgatggctctttctttctgtactccaaactgactgtggacaagtctagatggcagcaggggaatgtcttttcttgctctgtcatgcatgaggctctgcataaccactacactcagaaatccctgtctctgtctcccgggaaatga

SEQ ID NO: 34 - protein, 450 aa, Artificial Sequence
  Synthetic polypeptide
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - DNA, 195 bp, Artificial Sequence
  Synthetic polynucleotide
ggcgtgactctgttcgtcgctctgtacgactatgaggccctgggggctcacgaactgtccttccataagggcgagaaatttcagatcctgtcccccaggagcgagggacctttttgggaagcacactctctgaccacaggcgaaaccggatggattccctctaactacgtggcccccgtcgatagtattcagtga

SEQ ID NO: 36 - protein, 64 aa, Artificial Sequence
  Synthetic polypeptide
GVTLFVALYDYEALGAHELSFHKGEKFQILSPRSEGPFWEAHSLTTGETGWIPSNYVAPVDSIQ

SEQ ID NO: 37 - DNA, 195 bp, Artificial Sequence
  Synthetic polynucleotide
ggcgtgactctgttcgtcgctctgtacgactatgaggccctgggggctcacgaactgtccttccataagggcgagaaatttcagatcctgtccagcctggcagtgggaccattttgggaggcccactctctgaccacaggcgaaaccggatggattccctctaactacgtggcacctgtcgatagtattcagtga

SEQ ID NO: 38 - protein, 64 aa, Artificial Sequence
  Synthetic polypeptide
GVTLFVALYDYEALGAHELSFHKGEKFQILSSLAVGPFWEAHSLTTGETGWIPSNYVAPVDSIQ

SEQ ID NO: 39 - DNA, 45 bp, Artificial Sequence
  Synthetic oligonucleotide
ggcggtggaggatccgggggtgggggaagcggcggaggaggtagc

SEQ ID NO: 40 - protein, 15 aa, Artificial Sequence
  Synthetic peptide
GGGGSGGGGSGGGGS

SEQ ID NO: 41 - DNA, 57 bp, Artificial Sequence
  Synthetic oligonucleotide
atgaattttggactgaggctgattttcctggtgctgaccctgaaaggcgtccagtgt

SEQ ID NO: 42 - protein, 19 aa, Artificial Sequence
  Synthetic peptide
MNFGLRLIFLVLTLKGVQC

SEQ ID NO: 43 - protein, 222 aa, Homo sapiens
CPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - DNA, 1356 bp, Artificial Sequence
  Synthetic polynucleotide
caggtccagctgcagcagagtggggccgaactggcaagacccggagcaagcgtcaaaatgtcatgtaaagcaagcggttatactttcactaggagcaccatgcactgggtgaaacagaggcccggccagggactggagtggatcgggtacattaacccttccagcgcttacaccaactataatcagaagttcaaagacaaggccaccctgacagctgataagtctagttcaacagcatatatgcagctgtccagcctgacttctgaagacagtgcagtgtactattgcgcctccccacaggtccactacgattacaatggttttccttactgggggcagggcacactggtgactgtctccgccgctagcacaaagggccctagtgtgtttcctctggctccctcttccaaatccacttctggtggcactgctgctctgggatgcctggtgaaggattactttcctgaacctgtgactgtctcatggaactctggtgctctgacttctggtgtccacactttccctgctgtgctgcagtctagtggactgtactctctgtcatctgtggtcactgtgccctcttcatctctgggaacccagacctacatttgtaatgtgaaccacaaaccatccaacactaaagtggacaaaaaagtggaacccaaatcctgtgacaaaacccacacctgcccaccttgtcctgcccctgaactgctgggaggaccttctgtgtttctgttcccccccaaaccaaaggataccctgatgatctctagaacccctgaggtgacatgtgtggtggtggctgtgtctcatgaggaccctgaggtcaaattcaactggtacgtggatggagtggaagtccacaatgccaaaaccaagcctagagaggaacagtacgcttcaacctacagagttgtcagtgtgctgactgtgctgcatcaggattggctgaatggcaaggaatacaagtgtaaagtctcaaacaaggccctggctgctccaattgagaaaacaatctcaaaggccaagggacagcctagggaaccccaggtctacaccctgccaccttcaagagaggaaatgaccaaaaaccaggtgtccctgacatgcctggtcaaaggcttctacccttctgacattgctgtggagtgggagtcaaatggacagcctgagaacaactacaaaacaaccccccctgtgctggattctgatggctctttctttctgtactccaaactgactgtggacaagtctagatggcagcaggggaatgtcttttcttgctctgtcatgcatgaggctctgcataaccactacactcagaaatccctgtctctgtctcccgggaaatga

SEQ ID NO: 45 - protein, 451 aa, Artificial Sequence
  Synthetic polypeptide“
QVQLQQSGAELARPGASVKMSCKASGYTFTRSTMHWVKQRPGQGLEWIGYINPSSAYTNYNQKFKDKATLTADKSSSTAYMQLSSLTSEDSAVYYCASPQVHYDYNGFPYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - DNA, 1356 bp, Artificial Sequence
  Synthetic polynucleotide
caggtccagctgcagcagagtggggccgaactggcaagacccggagcaagcgtcaaaatgtcatgtaaagcaagcggttatactttcactaggagcaccatgcactgggtgaaacagaggcccggccagggactggagtggatcgggtacattaacccttccagcgcttacaccaactataatcagaagttcaaagacaaggccaccctgacagctgataagtctagttcaacagcatatatgcagctgtccagcctgacttctgaagacagtgcagtgtactattgcgcctccccacaggtccactacgattacaatggttttccttactgggggcagggcacactggtgactgtctccgccgctagcacaaagggccctagtgtgtttcctctggctccctcttccaaatccacttctggtggcactgctgctctgggatgcctggtgaaggattactttcctgaacctgtgactgtctcatggaactctggtgctctgacttctggtgtccacactttccctgctgtgctgcagtctagtggactgtactctctgtcatctgtggtcactgtgccctcttcatctctgggaacccagacctacatttgtaatgtgaaccacaaaccatccaacactaaagtggacaaaaaagtggaacccaaatcctgtgacaaaacccacacctgcccaccttgtcctgcccctgaactgctgggaggaccttctgtgtttctgttcccccccaaaccaaaggataccctgatgatctctagaacccctgaggtgacatgtgtggtggtggctgtgtctcatgaggaccctgaggtcaaattcaactggtacgtggatggagtggaagtccacaatgccaaaaccaagcctagagaggaacagtacaattcaacctacagagttgtcagtgtgctgactgtgctgcatcaggattggctgaatggcaaggaatacaagtgtaaagtctcaaacaaggccctggctgctccaattgagaaaacaatctcaaaggccaagggacagcctagggaaccccaggtctacaccctgccaccttcaagagaggaaatgaccaaaaaccaggtgtccctgacatgcctggtcaaaggcttctacccttctgacattgctgtggagtgggagtcaaatggacagcctgagaacaactacaaaacaaccccccctgtgctggattctgatggctctttctttctgtactccaaactgactgtggacaagtctagatggcagcaggggaatgtcttttcttgctctgtcatgcatgaggctctgcataaccactacactcagaaatccctgtctctgtctcccgggaaatga

SEQ ID NO: 47 - protein, 451 aa, Artificial Sequence
  Synthetic polypeptide
QVQLQQSGAELARPGASVKMSCKASGYTFTRSTMHWVKQRPGQGLEWIGYINPSSAYTNYNQKFKDKATLTADKSSSTAYMQLSSLTSEDSAVYYCASPQVHYDYNGFPYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - DNA, 1356 bp, Artificial Sequence
  Synthetic polynucleotide
caggtccagctgcagcagagtggggccgaactggcaagacccggagcaagcgtcaaaatgtcatgtaaagcaagcggttatactttcactaggagcaccatgcactgggtgaaacagaggcccggccagggactggagtggatcgggtacattaacccttccagcgcttacaccaactataatcagaagttcaaagacaaggccaccctgacagctgataagtctagttcaacagcatatatgcagctgtccagcctgacttctgaagacagtgcagtgtactattgcgcctccccacaggtccactacgattacaatggttttccttactgggggcagggcacactggtgactgtctccgccgctagcacaaagggccctagtgtgtttcctctggctccctcttccaaatccacttctggtggcactgctgctctgggatgcctggtgaaggattactttcctgaacctgtgactgtctcatggaactctggtgctctgacttctggtgtccacactttccctgctgtgctgcagtctagtggactgtactctctgtcatctgtggtcactgtgccctcttcatctctgggaacccagacctacatttgtaatgtgaaccacaaaccatccaacactaaagtggacaaaaaagtggaacccaaatcctgtgacaaaacccacacctgcccaccttgtcctgcccctgaactgctgggaggaccttctgtgtttctgttcccccccaaaccaaaggataccctgatgatctctagaacccctgaggtgacatgtgtggtggtggatgtgtctcatgaggaccctgaggtcaaattcaactggtacgtggatggagtggaagtccacaatgccaaaaccaagcctagagaggaacagtacgcttcaacctacagagttgtcagtgtgctgactgtgctgcatcaggattggctgaatggcaaggaatacaagtgtaaagtctcaaacaaggccctgcctgctccaattgagaaaacaatctcaaaggccaagggacagcctagggaaccccaggtctacaccctgccaccttcaagagaggaaatgaccaaaaaccaggtgtccctgacatgcctggtcaaaggcttctacccttctgacattgctgtggagtgggagtcaaatggacagcctgagaacaactacaaaacaaccccccctgtgctggattctgatggctctttctttctgtactccaaactgactgtggacaagtctagatggcagcaggggaatgtcttttcttgctctgtcatgcatgaggctctgcataaccactacactcagaaatccctgtctctgtctcccgggaaatga

SEQ ID NO: 49 - protein, 451 aa, Artificial Sequence
  Synthetic polypeptide
QVQLQQSGAELARPGASVKMSCKASGYTFTRSTMHWVKQRPGQGLEWIGYINPSSAYTNYNQKFKDKATLTADKSSSTAYMQLSSLTSEDSAVYYCASPQVHYDYNGFPYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - DNA, 1356 bp, Artificial Sequence
  Synthetic polynucleotide
caggtccagctgcagcagagtggggccgaactggcaagacccggagcaagcgtcaaaatgtcatgtaaagcaagcggttatactttcactaggagcaccatgcactgggtgaaacagaggcccggccagggactggagtggatcgggtacattaacccttccagcgcttacaccaactataatcagaagttcaaagacaaggccaccctgacagctgataagtctagttcaacagcatatatgcagctgtccagcctgacttctgaagacagtgcagtgtactattgcgcctccccacaggtccactacgattacaatggttttccttactgggggcagggcacactggtgactgtctccgccgctagcacaaagggccctagtgtgtttcctctggctccctcttccaaatccacttctggtggcactgctgctctgggatgcctggtgaaggattactttcctgaacctgtgactgtctcatggaactctggtgctctgacttctggtgtccacactttccctgctgtgctgcagtctagtggactgtactctctgtcatctgtggtcactgtgccctcttcatctctgggaacccagacctacatttgtaatgtgaaccacaaaccatccaacactaaagtggacaaaaaagtggaacccaaatcctgtgacaaaacccacacctgcccaccttgtcctgcccctgaagccgccggaggaccttctgtgtttctgttcccccccaaaccaaaggataccctgatgatctctagaacccctgaggtgacatgtgtggtggtggatgtgtctcatgaggaccctgaggtcaaattcaactggtacgtggatggagtggaagtccacaatgccaaaaccaagcctagagaggaacagtacaattcaacctacagagtggtcagtgtgctgactgtgctgcatcaggattggctgaatggcaaggaatacaagtgtaaagtctcaaacaaggccctgcctgctccaattgagaaaacaatctcaaaggccaagggacagcctagggaaccccaggtctacaccctgccaccttcaagagaggaaatgaccaaaaaccaggtgtccctgacatgcctggtcaaaggcttctacccttctgacattgctgtggagtgggagtcaaatggacagcctgagaacaactacaaaacaaccccccctgtgctggattctgatggctctttctttctgtactccaaactgactgtggacaagtctagatggcagcaggggaatgtcttttcttgctctgtcatgcatgaggctctgcataaccactacactcagaaatccctgtctctgtctcccgggaaatga

SEQ ID NO: 51 - protein, 451 aa, Artificial Sequence
  Synthetic polypeptide
QVQLQQSGAELARPGASVKMSCKASGYTFTRSTMHWVKQRPGQGLEWIGYINPSSAYTNYNQKFKDKATLTADKSSSTAYMQLSSLTSEDSAVYYCASPQVHYDYNGFPYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 222 aa, Homo sapiens
CPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 222 aa, Homo sapiens
CPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 222 aa, Homo sapiens
CPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEGLHNHYTQKSLSLSPGK

SEQ ID NO: 55 - protein, 222 aa, Homo sapiens
CPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 222 aa, Homo sapiens
CPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEMTKNQVSLTCLVKGFYPSDIAVEWESNGQPENNYKTTPPVLDSDGSFFLYSKLTVDKSRWQQGNVFSCSVMHEGLHNHYTQKSLSLSPGK

SEQ ID NO: 57 - protein, 222 aa, Homo sapiens
CPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREELTKNQVSLTCLVKGFYPSDIAVEWESNGQPENNYKTTPPVLDSDGSFFLYSKLTVDKSRWQQGNVFSCSVMHEGLHNHYTQKSLSLSPGK

SEQ ID NO: 58 - protein, 222 aa, Homo sapiens
CPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEGLHNHYTQKSLSLSPGK

SEQ ID NO: 59 - protein, 63 aa, Artificial Sequence
  Synthetic polypeptide
GVTLFVALYDYEARTEDDLSFHKGEKFQILNSSEGDWWEARSLTTGETGYIPSNYVAPVDSIQ

SEQ ID NO: 60 - protein, 110 aa, human
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 61 - protein, 107 aa, human
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2019/0100587 A1
(43) Pub. Date: Apr. 4, 2019

(54) IgG1 Fc MUTANTS WITH ABLATED EFFECTOR FUNCTIONS

(71) Applicant: Covagen AG, Zug (CH)

(72) Inventors: Simon Brack, Winterthur (CH); Isabella Attinger-Toller, Zurich (CH); Fabian Buller, Unterengstringen (CH); Dragan Grabulovski, Zurich (CH); Julian Bertschinger, Zurich (CH); Adrian Zumsteg, Zurich (CH)

(73) Assignee: Covagen AG, Zug (CH)

(21) Appl. No.: 16/139,500

(22) Filed: Sep. 24, 2018

(30) Foreign Application Priority Data
Oct. 2, 2017 (EP) 17194368.1
Apr. 24, 2018 (EP) 18168959.7

(51) Int. Cl. C07K 16/28

(52) U.S. Cl. CPC C07K 16/2809; C07K 16/2803; C07K 2317/52; C07K 2317/524; C07K 2317/21; C07K 2319/30; C07K 2317/71; C07K 2317/92; C07K 2317/732; C07K 2317/734; C07K 2317/76; C07K 2317/31; C07K 2317/14

(57) ABSTRACT

Antibody and other Fc-containing molecules with variations in the Fc region with reduced binding to C1q and Fc gamma receptors are provided, which can be used in the treatment of various diseases and disorders.

### TECHNICAL FIELD

The disclosure provided herein relates to human antibody IgG1 constant regions (Fc regions) mutated such that they retain FcRn binding, but substantially lose the capacity to specifically bind Fcγ receptors and C1q.

### BACKGROUND

Immunoglobulins, which are glycoproteins present in the serum, tissue, or body fluid of every mammal, have the function of recognizing foreign antigens. The immunoglobulins participate through antibody binding to antigens in biophylaxis via the activation of the complement system or via the activation of effector functions such as enhancement in cellular phagocytosis, antibody-dependent cytotoxicity, and mediator release triggered by interactions with an Fc receptor (FcR) present on the effector cell surface.

Human immunoglobulins are divided into 5 different classes consisting of IgG, IgA, IgM, IgD, and IgE. IgG can further be classified into 4 subclasses consisting of IgG1, IgG2, IgG3, and IgG4, while IgA can further be classified into two subclasses consisting of IgA1 and IgA2. The basic structure of immunoglobulin comprises two homologous light chains (L chains) and 2 homologous heavy chains (H chains). The immunoglobulin classes and subclasses are determined depending on H chains.

Different types of immunoglobulins are known to have different functions. For example, complement-binding ability is high in IgM>IgG3>IgG1>IgG2 in this order, and affinity for Fcγ receptor I is high in IgG3>IgG1>IgG4>IgG2 in this order. Moreover, IgG1, IgG2, and IgG4 are capable of binding to Protein A.

Many monoclonal antibodies have undergone clinical trials in recent years and have been placed on the market for pharmaceutical applications. The majority of these monoclonal antibodies are derived from the IgG1 subclass.

Using IgG1 as the starting point, efforts have been made to generate Fc-containing molecules with diminished Fc receptor binding and effector functions but which retain FcRn binding, prolonged stability, and low immunogenicity. These types of molecules may provide improved antibody therapeutics, for example with a better safety profile.

IgG antibodies are bifunctional molecules in the sense that besides antigen-specific binding via their Fab arms, they are capable of engaging via their Fcγ domain with Fcγ receptors (FcγR) (Woof J M and D R Burton (2004). Nat Rev Immunol 4(2): 89-99). There are three types receptors for Fcγ, FcγRI-FcγRIII, with different affinities for IgG (Bruhns P, et al. (2009). Blood 113(16): 3716-3725). FcγR are expressed on various cell types which mediate Fcγ-mediated immune effector functions such as antibody-dependent cell-mediated cytotoxicity (ADCC) or antibody dependent cellular phagocytosis (ADCP). The antibody Fcγ domain also binds to the complement factor C1q and thereby can activate the complement pathway, ultimately leading to complement-dependent cytotoxicity (CDC). Effector functions mediated by FcγR or C1q are believed to play a role in the activity of several therapeutic antibodies (Redman J M, et al. (2015). Mol Immunol 67(2 Pt A): 28-45.

In addition to inducing effects via FcγR expressing immune cells, engagement of FcγR forms higher order clusters of antibody which causes higher order clustering, or cross-linking, of cell-membrane antigens bound by the antibody Fab, thereby triggering downstream signaling (Stewart R H, et al. (2014). Journal of ImmunoTherapy of Cancer 2(29)). As an example, CD40-specific antibodies have been shown to activate CD40 downstream signaling in an FcγR dependent fashion (White A L, et al. (2011). J Immunol 187(4): 1754-1763).

While for many therapeutic antibody applications, it is desirable to have strong Fcγ mediated effector functions, certain applications rely on mode of actions that do not require effector functions or even necessitate inert antibodies that do not induce FcγR mediated effects. For this reason, IgG isotypes with reduced effector functions (e.g. IgG2 or IgG4) or engineered Fcγ sequences with mutations in the Fcγ-FcγR interface that reduce the affinity to FcγR are utilized in such antibodies. For example, anti-tumor antibodies that boost T cells by blocking T cell inhibitory receptors such as PD-1 or PD-L1 are preferentially inert, since a fully competent Fc region counteracts the antibody's mode of action by depletion of T cells via ADCC or CDC (Stewart R H, et al. (2014). Journal of ImmunoTherapy of Cancer 2(29)). Thus, there is a wide application for antibodies with reduced or abrogated binding to FcγR.

The field has found several solutions to the technical challenge of finding Fcγ with reduced affinity to FcγR by introduction of targeted mutations in the antibody Fcγ domain. Nose and Wigzell described that antibodies without N-linked carbohydrate at N297 did not bind to FcγR expressing cells and lacked ADCC activity (Nose M and H Wigzell (1983). Proc Natl Acad Sci USA 80(21): 6632-6636). Tao and Morrison, and Bolt et al. performed site-directed mutagenesis at position N297, resulting in aglycosylated antibodies with reduced binding to FcγR and C1q (Tao M H and S L Morrison (1989). J Immunol 143(8): 2595-2601; Bolt S, et al. (1993). Eur J Immunol 23(2): 403-411). Several clinical-stage antibodies or Fcγ-fusion proteins carry mutations at N297, for instance anti-PDL 1 mAb atezolizumab, anti-GITR mAb TRX518, anti-CD3 mAb otelixizumab or the peptide-Fcγ fusion protein romiplostim (Strohl W R (2009). Curr Opin Biotechnol 20(6): 685-691; Stewart R H, et al. (2014). Journal of ImmunoTherapy of Cancer 2(29)).

CD3 specific antibodies induce FcR-dependent T cell activation and cytokine release (Parren P W, et al. (1992). J Immunol 148(3): 695-701; Xu D, et al. (2000). Cell Immunol 200(1): 16-26). It was observed that a CD3-specific IgG1 antibody with a N297A mutation in the Fcγ still leads to T cell activation (WO2012143524), despite the fact the N297A has been described to have no detectable binding to FcγR expressing cells (Bolt S, et al. (1993). Eur J Immunol 23(2): 403-411). These observations suggest that in vitro T cell activation assays with CD3-specific antibodies are very sensitive to residual FcγR binding. Thus, in vitro T cell assays with CD3 antibodies represent an optimal functional assay to identify engineered Fcγ sequences with no or minimal binding affinity to FcγR.

Canfield and Morrison described that the hinge region of IgG contributes to binding to the high-affinity FcγRI (Canfield S M and S L Morrison (1991). J Exp Med 173(6): 1483-1491). Xu et al. demonstrated that the humanized anti-CD3 antibody hOKT3 containing the double mutations L234A and L235A in the lower hinge (also termed “Ala-Ala”, or “LALA”) demonstrated reduced C1q and FcγR binding, leading to dampened FcγR-mediated T cell activation and cytokine release in vitro (Xu D, et al. (2000). Cell Immunol 200(1): 16-26). This antibody, termed hOKT3γ1(Ala-Ala) or teplizumab, was subsequently investigated in clinical trials, where it was found that the introduction of the LALA mutations led to a reduced incidence of adverse cytokine release (Herold K C, et al. (2005). Diabetes 54(6): 1763-1769).

(Shields et al. (2001), J Biol Chem 276(9): 6591-6604) performed an alanine scanning mutagenesis approach on the entire antibody Fcγ to identify residues that contribute to FcγR binding. They found that mutation of D265 decreased the affinity to all Fcγ receptors. In addition, mutating position P329 was shown to reduce binding to Fcγ receptors. Idusogie et al. mapped the C1q binding site of rituximab, a chimeric IgG1 antibody, and found that mutations at D270, K322, P329 or P331 reduced binding to C1q (Idusogie E E, et al. (2000). J Immunol 164(8): 4178-4184). Wilson et al. described a combination of mutations at D265 and N297 to alanine, termed “DANA”. These combined mutations were claimed to have reduced binding to FcγR, but residual binding to mouse FcγRIII was detected (Wilson N S, et al. (2011). Cancer Cell 19(1): 101-113). Gong et al. described that the “DANA” mutations exhibit partial reduction in complement activation (Gong Q, et al. (2005). J Immunol 174(2): 817-826).

Other reports described mutations of certain amino acids in the Fc part of antibodies (e.g. D265N and D265E: Shields R L, et al. (2001) J Biol Chem 276: 6591-6604; N297Q: Stavenhagen J B, et al. (2007) Cancer Res 67: 8882-8890; N297D: Sazinsky S L, et al. (2008) Proc Natl Acad Sci USA 105: 20167-20172, Kelton W, et al. (2014) Chem Biol 21: 1603-1609; P329G: Schlothauer T, et al. (2016) Protein Eng Des Sel 29: 457-466), although not all these studies related to impairment of Fc functionality.

Several Fcγ mutations were described that reduce binding to FcγR, but none of those mentioned above completely abrogate FcγR binding and C1q binding. Shields et al. have described the possibility to further reduce FcγR binding by combining individual mutations (Shields R L, et al. (2001). J Biol Chem 276(9): 6591-6604), a concept that is also underlying the “LALA” or “DANA” combination mutations described above.

However, the challenge remains to identify a combination of mutations that results in optimally reduced FcγR binding and importantly, that does not negatively impact other key properties that are of importance to a pharmaceutical product, such as manufacturability, pharmacokinetics, or antigenicity.

For example, WO 2014/108483 describes several Fcγ sequences containing combinations of mutations with reduced FcγR binding. The majority of the Fcγ-mutated antibodies had a faster clearance in mice than the corresponding unmodified IgG1 antibody. Therefore, introducing mutations in Fcγ domains is known to potentially have an impact on the pharmacokinetic properties.

The Fcγ domain also interacts with the neonatal Fc receptor, FcRn (Kuo T T and V G Aveson (2011). MAbs 3(5): 422-430). This interaction is responsible for antibody recycling, rescue from lysosomal degradation and thus for the long half-life of IgG1 antibodies. The FcRn binding site is located in the CH2-CH3 interface of Fcγ1 (Martin W L, et al. (2001). Mol Cell 7(4): 867-877). Therefore, novel engineered Fcγ domains with mutations in the CH2 domain can have impaired FcRn affinity and therefore impaired pharmacokinetic properties (Shields R L, et al. (2001). J Biol Chem 276(9): 6591-6604).

Thus, there is a need for novel engineered Fcγ sequences that have no or minimal binding affinity to FcγR and C1q but retain other properties of importance to a pharmaceutical product, such as pharmacokinetic profile or manufacturability.

### SUMMARY

Provided herein are compositions, comprising modified immunoglobulin constant domains useful in engineering of antibody or antibody-like therapeutics, such as those comprising an Fc region. Also described are related polynucleotides capable of encoding the provided modified constant domains, cells expressing the provided modified constant domains, as well as associated vectors. In addition, methods of using the provided modified constant domains are described.

The composition described is an IgG1 Fc mutant exhibiting diminished FcγR binding capacity but having conserved FcRn binding. These IgG Fc mutants enable therapeutic targeting of soluble or cell surface antigens while minimizing Fc-associated engagement of immune effector cells and complement mediated cytotoxicity. In one aspect, the IgG1 Fc-containing molecule comprises a CH2 domain in which amino acids at position 265, 297, and 329 indicated by the EU index as in Kabat, et al. are replaced by other amino acids.

In one embodiment, the amino acid at position 265 of the IgG1 Fc-containing molecule is replaced with alanine (A), asparagine (N) or glutamic acid (E), the amino acid at position 297 is replaced with alanine, aspartic acid (D), or glutamine (Q), and the amino acid at position 329 is replaced with alanine, glycine (G), or serine (S).

In certain embodiments, the IgG1 Fc mutant compositions are used in indications where retention of therapeutic antibody (or Fc-fusion) half-life is conserved through interactions with FcRn, while undesired effects derived from binding and/or activation of C1q and FcγRs associated with immune cells and effector functions such as i) antibody dependent cytotoxicity (ADCC), ii) complement dependent cytotoxicity (CDC), iii) antibody dependent cellular phagocytosis (ADCP), iv) FcγR-mediated cellular activation, v) FcγR-mediated platelet activation/depletion, and/or vi) FcγR-mediated cross-linking of the bound target, are minimized or eliminated.

In one aspect, the IgG1 Fc mutations are incorporated into therapeutic antibodies or Fc-fusions of binders, such as multivalent binders, targeting ligands on cells involved in cancer, neurological disorders, immune system disorders such as those related to B-cell or T-cell activation, or on cells involved in tissue repair or healing, such as fibroblasts or stem cells.

In certain embodiments, the IgG1 Fc mutant is comprised in a pharmaceutical composition. In certain embodiments, the IgG1 Fc mutant is part of a pharmaceutically active molecule. The pharmaceutical compositions comprising the IgG1 Fc mutant or active IgG1 Fc mutant-comprising molecules are useful for the treatment of diseases or disorders, for example cancer.

Also provided herein are recombinant IgG1 Fc-containing molecules having decreased affinity for C1q and to at least one Fcγ receptor (FcγR) as compared to an Fc-containing molecule with a wild type Fc domain, the recombinant IgG1 Fc-containing molecules comprising mutations at amino acid position 265, 297, and 329, wherein residue numbering is as indicated by the EU index as in Kabat, et al.

Further provided herein are recombinant polypeptides comprising (a) one or more binding domains capable of binding at least one target molecule; and (b) an IgG1 Fc domain comprising mutations at amino acid position 265, 297, and 329, wherein the polypeptide is capable of binding the target molecule without triggering significant Fcγ-mediated effects, such as complement dependent lysis, cell mediated destruction of the target molecule, and/or FcγR-mediated cross-linking of the bound target.

### BRIEF DESCRIPTION OF DRAWINGS

FIG. 1: Size exclusion chromatography profiles of mAb1 IgG1 and mAb1 DANAPA IgG1.

FIG. 2: Binding of anti-CD3 antibody mAb1 mutants with mutated Fc to CD3⁺ Jurkat cells.

FIGS. 3A and 3B: A) Induction of lymphocyte activation in human PBMC by mAb1 mutants with mutated Fcγ1 determined by CD69 surface staining. The dotted line represents the percentage CD69 positive cells obtained in the positive control wells containing CD2/CD3/CD28 activation beads. B) Induction of cytokine release in human PBMC by mAb1 mutants with mutated Fcγ1, as determined by IFNγ ELISA. The dotted line represents the level of IFNγ obtained in the positive control wells containing CD2/CD3/CD28 activation beads.

FIG. 4A-4D: A) Schematic illustration of the AlphaScreen™ Fc receptor competition binding assay. B) Binding of Fc mutated mAb1 mutants to human FcγRI, FcγRIIA, FcγRIIB and FcγRIIIA, analyzed by AlphaScreen™ Fc receptor competition binding assay. C,D) Binding of Fc mutated mAb1 mutants to human FcγRI (C) and human FcγRIIIA (D), analyzed by surface plasmon resonance (BIAcore).

FIG. 5: Binding of different antibodies in DANAPA IgG1 format to human FcγRI, FcγRIIA, FcγRIIB and FcγRIIIA, analyzed by AlphaScreen™ Fc receptor competition binding assay.

FIG. 6: Binding of mAb1 with different substitutions at position D265, N297 and P329 to human FcγRI, analyzed by AlphaScreen™ Fc receptor competition binding assay.

FIG. 7: Binding of mAb1 DANAPA IgG1 to human C1q, analyzed by surface plasmon resonance (BIAcore).

FIG. 8: Binding of mAb1 DANAPA IgG1 to human FcRn at pH 6.0, analyzed by surface plasmon resonance (BIAcore).

FIG. 9: Antibody plasma concentrations in C57BL/6 mice after i.v. administration of 10 mg/kg mAb1 DANAPA IgG1 or mAb1 IgG1, respectively. Data is presented as mean±standard deviation (n=5)

FIG. 10: Binding of different CD3/CD33 FynomAbs to human FcγRI, FcγRIIA, FcγRIIB and FcγRIIIA, analyzed by AlphaScreen™ Fc receptor competition binding assay.

### DETAILED DESCRIPTION OF ILLUSTRATIVE EMBODIMENTS

In order that the application may be more completely understood, several definitions are set forth below. Such definitions are meant to encompass grammatical equivalents.

Throughout the present specification and claims, the numbering of the residues in the Fc region is that of the immunoglobulin heavy chain according to the EU index as in Kabat et al., Sequences of Proteins of Immunological Interest, 5th Ed. Public Health Service, National Institutes of Health, Bethesda, Md. (1991), expressly incorporated herein by reference. The “EU index as in Kabat” herein refers to the residue numbering of the human IgG1 EU antibody. This numbering is well known to the skilled person and often used in the field.

By “polypeptide” or “protein” as used herein is meant at least two covalently attached amino acids, which includes proteins, polypeptides, oligopeptides and peptides.

By “amino acid” as used herein is meant one of the 20 naturally occurring amino acids or any non-natural analogues that may be present at a specific, defined position.

An “Fc-containing molecule having a substitution (or ‘mutation’, or ‘replacement’) at positions 265, 297 and 329”, means a molecule wherein the amino acid at position 265 is different from aspartic acid (D), the amino acid at position 297 is different from asparagine (N), and the amino acid at position 329 is different from proline (P), wherein all numbering in the Fc-region is according to the EU-index in Kabat et al.

“Amino acid changes”, herein include amino acid mutations such as substitution, insertion, and/or deletion in a polypeptide sequence. By “amino acid substitution” or “substitution” herein is meant the replacement of an amino acid at a particular position in a parent polypeptide sequence with another amino acid. For example, the substitution P329A refers to a mutant polypeptide, in this case an Fc mutant, in which the proline at position 329 is replaced with alanine.

In case of a combination of amino acid mutations, the preferred format is the following: D265A/N297A/P329A. That means that there are three amino acid mutations in the Fc region of the mutant as compared to its parent polypeptide: one in position 265 (aspartic acid (D) replaced with alanine (A)), one in position 297 (asparagine (N) replaced with alanine), and one in position 329 (proline (P) replaced with alanine).

The term “antibody” is used herein in the broadest sense. “Antibody” refers to any polypeptide which at least comprises (i) an Fc region and (ii) a binding polypeptide domain derived from a variable region of an immunoglobulin. Antibodies thus include, but are not limited to, full-length immunoglobulins, multi-specific antibodies, Fc-fusion protein comprising at least one variable region, synthetic antibodies (sometimes referred to herein as “antibody mimetics”), chimeric antibodies, humanized antibodies, fully human antibodies, heterodimeric antibodies, antibody-fusion proteins, antibody conjugates and fragments of each respectively. A “FynomAb” as described in more detail below also comprises an antibody.

By “full-length antibody” or by “immunoglobulin” as used herein is meant the structure that constitutes the natural biological form of an antibody, including variable and constant regions. “Full length antibody” covers monoclonal full-length antibodies, wild-type full-length antibodies, chimeric full-length antibodies, humanized full-length antibodies, fully human full-length antibodies, the list not being limitative.

In most mammals, including humans and mice, the structure of full-length antibodies is generally a tetramer. Said tetramer is composed of two identical pairs of polypeptide chains, each pair having one “light” chain (typically having a molecular weight of about 25 kDa) and one “heavy” chain (typically having a molecular weight of about 50-70 kDa). In some mammals, for example in camels and llamas, full-length antibodies may consist of only two heavy chains, each heavy chain comprising a variable domain attached to the Fc region.

The amino-terminal portion of each chain includes a variable region of about 100 to 110 or more amino acids primarily responsible for antigen recognition and comprising the so-called complementarity-determining regions (CDR).

The carboxy-terminal portion of each chain defines a constant region normally primarily responsible for effector functions.

In the case of human immunoglobulins, light chains are classified as kappa and lambda light chains. Heavy chains are classified as mu, delta, gamma, alpha, or epsilon, and define the antibody's isotype as IgM, IgD, IgG, IgA, and IgE, respectively.

As used herein, “human” antibodies include antibodies having the amino acid sequence of a human immunoglobulin and includes antibodies isolated from human immunoglobulin libraries or from animals transgenic for one or more human immunoglobulin and that do not express endogenous immunoglobulins.

By “IgG” as used herein is meant a polypeptide belonging to the class of antibodies that are substantially encoded by a recognized immunoglobulin gamma gene. In humans, IgG comprises the subclasses or isotypes IgG1, IgG2, IgG3, and IgG4. In mice, IgG comprises IgG1, IgG2a, IgG2b, IgG3. Full-length IgGs consist of two identical pairs of two immunoglobulin chains, each pair having one light and one heavy chain, each light chain comprising immunoglobulin domains VL and CL, and each heavy chain comprising immunoglobulin domains VH, Cγ1 (also called CH1), Cγ2 (also called CH2), and Cγ3 (also called CH3). In the context of human IgG1, “CH1” refers to positions 118-215, CH2 domain refers to positions 231-340 and CH3 domain refers to positions 341-447 according to the EU index as in Kabat. IgG1 also comprises a hinge domain which refers to positions 216-230 in the case of IgG1.

By “Fc” or “Fc region”, as used herein is meant the constant region of a full-length immunoglobulin excluding the first constant region immunoglobulin domain. Thus Fc refers to the last two constant region immunoglobulin domains of IgA, IgD, and IgG, the last three constant region immunoglobulin domains of IgE and IgM, and the flexible hinge N-terminal to these domains. For IgA and IgM, Fc may include the J chain. For IgG, Fc comprises immunoglobulin domains CH2, CH3 and the lower hinge region between CH1 and CH2. The Fc region of IgG1 comprises the domain from amino acid C226 to the carboxyl terminus end, wherein the numbering is according to the EU index as in Kabat. For example, the “Fc” or “Fc region” may include, without being limited to, Fc region of IgG1 comprising any one of the sequences SEQ ID NO: 43 and 52-58 (each of which are examples of human wild-type IgG1 Fc amino acid sequences), or comprising a sequence that is at least 80%, at least 85%, preferably at least 90%, at least 91%, at least 92%, at least 93%, at least 94%, more preferably at least 95%, at least 96%, at least 97%, at least 98%, or at least 99%, identical to SEQ ID NO: 43 or to any of SEQ ID NO: 52-58. In preferred embodiments, an Fc region according to the invention from position 226 (Kabat numbering) onwards comprises a sequence that is at least 80%, at least 85%, preferably at least 90%, at least 91%, at least 92%, at least 93%, at least 94%, more preferably at least 95%, at least 96%, at least 97%, or at least 98%, identical to SEQ ID NO: 43, and wherein the amino acid at position 265 is different from aspartic acid (D), the amino acid at position 297 is different from asparagine (N), and the amino acid at position 329 is different from proline (P). The analogous domains for other IgG sub-classes can be determined from amino acid sequence alignment of heavy chains or heavy chain fragments of said IgG sub-classes with that of human IgG1.

A “CH2 domain” as used herein is preferably of an Fc region of human IgG1, and comprises an amino acid sequence at least 80%, 85%, 90%, preferably at least 95%, at least 96%, at least 97%, at least 98%, at least 99%, or 100%, identical to SEQ ID NO: 60. A “CH3 domain” of an Fc region of human IgG1 as described herein comprises an amino acid sequence at least 80%, 85%, 90%, preferably at least 95%, at least 98%, or 100%, identical to SEQ ID NO: 61.

By “Fc-containing molecule” as used herein is meant a polypeptide that comprises an Fc region. Fc-containing molecules include, but are not limited to, antibodies, Fc fusions, isolated Fcs, Fc-conjugates, antibody fusions, FynomAbs, and the like.

By “wild-type” or “WT” herein is meant an amino acid sequence or a nucleotide sequence that is found in nature i.e. that is naturally-occurring, including allelic variations. A WT protein, polypeptide, antibody, immunoglobulin, IgG, etc. have an amino acid sequence or a nucleotide sequence that has not been intentionally modified by molecular biological techniques such as mutagenesis. For example, “wild-type Fc regions” may include, without being limited to, Fc region of IgG1 comprising the sequence SEQ ID NO: 43, which is an example of a human wild-type IgG1 Fc amino acid sequence, or Fc region of IgG comprising any one of the sequences of SEQ ID NOs: 52-58, each of which are also examples of human wild-type IgG1 Fc amino acid sequences.

The terms “Fc receptor” or “FcR” are used to describe a receptor that binds to an Fc region (e.g., the Fc region of an antibody).

The terms “Fc gamma receptor”, “Fcγ receptor” or “FcγR” refer to human receptors which bind the Fc region of IgG antibodies. As used herein, FcγR includes FcγRI (CD64), FcγRII (CD32), FcγRIII (CD16) subclasses including their allelic mutants and alternatively spliced forms of these receptors.

These FcγRs are also defined as either activating receptors (FcγRI, FcγRIIa/c, FcγRIIIa/b) or inhibitory receptor (FcγRIIb) as they elicit or inhibit immune functions.

FcγRI family is composed of three genes (FCGRIA, FCGRIB and FCGRIC) but only the product of FCGRIA has been identified as full-length surface receptor. The said product, namely FcγRI, is expressed by dendritic cells (DC), macrophages and also activated neutrophils.

FcγRII family is composed of three genes (FCGR2A, FCGR2B and FCGR2C) which encode the FcγRIIa, FcγRIIb and FcγRIIc proteins. FcγRIIa is expressed on monocytes, certain dendritic cells and neutrophils. FcγRIIc is expressed on natural killer (NK) cells. FcγRIIb is the broadly expressed FcγR. FcγRIIb is virtually present on all leukocytes with exception of NK cells and T cells.

FcγRIII family is composed of two genes FCGR3A and FCGR3B which encode FcγRIIIa and FcγRIIIb. The FcγRIIIa protein is expressed as a transmembrane protein on monocytes, tissue specific macrophages, dendritic cells, γδ T cells, and natural killer cells. FcγRIIIb is a GPI-anchored receptor expressed on the surface of neutrophils and basophils.

Two alleles of the gene encoding FcγRIIa generate 2 mutants differing at position 131 (low-responder FcγRIIaR131 and high-responder FcγRIIaH131). Similarly, two alleles of the gene encoding FcγRIIIa generate 2 mutants differing at position 158 (low-responder FcγRIIIaF158 and high-responder FcγRIIIaV158).

Noticeably, NK cells, which are believed to be the crucial mediators of antibody-dependent cell-cytotoxicity, only express FcγRIIIa and FcγRIIc and none of the other FcγRs, in particular, the inhibitory FcγRIIb.

Each FcγR protein has differential ligand binding preferences with respect to IgG subclasses and distinct affinities for IgG subclasses.

Activating FcγRs trigger various immune responses such as phagocytosis, respiratory burst and cytokine production (TNF-α, IL-6) by antigen presenting cells (APC), antibody-dependent cellular cytotoxicity (ADCC) and degranulation by neutrophils and NK cells. Activating FcγRs also play an important role in the clearance of immune complex. On the other hand, the inhibitory receptor FcγRIIb is a critical regulatory element in B-cell homeostasis. It controls the threshold and the extent of cell activation.

Fc gamma receptors and their functions are reviewed in Nimmerjahn and Ravetch, Nature Reviews Immunology, 2008, 8, 34-47.

As used herein, “C1q” is a hexavalent molecule with a molecular weight of approximately 460,000 and a structure likened to a bouquet of tulips in which six collagenous “stalks” are connected to six globular head regions. C1q forms with the two serine proteases, C1r and C1s, the complex C1 which is the first component of the complement cascade pathway.

C1q and its function are reviewed e.g. in Kishore et al., Immunopharmacology, 2000, 49:159-170 and Sjoberg et al. Trends Immunol. 2009 30(2):83-90.

By “FcRn” or “neonatal Fc Receptor” as used herein is meant a protein that binds the IgG antibody Fc region and is encoded at least in part by an FCRN gene. As is known in the art, the functional FcRn protein comprises two polypeptides, often referred to as the heavy chain and light chain. The light chain is beta-2-microglobulin and the heavy chain is encoded by the FCRN gene. FcRn or FcRn protein refers to the complex of α-chain with beta-2-microglobulin. In human, the gene coding for FcRn is called FCGRT. FcRn is involved in the transfer of passive humoral immunity from a mother to her fetus and also in the control of the clearance of IgGs.

FcRn and its function is reviewed e.g. in Roopenian, Nature Reviews Immunology, 2007, 7, 715-725.

A molecule “retains binding to FcRn” as used herein when it binds to FcRn with a KD that is lower than 5-fold, preferably lower than 4-fold, more preferably lower than 3-fold, still more preferably lower than 2-fold the KD, of the parental Fc-containing molecule without the amino acid substitution (e.g. wild-type IgG1), as measured using surface plasmon resonance (SPR), wherein the KD is measured at pH 6.0. In certain embodiments the KD is about 1 to 2-fold, e.g. about 1.5-fold the KD of the parental molecule, or about the same as (i.e. 1-fold) the KD of the parental molecule, and in certain embodiments the KD can also be lower than the KD of the parental molecule.

“Reduced binding” refers to reduced binding of the Fc-containing molecules of the invention having at least one amino acid substitution in the Fc region described herein, for instance to C1q and/or to FcγR receptor when compared to the binding of the parental Fc-containing molecule without the amino acid substitution. “Reduced binding” may be at least about 2-fold, at least about 5-fold, at least about 10-fold, at least about 20-fold, at least about 50-fold, at least about 75-fold, or at least about 100-fold reduced binding. Binding of Fc-containing molecules can be assayed using a variety of techniques known in the art, including but not limited to surface plasmon resonance (SPR). SPR measurements can be performed using a BIAcore@ instrument. In practice, Fc-containing molecules exhibiting “reduced binding” to a particular FcγR refer to Fc-containing molecules that have significantly reduced or abrogated effector function mediated by the particular FcγR.

“Recombinant” as used herein, includes antibodies and other proteins that are prepared, expressed, created or isolated by recombinant means.

“Vector” means a polynucleotide capable of being duplicated within a biological system or that can be moved between such systems. Vector polynucleotides typically contain elements, such as origins of replication, polyadenylation signal or selection markers, that function to facilitate the duplication or maintenance of these polynucleotides in a biological system. Examples of such biological systems may include a cell, virus, animal, plant, and reconstituted biological systems utilizing biological components capable of duplicating a vector. The polynucleotide comprising a vector may be DNA or RNA molecules or a hybrid of these.

“Polynucleotide” means a molecule comprising a chain of nucleotides covalently linked by a sugar-phosphate backbone or other equivalent covalent chemistry. Double and single-stranded DNA and RNA are typical examples of polynucleotides.

Fc Mutants with Decreased Binding to C1q and FcγRs

The present invention is a demonstration for the first time of combined substitutions in positions 265, 297, and 329 of the IgG1 constant regions (Fc), according to the EU index as in Kabat. The directed selection of multiple residue substitutions unexpectedly provided a functional Fc domain for use in antibody engineering and for use as a fusion polypeptide as well as the possibility of providing a therapeutic entity which is devoid of measurable effector function.

The invention thus provides a recombinant IgG1 Fc-containing molecule, comprising a CH2 domain in which amino acid D at position 265, amino acid N at position 297, and amino acid P at position 329 indicated by the EU index as in Kabat are replaced by other amino acids.

Preferred IgG1 Fc-containing molecules include, but are not limited to, those comprising an amino acid substitution at position 265, 297 and 329. As discussed below, such polypeptides may have one or more additional deletions, additions, or substitutions within the Fc region. Thus, within the scope of the invention are IgG1 Fc-containing molecules having an amino acid substitution at at position 265 (i.e. having an amino acid different from D at this position), 297 (i.e. having an amino acid different from N at this position) and 329 (i.e. having an amino acid different from P at this position) and at the same time the Fc-regions from position 226 (Kabat numbering) onwards are at least 80%, at least 85%, preferably at least 90%, at least 91%, at least 92%, at least 93%, at least 94%, more preferably at least 95%, at least 96%, at least 97%, or at least 98% identical to SEQ ID NO: 43.

The term “percent (%) sequence identity” or “% identity” describes the number of matches (“hits”) of identical amino acids of two or more aligned amino acid sequences as compared to the number of amino acid residues making up the overall length of the amino acid sequences. In other terms, using an alignment, for two or more sequences the percentage of amino acid residues that are the same (e.g. 90%, 95%, 97% or 98% identity) may be determined, when the sequences are compared and aligned for maximum correspondence as measured using a sequence comparison algorithm as known in the art, or when manually aligned and visually inspected. The sequences which are compared to determine sequence identity may thus differ by substitution(s), addition(s) or deletion(s) of amino acids. Suitable programs for aligning protein sequences are known to the skilled person. The percentage sequence identity of protein sequences can, for example, be determined with programs such as CLUSTALW, Clustal Omega, FASTA or BLAST, e.g using the NCBI BLAST algorithm (Altschul S F, et al (1997), Nucleic Acids Res. 25:3389-3402).

For example, for amino acid sequences, sequence identity and/or similarity can be determined by using standard techniques known in the art, including, but not limited to, the local sequence identity algorithm of Smith and Waterman, 1981, Adv. Appl. Math. 2:482, the sequence identity alignment algorithm of Needleman and Wunsch, 1970, J. Mol. Biol. 48:443, the search for similarity method of Pearson and Lipman, 1988, Proc. Nat. Acad. Sci. U.S.A. 85:2444, computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al, 1984, Nucl. Acid Res. 12:387-395, preferably using the default settings, or by inspection. In certain embodiments, percent identity is calculated by FastDB based upon the following parameters: mismatch penalty of 1; gap penalty of 1; gap size penalty of 0.33; and joining penalty of 30, “Current Methods in Sequence Comparison and Analysis,” Macromolecule Sequencing and Synthesis, Selected Methods and Applications, pp 127-149 (1988), Alan R. Liss, Inc.

Another example of a useful algorithm is PILEUP. PILEUP creates a multiple sequence alignment from a group of related sequences using progressive, pairwise alignments. It can also plot a tree showing the clustering relationships used to create the alignment. Useful PILEUP parameters including a default gap weight of 3.00, a default gap length weight of 0.10, and weighted end gaps.

Another example of a useful algorithm is the BLAST algorithm, described in: Altschul et al, 1990, J. Mol. Biol. 215:403-410; Altschul et al, 1997, Nucleic Acids Res. 25:3389-3402; and Karin et al, 1993, Proc. Natl. Acad. Sci. U.S.A. 90:5873-5787. A particularly useful BLAST program is the WU-BLAST-2 program which was obtained from Altschul et al, 1996, Methods in Enzymology 266:460-480. WU-BLAST-2 uses several search parameters, most of which are set to the default values.

An additional useful algorithm is gapped BLAST as reported by Altschul et al, 1993, Nucl. Acids Res. 25:3389-3402.

The multi-substituted IgG1 mutants were selected on the basis of their relative affinities for human FcRs (FcγRI, FcγRIIa, FcγRIIb, FcγRIIIa and FcRn) assessed by AlphaScreen™ competition assays and SPR/Biacore analyses. These mutants were further tested and ranked in the appropriate cellular systems for their ability to induce cytokine release by PBMCs. In the set of experimental data provided herein, the IgG1 Fc mutants were compared to wild-type IgG1 Fc-containing molecules. Further analyses of these mutants in several in vitro bioassays demonstrated minimal to undetectable levels of activity and greatly ablated binding affinity for FcγRs. Based on these screens, IgG1 Fc mutants, comprising substitutions at all three amino acid positions 265, 297 and 329 combined, were surprisingly identified to have no or minimal detectable affinity for FcγRs and are virtually or completely devoid of activity in the various aforementioned effector/immunostimulatory bioassays. The IgG1 Fc mutants of the invention may be considered a truly “silenced” Fc in having no or minimal ability to bind FcγRs, mediate effector functions, or engage Fc-mediated cytokine release.

Based on the present invention, substitutions at amino acid positions 265, 297 and 329 can optionally be combined with other amino acid mutations, or the substitutions can be used in another IgG isotype to achieve similar or selective silencing of effector functions as taught herein and combined with what is known in the art. This combination of mutations at positions 265, 297 and 329 surprisingly led to significantly improved silencing compared to previously described Fc mutation N297A, or Fc double mutation L234A/L235A, each of which have been used in clinical-stage therapeutic antibodies/Fc-containing proteins for which minimal residual FcγR interaction is desired (Herold K C, et al. (2005). Diabetes 54(6): 1763-1769).

The D265, N297 and P329 triple mutant according to the present invention exhibits a reduced binding to the first complement component C1q as compared to its wild-type counterpart. In other words, the affinity of the mutant for C1q is lower than that of the wild-type.

The D265, N297 and P329 triple mutant according to the present invention also exhibits an affinity for at least one Fcγ receptor lower than that of its parent polypeptide. As used herein, Fcγ receptors include FcγRI, FcγRII and FcγRIII receptors. Preferably, the at least one FcγR is selected from the group consisting of FcγRI, FcγRIIa, FcγRIIb, FcγRIIIa.

The D265, N297 and P329 triple mutant exhibits a reduced binding to both C1q and Fcγ receptors as compared to its wild-type counterpart.

In certain embodiments, the mutant IgG1 Fc-containing molecule exhibits a reduced binding to C1q, FcγRI, FcγRIIa, FcγRIIb, and FcγRIIIa as compared to its wild-type counterpart.

The binding for C1q or for anyone of Fc receptors can be evaluated by well-known methods of the prior art such as AlphaScreen™ and Surface Plamon Resonance (SPR).

For example, the bond strength of a mutant of the invention for a protein of interest (such as C1q or a FcγR) may be compared to that of its wild-type counterpart by calculating the ratio of their specific IC₅₀ values obtained by AlphaScreen™ competition assay as described in Example 4. AlphaScreen™, used in high throughput screening, is a homogenous assay technology which allows detection of molecular events such as binding. Coated “Donor” and “Acceptor” beads are the basis of the assay technology. As a bead based assay, AlphaScreen™ works through the interaction of the beads in close proximity, resulting in a cascade of chemical reactions that act to produce a greatly amplified signal. Direct or indirect, e.g., competitive binding, measurements can be applied for assessing relative affinities and avidities among and between proteins.

As an alternative, the binding of the mutant IgG1 Fc-containing molecule and that of its wild-type counterpart for a protein of interest (e.g., C1q and/or an FcγR) may be compared through the determination of EC₅₀ by an appropriate ELISA assay. The EC₅₀ refers to the concentration of the mutant which provides a signal representing 50% of the saturation of the curve relating to the percentage of bound protein of interest versus the log of the concentration of the mutant. Generally, a mutant IgG1 Fc-containing molecule is considered to display a reduced binding to a protein of interest (such as C1q and/or an FcγR) as compared to its wild-type counterpart if its EC₅₀ is at least 1.5-fold higher than that of its wild-type counterpart.

The binding affinity of the mutant IgG1 Fc-containing molecule to a protein of interest (e.g., C1q and/or a FcγR) may also be assessed by SPR through the determination of the constant of dissociation (Kd). Generally, a mutant IgG1 Fc-containing molecule is considered to display a reduced binding to a protein of interest (e.g. C1q and/or a FcγR) as compared to its wild-type counterpart if its Kd is at least 1.5-fold higher than that of its polypeptide parent.

The affinity of the mutant for C1q or for an FcγR may be so weak that the specific signal by AlphaScreen™ assay and even the Kd by SPR or the EC₅₀ by ELISA assay cannot be accurately determined since the binding signal is in the background noise or under the threshold of detection. In such a case, the mutant IgG1 Fc-containing molecule is considered not to bind the C1q and/or respective FcγR.

For example, the triple mutant IgG1 Fc-containing molecule according to the invention may not bind to at least one FcγR and exhibits a reduced or no binding to C1q. Such a mutant IgG1 Fc-containing molecule is clearly illustrated in the examples of the present application.

In some embodiments, the mutant IgG1 Fc-containing molecule of the invention does not bind to at least one protein selected from C1q and Fcγ receptors.

The Applicant showed that the introduction of mutations at D265, N297 and P329 are sufficient to significantly impair the binding to C1q and to Fcγ receptors. In other words, no mutation other than those at D265, N297 and P329 needs to be introduced within the IgG1 Fc region of the IgG1 wild-type counterpart in order to obtain a mutant IgG1 Fc-containing molecule with appropriate reduced binding to C1q and/or Fcγ receptors. Nevertheless, it would optionally be possible to add further mutations to the Fc-containing molecule of the invention if so desired, e.g. to alter other functionalities of the molecule.

Without to be bound by any theory, the Applicant believes that the amino acid substitutions provided by the present invention do not significantly cause major structural rearrangement in the IgG1 Fc region so that in some cases, the other functions which are not mediated by the binding to C1q and FcγRs are not significantly altered as compared to those of the polypeptide parent. Noticeably, the Applicant showed that the introduction of substitution mutations at positions D265, N297 and P329 in the IgG1 Fc region does not significantly impair their affinity for neonatal Fc Receptor (FcRn). For example, the dissociation constant, K_{D}, for mAb1, comprising D265A, N297A and P329A IgG1 Fc substitutions (DANAPA), is 500 nM and 470 nM for its wild-type counterpart (see Example 8). In other words, the wild-type IgG1 Fc-containing molecule and mutant IgG1 Fc-containing molecule according to the present invention display close binding property for FcRn.

As mentioned hereabove, the Fc region of the wild-type may be selected from the group consisting of wild-type Fc regions of human IgGs, fragments and mutants thereof.

As indicated hereabove, the Fc region of the invention may comprise amino acid substitutions of at least three amino acids in the IgG1 Fc. For reminder, wild-type Fc regions include, without being limited to, the Fc region of human IgG1 having SEQ ID NO: 43. Allelic variants of human Fc regions are known and can also be used as the parent molecule to introduce the combination of mutations according to the invention. Allelic variants of human IgG1 Fc differ from each other at position 356 (Glutamic acid (E) or Aspartic acid (D)), and/or at position 358 (Methionine (M) or Leucine (L)) and/or at position 431 (Alanine (A) or Glycine (G)). Allelic variants include naturally occurring allelic variants as well as non-natural allelic variants. Non-natural allelic variants contain residues which do occur in naturally occurring allelic variants but in combinations which are not found in nature. Jefferis et al. provide an overview on human IgG allelic variants which allows a skilled person to derive naturally occurring and non-natural allelic variants of Fc sequences (Jefferis R and M-P Lefranc (2009) mAbs 1: 1-7). In certain embodiments, the parent molecule for the introduction of the combination of mutations according to the invention (i.e. mutations at positions 265, 297 and 329 according to Kabat numbering) therefore is a molecule comprising a human IgG Fc sequence chosen from the group consisting of SEQ ID NOs: 43, 52, 53, 54, 55, 56, 57, and 58. The invention in specific embodiments thus provides recombinant IgG1 Fc-containing polypeptides comprising an amino acid sequence according to any one of SEQ ID Nos: 43, 52, 53, 54, 55, 56, 57, and 58, characterized in that: (i) the amino acid D at position 265 has been replaced by another amino acid, (ii) the amino acid N at position 297 has been replaced by another amino acid, and (iii) the amino acid P at position 329 has been replaced by another amino acid, wherein the numbering is indicated by the EU index as in Kabat.

An Fc region according to the invention as compared to a wild-type or parent Fc region has a combination of mutations, such that amino acid residues at positions 265, 297 and 329 are different from D, N and P, respectively, wherein numbering is according to the EU index in Kabat et al. In certain embodiments, the amino acid residue at position 265 is A, N or E. In certain embodiments, the amino acid residue at position 297 is A, D or Q. In certain embodiments, the amino acid residue at position 329 is A, G or S. The skilled person will appreciate that other amino acids can be substituted on these positions (e.g. R, C, Q, G, H, I, L, K, M, F, P, S, T, W, Y, or V at position 265; R, C, E, G, H, I, L, K, M, F, P, S, T, W, Y, or V at position 297; R, C, Q, N, H, I, L, K, M, F, E, D, T, W, Y, or V at position 329) and resulting Fc variants having the indicated amino acids at positions 265, 297 and 329 can be tested by routine methods for having substantially the same properties in binding to Fc receptors and C1q as the embodiments exemplified in the working examples herein, and such variants are included in the present invention.

In some embodiments, the amino acid substitutions of the IgG1 Fc-containing molecule comprise amino acid substitutions D265A, N297A, P329A.

In some other embodiments, the amino acid substitutions of the IgG1 Fc-containing molecule comprise amino acid substitutions D265N, N297D, P329G.

In some other embodiments, the amino acid substitutions of the IgG1 Fc-containing molecule comprise amino acid substitutions D265E, N297Q, P329S.

In a specific embodiment, a mutant IgG1 Fc-containing molecule, which mutant exhibits reduced binding to the protein C1q and to at least one receptor FcγR as compared to the wild-type IgG1 Fc-containing molecule is characterized in that:
1. amino acid at position 265 is replaced with alanine, asparagine or glutamic acid,
2. amino acid at position 297 is replaced with alanine, aspartate, or glutamine, and
3. amino acid at position 329 is replaced with alanine, glycine, or serine, wherein the numbering of amino acids in indicated by the EU index as in Kabat.

In some embodiments, a method of making a recombinant IgG1 Fc-containing molecule, comprising a CH2 domain in which amino acids at position 265, 297, and 329 indicated by the EU index as in Kabat are replaced by other amino acids than D, N and P respectively, comprises the steps of:

(a) providing a nucleic acid encoding a parent IgG1 Fc-containing molecule,

(b) modifying the nucleic acid provided in step (a) so as to obtain a nucleic acid encoding a recombinant IgG1 Fc-containing molecule wherein the amino acids at at least one of positions 265, 297, and 329 are replaced such that in the resulting encoded Fc-containing molecule the amino acids on these positions are different from D (position 265), N (position 297) and P (position 329), and

(c) expressing the nucleic acid obtaining in step (b) in a host cell and recovering the said mutant.

Of course, if the parent molecule already contains a different amino acid than D at position 265, N at position 297, or P at position 329, only the other one or two of these three positions still needs to be modified to create an Fc containing molecule according to the invention.

Such steps may be performed by conventional practices of molecular biology. For carrying out a method of making a recombinant IgG1 Fc-containing molecule of the invention, the one skilled in the art may refer to well-known procedures described in the prior art which may be found e.g. in Molecular Cloning—A Laboratory Manual, 3rd Ed. (Maniatis, Cold Spring Harbor Laboratory Press, New York, 2001), The condensed protocols from Molecular cloning: a laboratory manual (Sambrook, Russell, CSHL Press, 2006), and Current Protocols in Molecular Biology (John Wiley & Sons, 2004).

The nucleic acid of the wild-type IgG1 Fc-containing molecule may be commercial or may be obtained by classical procedure of molecular biology or chemical synthesis. The nucleic acid encoding the mutant IgG1 Fc-containing molecule as mentioned in step (b) may be achieved by chemical synthesis, or by modifying the nucleic acid of the parent polypeptide using a variety of methods known in the prior art. These methods include, but are not limited to site-directed mutagenesis, random mutagenesis, PCR mutagenesis and cassette mutagenesis.

The nucleic acid encoding the mutant IgG1 Fc-containing molecule may be incorporated into an expression vector for its expression in a host cell.

Expression vectors typically include a protein encoding sequence operably linked, that is, placed in a functional relationship, with control or regulatory sequences such as a promoter, as well as optionally including selectable markers, any fusion partners, and/or additional elements. The mutant IgG1 Fc-containing molecule of the present invention may be produced by culturing a host cell transformed with nucleic acid, preferably an expression vector, containing nucleic acid encoding the mutant IgG1 Fc-containing molecule, under the appropriate conditions to induce or cause expression of the protein. A wide variety of appropriate host cell lines may be used, including but not limited to mammalian cells, bacteria, insect cells, and yeast.

For example, a variety of mammalian cell lines that can be used are described in the ATCC cell line catalog, available from the American Type Culture Collection. Host cells may be, but not limited to, YB2/0 (YB2/3HL.P2.GII.IGAg.20 cell, deposit to the American Type Culture Collection, ATCC n° CRL-1662), SP2/0, YE2/0, 1R983F, Namalwa, PER.C6, CHO cell lines, particularly CHO-K-1, CHO-Lecl0, CHO-Lecl, CHO-Lecl3, CHO Pro-5, CHO dhfr-, Wil-2, Jurkat, Vero, Molt-4, COS-7, HEK293, BHK, Vero, MDCK, immortalized amniotic cell lines (CAP), EB66, KGH6, NSO, SP2/0-Ag 14, P3X63Ag8.653, C127, JC, LA7, ZR-45-30, hTERT, NM2C5, UACC-812 and the like. The methods of introducing exogenous nucleic acid into host cells are well known in the art, and may vary with the host cell used.

The host cell may optionally belong to a transgenic non-human animal or to a transgenic plant. In this case, the mutant IgG1 Fc-containing molecule is thus obtained from a transgenic organism.

A transgenic non-human animal can be obtained by directly injecting a desired gene into a fertilized egg (Gordon et al., 1980 Proc Natl Acad Sci USA.; 77:7380-4). The transgenic non-human animals include mouse, rabbit, rat, goat, cow, cattle or fowl, and the like. A transgenic non-human animal having a desired gene can be obtained by introducing the desired gene into an embryonic stem cell and preparing the animal by an aggregation chimera method or injection chimera method (Manipulating the Mouse Embryo, A Laboratory Manual, second edition, Cold Spring Harbor Laboratory Press (1994); Gene Targeting, A Practical Approach, IRL Press at Oxford University Press (1993)). Examples of the embryonic stem cell include embryonic stem cells of mouse (Evans and Kaufman, 1981, Nature; 292:154-156), rat, goat, rabbit, monkey, fowl, cattle and the like. In addition, a transgenic non-human animal can also be prepared using a clonal technique in which a nucleus into which a desired gene is introduced is transplanted into an enucleated egg (Ryan et al., 1997 Science; 278: 873-876; Cibelli et al., 1998 Science, 280: 1256-1258). The mutant IgG1 Fc-containing molecule can be produced by introducing DNA encoding the mutant IgG1 Fc-containing molecule into an animal prepared by the above method to thereby form and accumulate the mutant molecule in the animal, and then collecting the mutant from the animal. The mutant IgG1 Fc-containing molecule may be made to be formed and accumulated in the milk, egg or the like of the animal.

In all the above cited embodiments, an IgG1 Fc-containing molecule may be a naturally occurring polypeptide (wild-type polypeptide), a mutant or an engineered version of a naturally occurring polypeptide, or a synthetic polypeptide.

In some embodiments, an IgG1 Fc-containing molecule is selected from the group consisting of IgG1 Fc-fusion protein, IgG1 Fc-conjugate, and antibodies.

As used herein, Fc-fusion protein and Fc-conjugate consist of an Fc region linked to a partner. The Fc region can be linked to its partner with or without a spacer, also referred to as linker.

Suitable linkers are at the skilled person's disposal. A linker can for instance be selected from the group consisting of alkyl with 1 to 30 carbon atoms, polyethylene glycol with 1 to 20 ethylene moieties, polyalanine with 1 to 20 residues, caproic acid, substituted or unsubstituted poly-p-phenylene and triazol. Preference is given to peptidic linkers, more specifically to oligopeptides having a length from 1 to 30 amino acids. Preferred length ranges are from 5 to 15 amino acids.

Particularly preferred are linkers which are peptides which consist of at least 50%, at least 60%, at least 70%, at least 80%, at least 90% or 100% of small amino acids such as glycine, serine and alanine. Particularly preferred are linkers consisting of glycines and serines only. A non-limiting example of a suitable linker is a (G4S)3 linker (SEQ ID NO: 40).

According to the present invention, an Fc fusion protein is a protein that comprises a protein, a polypeptide or a small peptide covalently linked to an Fc region. An Fc fusion protein optionally comprises a peptide linker as described above. Virtually any protein or small peptide may be linked to Fc regions to generate an Fc fusion. Protein fusion partners may include, but are not limited to, the target-binding region of a receptor, an adhesion molecule, a ligand, an enzyme, a cytokine, a chemokine, or some other protein or protein domain. Some non-limiting examples of Fc fusion proteins include alefacept, abatacept, belatacept, rilonacept, etanercept, romiplostim, and ablifercept.

In particular the Fc-fusion protein can be an immunoadhesin, i.e. antibody-like protein which combines the binding domain of a heterologous “adhesion” protein (i.e receptor, ligand or enzyme) with a fragment of immunoglobulin constant domain (i.e. an Fc region) (see for a review about immunoadhesins, Ashkenazi A, Chamow S M. 1997, Curr Opin Immunol. 9(2): 195-200).

Small peptides may include, but are not limited to, any therapeutic agent that directs the Fc fusion to a therapeutic target.

According to the present invention, an Fc conjugate may in certain embodiments result from the chemical coupling of an Fc region with a conjugate partner and optionally comprises a spacer linking the Fc region to the conjugate partner. The conjugate partner can be proteinaceous or non-proteinaceous. The coupling reaction generally uses functional groups on the Fc region and on the conjugate partner.

Suitable conjugate partners include, but are not limited to, therapeutic polypeptides, labels (for example of labels, see further below), drugs, cytotoxic agents, cytotoxic drugs (e.g., chemotherapeutic agents), toxins and active fragments of such toxins. Suitable toxins and their corresponding fragments include, but are not limited to, diptheria A chain, exotoxin A chain, ricin A chain, abrin A chain, and the like. A cytotoxic agent may be any radionuclide which can be directly conjugated to the IgG1 Fc mutant or sequestrated by a chelating agent which is covalently attached to the IgG1 Fc mutant. In additional embodiments, the conjugate partners can be selected from the group consisting of calicheamicin, auristatins, geldanamycin, maytansine, and duocarmycins and analogs.

In one embodiment, the IgG1 Fc-containing molecule comprises a “Fynomer”. Fynomers are small 7-kDa globular proteins derived from the SH3 domain of the human Fyn kinase (Fyn SH3, aa 83-145 of Fyn kinase: GVTLFVALYDYEARTEDDLSFHKGEKFQILNSSEGDWWEARSLTTGETGYIPS NYVAPVDSIQ (SEQ ID NO: 59). In SEQ ID NO: 59 as shown above the sequences of the RT and the src loop are underlined and double-underlined, respectively, and such molecules can be engineered to bind with antibody-like affinity and specificity to virtually any target of choice through random mutation of two loops (RT- and src-loop) on the surface of the Fyn SH3 domain, optionally combined with mutations of other selected positions in the Fyn SH3 domain (see, e.g. WO 2008/022759). Fyn SH3-derived polypeptides or Fynomers are well known in the art and have been described e.g. in Grabulovski et al. (2007) JBC, 282, p. 3196-3204; WO 2008/022759; Bertschinger et al (2007) Protein Eng Des Sel 20(2):57-68; and Gebauer and Skerra (2009) Curr Opinion in Chemical Biology 13:245-255. The term “Fyn SH3-derived polypeptide”, used interchangeably herein with the term “Fynomer”, refers to a non-immunoglobulin-derived binding polypeptide (e.g. a so-called scaffold as described in Gebauer and Skerra (2009) Curr Opinion in Chemical Biology 13:245-255) derived from the human Fyn SH3 domain. Fynomers can be genetically fused to other molecules such as antibodies, to create so-called FynomAbs that can be engineered to have dual specificity (e.g. Silacci et al, 2016, mAbs 8:1, 141-149; Brack et al, 2014, Mol Cancer Ther 13(8): p. 2030-9; WO 2014/044758 A1; WO 2014/170063 A1; WO 2015/141862 A1).

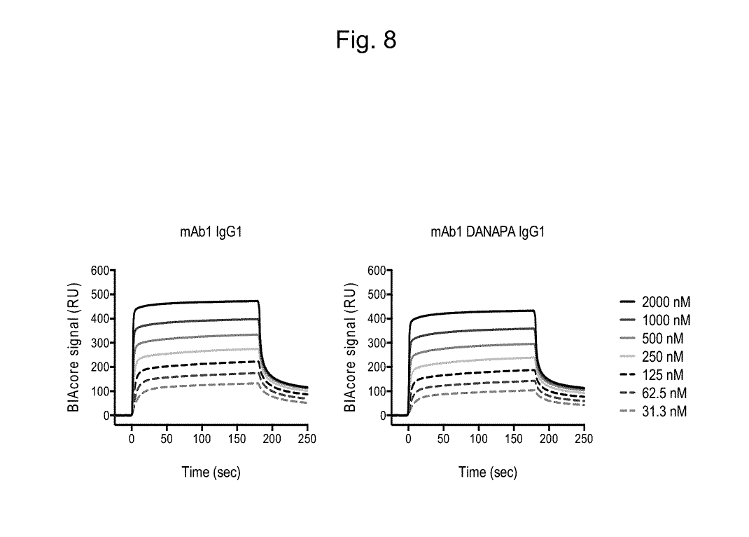

As mentioned, the term “antibody” is used herein in the broadest sense. According to the present invention, “antibody” refers to any polypeptide which at least comprises (i) an Fc region and (ii) a binding polypeptide domain derived from a variable domain of an immunoglobulin. The said binding polypeptide domain is able to bind specifically one given target antigen or a group of target antigens. A binding polypeptide domain which derives from a variable region of an immunoglobulin comprises at least one or more CDRs. Herein, antibodies include, but are not limited to, full-length antibodies, multi-specific antibodies, Fc-fusion protein comprising at least one variable region or synthetic antibodies (sometimes referred to herein as “antibody mimetics”), antibody-fusion proteins, antibody conjugates and fragments of each respectively. FynomAbs according to the invention also comprise antibodies with an Fc region. The invention thus also provides FynomAbs, i.e. one or more copies of a Fynomer coupled to an antibody, that comprise an Fc region with the mutations according to the invention, i.e. having an amino acid different from D at position 265, an amino acid different from N at position 297, and an amino acid different from P at position 329, wherein numbering is according to the EU index as in Kabat et al. The Fynomer can be covalently linked via a linker peptide to the antibody, or may be directly fused to the antibody. The Fynomer in certain embodiments may be located downstream of the C-terminus of the heavy chain of the antibody, or upstream of the N-terminus of the heavy chain of the antibody, or downstream of the C-terminus of the light chain of the antibody, or upstream of the N-terminus of the light chain of the antibody. Preferably, two copies of the Fynomer are coupled to the antibody, one of each to a corresponding terminus in two chains of the antibody, e.g. one copy at the N-terminus of the light chain of the first half of the antibody and one copy at the N-terminus of the light chain of the second half of the antibody (a “half” of an antibody meaning herein a heavy chain and a light chain that together comprise a binding region), or one copy at the N-terminus of the heavy chain of the first half of the antibody and one copy at the N-terminus of the heavy chain of the second half of the antibody, or one copy at the C-terminus of the light chain of the first half of the antibody and one copy at the C-terminus of the light chain of the second half of the antibody, or one copy at the C-terminus of the heavy chain of the first half of the antibody and one copy at the C-terminus of the heavy chain of the second half of the antibody (see e.g. Brack et al, 2014, Mol Cancer Ther 13: 2030-2039, and FIG. 8 of WO 2013/135588, for examples of different positions of Fynomers at the four termini of an IgG antibody). Such fusions can be generated by genetic engineering, cloning nucleic acid encoding the Fynomer part and the respective antibody chain in frame to form a single fusion molecule. Co-expression with the other chain of the antibody (e.g. the light chain in case the Fynomer is fused to the heavy chain, or the heavy chain in case the Fynomer is fused to the light chain) within a cell will lead to expression of functional Fynomabs. The Fynomer part may bind to a different target molecule than the antibody part (for non-limiting examples see e.g. Fynomabs described in Silacci et al, 2016, mAbs 8:1, 141-149; WO 2014/044758 A1; WO 2014/170063 A1; WO 2015/141862 A1) or the Fynomer part may bind to a different epitope on the same target molecule as the antibody part (for non-limiting examples see Fynomabs described in Brack et al, 2014, Mol Cancer Ther 13(8): p. 2030-9; WO 2013/135588).

By Fc-fusion protein comprising at least one variable region is meant an engineered protein comprising (i) an Fc region and (ii) a binding polypeptide domain derived from a variable domain of an immunoglobulin. Of particular interest are antibodies that comprise (a) an IgG Fc mutant of the invention, and (b) one of the following binding polypeptide domains derived from a variable region of an immunoglobulin (i.e. which comprise at least one CDR): (i) the Fab fragment consisting of VL, VH, CL and CH1 domains, (ii) the Fd fragment consisting of the VH and CH1 domains, (iii) the Fv fragment consisting of the VL and VH domains of a single antibody; (iv) isolated CDR regions, (v) F(ab′)2 fragments, a bivalent fragment comprising two linked Fab fragments (vi) single chain Fv molecules (scFv), wherein a VH domain and a VL domain are linked by a peptide linker which allows the two domains to associate to form an antigen binding site, (vii) bispecific single chain Fv and (viii) “diabodies” or “triabodies”, multivalent or multispecific fragments constructed by gene fusion, this list not being limitative.

By “full length antibody” herein is meant an antibody having the natural-occurring biological form of an antibody, including variable and constant regions. A full-length antibody may be a wild-type antibody, a mutant of a wild-type antibody (e.g. comprising pre-existing modifications), an engineered version of a wild-type antibody (e.g. for example a chimeric, a humanized antibody or a fully human antibody, see further below), this list not being limitative. As well-known, the structure of a full-length antibody is generally a tetramer except for some mammals such as llamas and camels in which some immunoglobulins are dimers.

The scaffold components of the full-length antibody may be a mixture from different species. Such antibody mutant may be a chimeric antibody and/or a humanized antibody. In general, both “chimeric antibodies” and “humanized antibodies” refer to antibodies that combine regions from more than one species. For example, “chimeric antibodies” traditionally comprise variable region(s) from a non-human animal, generally the mouse (or rat, in some cases) and the constant region(s) from a human. For the most part, humanized antibodies are chimeric antibodies that contain minimal sequence derived from non-human immunoglobulin. Generally, in a humanized antibody, the entire antibody, except the CDRs, is encoded by a polynucleotide of human origin or is identical to a human antibody except within its CDRs. The CDRs, some or all of which are encoded by nucleic acids originating in a non-human organism, are grafted into the beta-sheet framework of a human antibody variable region to create an antibody, the specificity of which is determined by the engrafted CDRs. The method for preparing such antibodies are well-known and are described in, e.g., WO 92/11018; Jones, 1986, Nature 321:522-525; Verhoeyen et al., 1988, Science 239:1534-1536, Tsurushita & Vasquez, 2004, Humanization of Monoclonal Antibodies, Molecular Biology of B Cells, 533-545, Elsevier Science (USA)).

As used herein, “fully human antibody” or “complete human antibody” refers to an antibody entirely comprising sequences originating from human genes. In some cases this may be human antibodies that have the gene sequence of an antibody derived from a human chromosome with the modifications outlined herein. Alternatively, the components of the antibody may be human but not be derived from a single gene. Thus, for example, human CDRs from one antibody can be combined with sequences, such as scaffold sequences, from one or more human antibodies. For example, a variety of germline sequences can be combined to form a human antibody or human scaffold.

Full-length antibodies comprising covalent modifications are also included within the scope of this invention. Such modifications include, but are not limited to, glycosylations, labeling and conjugation.

Labeling refers to the coupling of a detectable label with the full-length antibody. As use herein, a label includes, without being limited to: a) isotopic labels, which may be radioactive or heavy isotopes; b) magnetic labels (e.g., magnetic particles); c) redox active moieties; d) optical dyes such as chromophores, phosphors and fluorophores; enzymatic groups (e.g. horseradish peroxidase, β-galactosidase, luciferase, alkaline phosphatase); e) biotinylated groups; and f) predetermined polypeptide epitopes recognized by a secondary reporter (e.g., leucine zipper pair sequences, binding sites for secondary antibodies, metal binding domains, epitope tags, etc.).

Conjugation refers to the coupling of the full-length antibody with a polypeptide or a non-peptide molecule such as a target-binding region of a receptor, an adhesion molecule, a ligand, an enzyme, a cytokine, a chemokine, a drug, a cytotoxic agent (e.g., chemotherapeutic agents) or a toxin.

In certain embodiments, an IgG1 Fc-containing molecule is selected from the group consisting of chimeric immunoglobulins, humanized immunoglobulins, fully-human immunoglobulins, immunoglobulins being preferably selected among IgGs and optionally conjugated or labelled.

The properties of the mutant IgG1 Fc-containing molecule can be generally deduced from those of the wild-type IgG1 Fc-containing molecule except in terms of binding to C1q and Fcγ receptors since the binding of the mutant to C1q and FcγRs are controlled by the amino acid modifications at position 265, 297, and 329. Apart from these highly relevant differences, there are some minor differences in properties of the Fc-containing molecules of the invention and their corresponding wild-types, for instance a slight drop in thermostability due to a lack of N-linked glycosylation.

A further object of the invention is an isolated nucleic acid encoding a mutant IgG1 Fc-containing molecule as defined hereabove. The invention also relates to a vector comprising a nucleic acid encoding the mutant IgG1 Fc-containing molecule and to a host cell comprising the said vector. In a preferred embodiment, the nucleic acid encoding the said vector has been stably integrated in the genome of the host cell. The invention also relates to a non-human transgenic animal comprising the said nucleic acid or the said vector stably integrated within its genome.

### Uses of the Method and the Mutants According to the Invention

The Applicant showed that the substitution of amino acids 265, 297, and 329 of the IgG1 Fc region drastically impairs the affinity of the Fc mutant for C1q and for FcγRs such as FcγRI, FcγRIIa, FcγRIIb and FcγRIIa. The decrease in the affinity for these effector molecules is so pronounced that in some cases, the binding of the Fc mutant to C1q and/or to certain FcγRs cannot be observed in vitro by conventional AlphaScreen™/SPR assays. The binding of the IgG1 Fc region to C1q is essential for the induction of CDC in vivo. In the same way, the binding of the IgG1 Fc region to FcγRIIa and FcγRIIIa is a key step for the induction of ADCC and ADCP in vivo. Binding to FcγR can induce clustering of the cognate receptor, which may provide an agonistic signal through that receptor to the target cell.

Consequently, due to their poor affinity for C1q, the mutant IgG1 Fc-containing molecules of the invention are anticipated to have no CDC activity or to induce a significantly lower CDC response in vivo as compared to their wild-type counterparts (i.e., IgG1 Fc-containing molecules comprising an IgG1 Fc region with amino acids D at position 265, N at position 297, and P at position 329, wherein numbering is with reference to the EU index as in Kabat). In the same way, due to their poor affinity for certain FcγRs (in particular FcγRIIa and FcγRIIIa), the mutants of the invention are anticipated to have no ADCC activity or to induce a significantly lower ADCC response in vivo as compared to their wild-type counterparts. In the same way, the mutants of the invention are anticipated to not induce receptor clustering or agonism via FcγR engagement in vivo. The same result is also expected for in vitro CDC assays, ADCC assays and receptor clustering assays.

Due to their effector activity profiles, the mutants of the invention may find use in a wide range of scientific fields. In particular, the mutants of the invention may be used as research reagents, diagnostic agents or therapeutics.

For example, the mutants may be labeled with a fluorophore or with an isotope such as indium-111 or technetium-99m and be used for in vivo imaging since in such an application, the activation of ADCC or CDC is not required.

When used as therapeutics, the mutant may be used to convey a therapeutic agent such as radionuclides, toxins, cytokines or enzymes to a target cell for example a cancerous cell. In this case, the mutant may be a conjugate between an antibody and the cytotoxic agent and its therapeutic activity relies on the cytotoxic agent (e.g. Gilliland et al., PNAS, 1980, 77, 4539-4543).

The IgG1 Fc-containing molecule of the invention may also function as a blocking or neutralizing agent of a target molecule. It may also agonize, antagonize or inhibit a target molecule.

The IgG1 Fc-containing molecule of the invention may be used to target receptors without inducing receptor clustering or agonism via FcγR.

The target molecule may be of any kind and includes both exogenous and endogenous molecules. Target molecules (also called antigens when the polypeptide mutant is or comprises an antibody) include without being limited, viral, bacterial and fungal proteins, prions, toxins, enzymes, membrane receptors, drugs and soluble proteins.

Membrane receptors include, without being limited to, RhD antigen, CD3, CD4, CD19, CD20, CD22, CD25, CD28, CD32B, CD33, CD38, CD40, CD44, CD52, CD71 (transferrin receptor), CD80, CD86, CTLA-4, CD147, CD160, CD224, growth factor receptors like those belonging to the ErbB family of receptors ErbB1, ErbB2, ErbB3, ErbB4 (EGFR, HER2/neu, HER3, HER4), PD1, VEGF-R1, VEGF-R2, IGF-R1, PIGF-R, MHC class I and MHC class II molecules, e.g. HLA-DR, type I interferon receptor, interleukin receptors like IL-1R, IL-2R alpha, IL-2R beta and IL-2R gamma, IL-6R, hormone receptors like Millerian inhibitory substance type II receptor, LDL receptor, NKp44L, chemokine receptors like CXCR4, CCR5, TNFR, CD137, integrins, adhesion molecules like CD2, ICAM, EpCAM, G-protein-coupled receptor, etc.

Other potential target proteins include tumour markers like GD2, GD3, CA125, MUC-1, MUC-16, carcinoembrionic antigen (CEA), Tn, glycoprotein 72, PSMA, HMW-MAA other proteins such as BDCA-2 specific for DC cells, glucagon-like peptides (e.g., GLP-1, etc.), enzymes (e.g., glucocerebrosidase, iduronate-2-sulfatase, alphagalactosidase-A, agalsidase alpha and beta, alpha-L-iduronidase, butyrylcholinesterase, chitinase, glutamate decarboxylase, imiglucerase, lipase, uricase, platelet-activating factor acetylhydrolase, neutral endopeptidase, myeloperoxidase, etc.), interleukin and cytokine binding proteins (e.g., IL-18 bp, TNF-binding protein, etc.), macrophage activating factor, macrophage peptide, B cell factor, T cell factor, protein A, allergy inhibitor, cell necrosis glycoproteins, immunotoxin, lymphotoxin, tumor necrosis factor, tumor suppressors, etc.

Soluble proteins include, without being limited to, cytokines such as for instance IL-1 beta, IL-2, IL-6, IL-12, IL-23, TGF beta, TNF alpha, IFN gamma, chemokines, growth factors like VEGF, G-CSF, GM-CSF, EGF, PIGF, PDGF, IGF, hormones and inhibitory antibody such as a FVIII inhibitory, metastasis growth factor, alpha-1 antitrypsin, albumin, alpha-lactalbumin, apolipoprotein-E, erythropoietin, highly glycosylated erythropoietin, angiopoietins, hemoglobin, thrombin, anti-thrombin III, thrombin receptor activating peptide, thrombomodulin, factor VII, factor VIIa, factor VIII, factor IX, factor XIII, plasminogen activating factor, fibrin-binding peptide, urokinase, streptokinase, hirudin, protein C, C-reactive protein, B cell activating factor receptor, receptor antagonists (e.g., IL1-Ra), complement proteins, C1, C2, C3, C4, C5, C6, C7, C8, C9, factor H, factor I, factor P, other proteins such as CSAP, CD137-ligand, lectins, sialylated proteins.

In some exemplary and non-limiting embodiments, the IgG1 Fc-containing molecule may be selected from anti-CD3, anti-HER2, and anti-PD1 antibodies or molecules comprising such antibodies.

In certain embodiments, the IgG1 Fc-containing molecule comprises an anti-CD3 antibody. In certain embodiments, the molecule of the invention comprises an antibody that binds to CD3, as well as another binding moiety such as a Fynomer binding to another target, i.e. it has bispecific binding activities. Such molecules can be agonistic mAbs used for treating cancer, and are for instance described in more detail in the examples herein.

In some embodiments, the mutant IgG1 Fc-containing molecule is or comprises a neutralizing antibody directed to a target molecule selected from the group of membrane receptors, human soluble proteins, toxins, viral, bacterial and fungal proteins.

Because of its low binding to C1q and some FcγRs, the mutant of the invention is particularly appropriate to be used for the treatment of conditions in which the recruitment of the immune system through ADCC or CDC, or where clustering of the cognate receptor or agonism via FcγR, is not crucial for the therapeutic efficiency.

In some cases, the administration of the mutant IgG1 Fc-containing molecule of the invention is anticipated to induce less side-effect and less IgG-mediated cytotoxicity than most of the antibodies and immunoadhesins which do not comprise mutations at amino acid position 265, 297, and 329 in their IgG1 Fc region.

A further object of the invention is thus the use of the mutant IgG1 Fc-containing molecule of the invention for preventing or treating a pathological condition wherein FcR-mediated effects including the induction of ADCC and/or CDC responses, or the clustering of the cognate receptor via FcγR, is not desirable.

The induction of ADCC and CDC responses is not desirable when the therapeutic efficacy of the mutant does not require effector-cell activation or CDC activation. Such a mutant includes for example blocking or neutralizing antibodies.

Pathological conditions which treatment or prevention do not require the induction of CDC and ADCC, include without being limited to, graft rejection, autoimmune diseases, inflammatory diseases.

The induction of receptor clustering via FcγR is not desirable when the therapeutic efficacy of the mutant does not require FcγR mediated receptor clustering for therapeutic efficacy. Such mutants include for instance CD3/tumor antigen bispecific molecules, which require clustering of the CD3 receptor in a strictly tumor antigen dependent manner, but not in an FcγR-dependent manner.

In certain embodiments, the invention provides a FynomAb according to the invention (i.e. comprising an IgG1 Fc-region with a CH2 domain wherein the amino acid at position 265 is not D, the amino acid at position 297 is not N, and the amino acid at position 329 is not P, wherein numbering is according to the EU index as in Kabat) having an antibody part binding to CD3 and a Fynomer part binding to CD33.

Another object of the invention is the use of a mutant of the invention for preparing a pharmaceutical composition.

A further object of the invention is to provide pharmaceutical compositions comprising the said mutant. When the mutant IgG1 Fc-containing molecule is an antibody, the mutant may be present in the form of monoclonal or polyclonal antibodies. The pharmaceutical compositions are prepared by mixing the polypeptide mutant having the desired degree of purity with optional physiologically acceptable carrier, excipients or stabilizers in the form of lyophilised formulations or aqueous solutions.

The pharmaceutical composition of the invention may be formulated according to standard methods such as those described in Remington: The Science and Practice of Pharmacy (Lippincott Williams & Wilkins; Twenty first Edition, 2005).

Pharmaceutically acceptable excipients that may be used are, in particular, described in the Handbook of Pharmaceuticals Excipients, American Pharmaceutical Association (Pharmaceutical Press; 6th revised edition, 2009).

In order to treat a patient in need, a therapeutically effective dose of the mutant IgG1 Fc-containing molecule of the invention may be administered. By “therapeutically effective dose” herein is meant a dose that produces the effects for which it is administered. The exact dose will depend on the purpose of the treatment, and will be ascertainable by one skilled in the art using known techniques. Dosages may range from 0.0001 to 100 mg/kg of body weight or greater, for example 0.001, 0.01, 0.1, 1.0, 10, or 50 mg/kg of body weight, with 0.001 to 10 mg/kg being preferred. As is known in the art, adjustments for protein degradation, systemic versus localized delivery, and rate of new protease synthesis, as well as the age, body weight, general health, sex, diet, time of administration, drug interaction and the severity of the condition may be necessary, and will be ascertainable with routine experimentation by those skilled in the art.

Administration of the pharmaceutical composition comprising a mutant IgG1 Fc-containing molecule of the invention may be done in a variety of ways, including, but not limited to, orally, subcutaneously, intravenously, parenterally, intranasally, intraortically, intraocularly, rectally, vaginally, transdermally, topically (e.g., gels), intraperitoneally, intramuscularly, intrapulmonary.

The mutant IgG1 Fc-containing molecules described herein may optionally be administered with other therapeutics concomitantly, i.e., the therapeutics described herein may optionally be co-administered with other therapies or therapeutics, including for example, small molecules, other biologicals, radiation therapy, surgery, etc.

### Exemplary Embodiments of the Described Subject Matter

To better and more fully describe the subject matter herein, this section provides enumerated exemplary embodiments of the subject matter presented.

Enumerated embodiments:

### EMBODIMENTS

1. A recombinant IgG Fc-containing molecule, comprising a CH2 domain in which the amino acid at position 265 is different from aspartic acid (D), the amino acid at position 297 is different from asparagine (N), and the amino acid at position 329 is different from proline (P), wherein the numbering is indicated by the EU index as in Kabat.
2. The molecule of embodiment 1, wherein the molecule has reduced binding to C1q and to at least one Fcγ receptor (FcγR), as compared to an IgG1 Fc-containing molecule having a wild-type CH2 domain that comprises D at position 265, N at position 297 and P at position 329.
3. The molecule of embodiment 1 or 2, wherein the molecule retains binding to FcRn.
4. The molecule of any one of embodiments 2-3, wherein at least one FcγR is FcγRI, FcγRIIa, FcγRIIb, FcγRIIIa, and FcγRIIIb.
5. The molecule of any one of embodiments 1-4 wherein
   i. the amino acid at position 265 is alanine (A), asparagine (N) or glutamic acid (E),
   ii. the amino acid at position 297 is alanine (A), aspartic acid (D), or glutamine (Q), and
   iii. the amino acid at position 329 is replaced with alanine (A), glycine (G), or serine (S).
6. The molecule of any one of embodiments 1-5, wherein the Fc domain amino acid sequence is at least 90% identical to the amino acid sequence of the human IgG1 Fc domain (SEQ ID NO: 43).
7. The molecule of any one of embodiments 1-6, wherein the molecule is an antibody, an Fc region, an Fc-fusion protein, or antibody fusion protein such as a FynomAb.
8. The molecule according to any one of embodiments 1-7, wherein the molecule is an antibody.
9. The molecule according to any one of embodiments 1-7, wherein the molecule is an antibody fusion protein.
10. The molecule according to any one of embodiments 1-7, wherein the molecule is a FynomAb.
11. The molecule of any one of embodiments 1-10, wherein the molecule comprises an Fc region comprising a sequence according to any one of SEQ ID NOs: 43, 52, 53, 54, 55, 56, 57, or 58, wherein amino acids D at position 265, N at position 297 and P at position 329 are replaced by other amino acids.
12. A recombinant polynucleotide encoding the molecule of any one of the preceding embodiments.
13. A vector comprising the polynucleotide of embodiment 12.
14. A host cell comprising the recombinant polynucleotide of embodiment 12 or the vector of embodiment 13.
15. A method of making a recombinant IgG1 Fc-containing molecule, comprising a CH2 domain in which amino acids at position 265, 297, and 329 indicated by the EU index as in Kabat are replaced by other amino acids, the method comprising the steps of:
   a. providing a nucleic acid encoding a wild-type IgG1 Fc-containing molecule,
   b. modifying the nucleic acid provided in step (a) so as to obtain a nucleic acid encoding a recombinant IgG1 Fc-containing molecule wherein the amino acids at position 265, 297, and 329 are replaced with amino acids other than D, N and P, respectively, and
   c. expressing the nucleic acid obtaining in step (b) in a host cell and recovering the said mutant.
16. A recombinant polypeptide comprising
   a. at least one binding domain capable of binding a target molecule; and
   b. an IgG1 Fc domain wherein the amino acids at positions 265, 297, and 329 according to the EU index as in Kabat are different from D, N, and P, respectively,
   wherein the polypeptide is capable of binding the target molecule without triggering significant lymphocyte activation, complement dependent lysis, and/or cell mediated destruction of the target molecule and/or cell that displays the target molecule on its surface.
17. The recombinant polypeptide of embodiment 16, wherein the at least one binding domain is selected from the group consisting of a binding site of an antibody, a Fynomer, an enzyme, a hormone, an extracellular domain of a receptor, a cytokine, an immune cell surface antigen, a ligand, and an adhesion molecule.
18. The recombinant polypeptide of embodiment 16 or 17, wherein the Fc domain is at least 90% identical to the amino acid sequence of the human IgG1 Fc domain (SEQ ID NO: 43).
19. The recombinant polypeptide of any one of embodiments 16-18 wherein the binding domain is the binding site of an antibody.
20. A pharmaceutical composition comprising the IgG1 Fc-containing molecule of any one of embodiments 1-11, the recombinant polynucleotide of embodiment 12, the vector of embodiment 13, or the recombinant polypeptide of any one of embodiments 16-19, and a pharmaceutically acceptable excipient.
21. A method of treating disease or disorder, comprising administering to a subject or patient the IgG1 Fc-containing molecule of any one of embodiments 1-11, the recombinant polynucleotide of embodiment 12, the vector of embodiment 13, the recombinant polypeptide of any one of embodiments 16-19, or the pharmaceutical composition according to embodiment 20.
22. The method of embodiment 21, wherein the disease or disorder is cancer.
23. The IgG1 Fc-containing molecule of any one of embodiments 1-11, the recombinant polynucleotide of embodiment 12, the vector of embodiment 13, the recombinant polypeptide of any one of embodiments 16-19, or the pharmaceutical composition according to embodiment 20, for use in treating a disease or disorder.
24. The IgG1 Fc-containing molecule, recombinant polynucleotide, vector, or recombinant polypeptide of embodiment 23, wherein the disease or disorder is cancer.
25. Use of the IgG1 Fc-containing molecule of any one of embodiments 1-11, the recombinant polynucleotide of embodiment 12, the vector of embodiment 13, the recombinant polypeptide of any one of embodiments 16-19, or the pharmaceutical composition according to embodiment 20, for the manufacture of a medicament for treating a disease or disorder.
26. Use according to embodiment 25, wherein the disease or disorder is cancer.
27. A method for producing a recombinant IgG1 Fc-containing molecule, the method comprising expressing the recombinant polynucleotide of embodiment 12 in a host cell and harvesting the the recombinant polypeptide.

### EXAMPLES

The following examples are provided to supplement the prior disclosure and to provide a better understanding of the subject matter described herein. These examples should not be considered to limit the described subject matter. It is understood that the examples and embodiments described herein are for illustrative purposes only and that various modifications or changes in light thereof will be apparent to persons skilled in the art and are to be included within, and can be made without departing from, the true scope of the invention.

### Example 1. Expression and Purification of Fc Mutated Antibodies

Several antibodies based on mAb1, a human IgG1 antibody specific to human CD3, were produced with different mutations in the CH2 domain. The mutations were:
i) N297A,
ii) D265 plus P329A (DAPA),
iii) D265 plus N297A plus P329A (DANAPA), and
iv) L234A plus L235A (LALA)
   (EU numbering according to Kabat (Kabat, E. A. (1991). Sequences of proteins of immunological interest, Bethesda, Md.: U.S. Dept. of Health and Human Services, Public Health Service, National Institutes of Health, 1991).

For expression of antibodies, a leader sequence is typically present, which is cleaved off and no longer present in the secreted product. An example of a leader sequence used for expression in the examples described herein is provided in SEQ ID NO: 42, and an example of a nucleotide sequence encoding such is provided in SEQ ID NO: 41.

Expression vectors encoding the antibodies with the different Fc mutations were transiently transfected into FreeStyle CHO-S cells and expressed in serum-free/animal component-free media for 6 days. The anti-CD3 antibodies were purified from the supernatants by Protein A affinity chromatography (GE-Healthcare cat no 89928) with an AKTA Purifier instrument (GE Healthcare) and dialyzed against PBS. Concentrations were determined by absorbance measurement at 280 nm.

SEC was performed using a SEC-5 column (Agilent, 5 μm particle size, 300A) on an Agilent HPLC 1260 system. 10 μl purified protein was loaded on the column and elution was recorded by OD280 measurement.

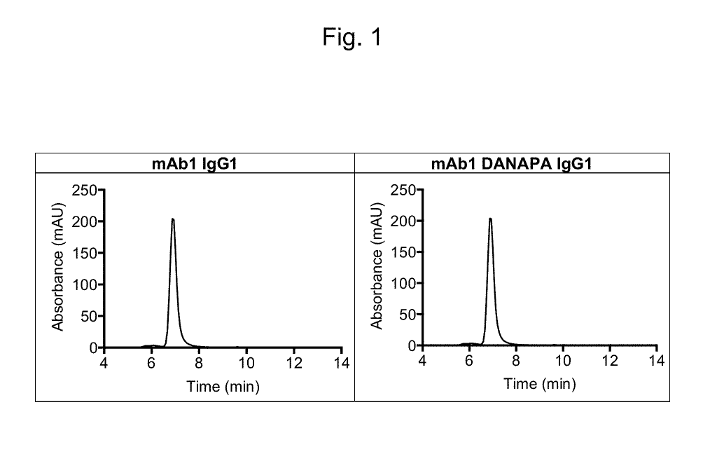

The Fc mutated antibody mutants could be purified with good yields and high purity by single-step protein A affinity chromatography. Yields are listed in Table 1. As found by SEC, all proteins were approximately 95% monomeric. The SEC profiles of mAb1 IgG and mAb1 DANAPA IgG are shown in FIG. 1.

These results demonstrate the DANAPA triple mutation inserted into a human IgG1 sequence retains good expression and monodispersity, which both are key criteria for a pharmaceutical product.

TABLE 1
Protein Yields of mAb1 mutants

| | | Heavy | Light | |
|---|---|---|---|---|
| | | Chain | Chain | Purification |
| | | SEQ | SEQ | yield |
| Clone ID | Fc mutations | ID NO: | ID NO: | (mg/l) |
| mAb1 IgG1 | none (wild-type) | 2 | 4 | 98 |
| mAb1 N297A | N297A | 49 | 4 | 97 |
| IgG1 | | | | |
| mAb1 DAPA | D265A, P329A | 47 | 4 | 66 |
| IgG1 | | | | |
| mAb1 | D265A, N297A, | 45 | 4 | 62-132 |
| DANAPA IgG1 | P329A | | | |
| mAb1 LALA | L234A, L235A | 51 | 4 | 88 |
| IgG1 | | | | |

### Example 2: Fc Mutated Antibodies Bind to CD3-Expressing Cells with Identical Affinity as the Unmodified Antibody

Different Fc mutated mAb1 were titrated on CD3⁺ Jurkat cells (ATCC TIB-152™) to assess the binding affinity to human CD3. Serial dilutions of Fc mutated antibodies between 50 nM and 0.13 pM concentration were added to Jurkat cells, and bound antibody was detected with an anti-human IgG-Alexa488 conjugated antibody. The mean fluorescent intensity (MFI) determined on a cytometer was plotted against the antibody concentration on a logarithmic scale.

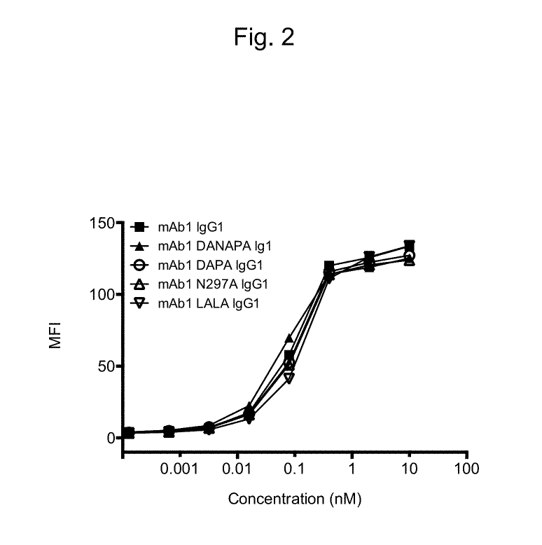

The binding curves obtained on CD3⁺ Jurkat cells are shown in FIG. 2.

The Fc mutated antibody mutants bound to CD3 with identical affinity, indicating that the Fc mutations do not have any impact on target cell binding.

### Example 3: MAb1 DANAPA IgG1 does not Induce Lymphocyte Activation

In order to investigate the effect of Fc mutated mAb1 on immune cell activation, freshly isolated human PBMC were incubated in the presence of Fc mutated mAb1. Immune cell activation was detected by i) CD69 surface staining after 14 h incubation, or by ii) quantification of IFNγ in the supernatant after 3 days incubation. Human PBMC were isolated from buffy coat preparations collected by Blutspende Bern, Switzerland, one day before PBMC isolation. PBMC were isolated by density centrifugation, using Pancoll tubes (Pan-BioTech) according to the manufacturer's instructions. After PBMC isolation, residual red blood cells were lysed with 1×RBC lysis buffer (Miltenyi).

100′000 freshly isolated PBMC were mixed with various Fc mutants of mAb1 at serial dilutions (concentrations between 300 nM and 0.15 pM) in a total volume of 200 μl RPMI1640 supplemented with 10% heat-inactivated FBS in the wells of a 96-well U-bottom plate. As positive control, PBMC were incubated in the presence of anti-CD2/CD3/CD28 activation MACSibeads contained in the human T cell activation/expansion kit purchased from Miltenyi.

CD69 surface expression was determined after 14 h incubation. The contents of the assay wells were mixed, and 100 μl of each well was transferred into a 96-well U-bottom plate for subsequent CD69 staining. Cells were pelleted and resuspended in 40 μl anti-CD69-FITC conjugated antibody (BD Biosciences) in FACS buffer containing 1% FBS and 0.2% sodium azide. After 45 min incubation on ice, unbound antibody was washed off, samples were fixed in 50 μl 1.8% formalin for 15 min on ice, and analyzed on a Guava easeCyte 8HT flow cytometer (Millipore). The percentage CD69 positive lymphocytes were plotted against the antibody concentration on a logarithmic scale.

IFNγ levels in the supernatant were determined by sandwich ELISA after 3 days incubation, using the BD OptEIA human IFNγ ELISA set (BD biosciences) according to the manufacturer's instructions. IFNγ concentrations were plotted against the antibody concentration on a logarithmic scale.

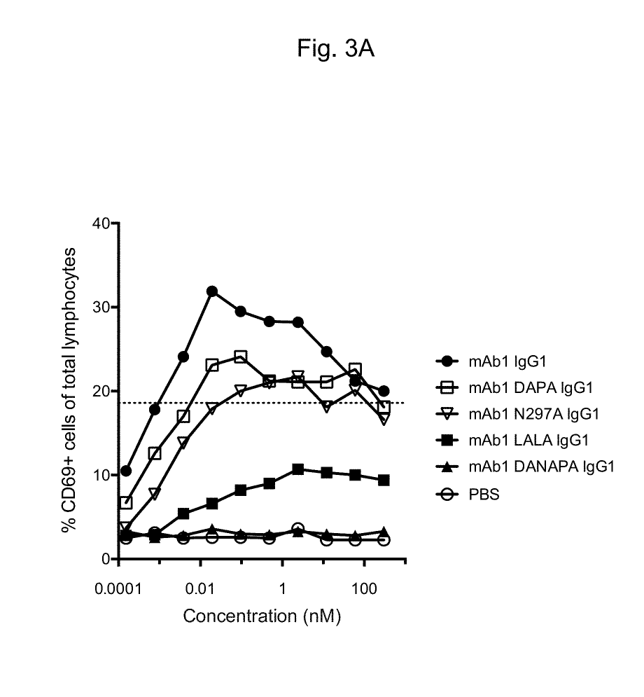

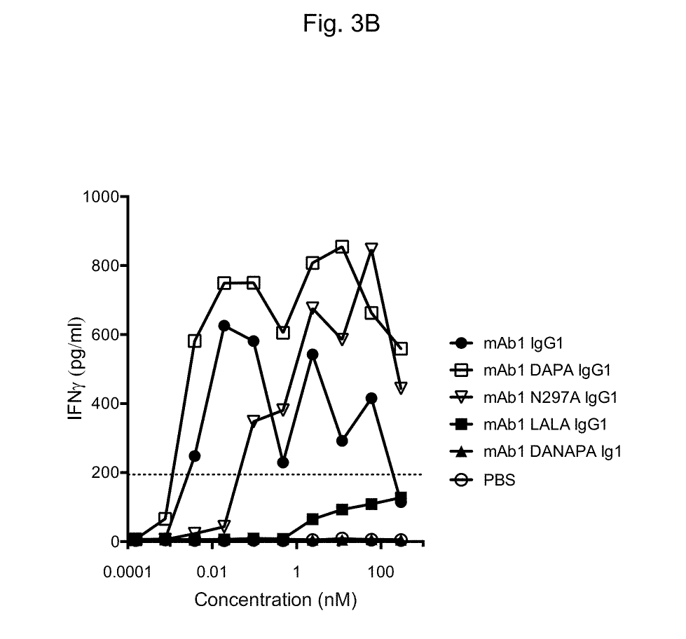

Unexpectedly, mAb1 DANAPA IgG1 was the only construct that did not induce lymphocyte activation, as demonstrated by the lack of induction of CD69 expression on PBMC (FIG. 3A), and of IFNγ in the culture supernatant (FIG. 3B). In contrast, all other mutants which contain single or combined Fc mutations previously reported to reduce FcR binding, still induced significant lymphocyte activation. Importantly, the DANAPA Fc sequence led to better silencing than the N297A Fc sequence or the LALA Fc sequence, both silenced Fc sequences used in several clinical-stage therapeutic Fc containing proteins for which minimal FcR interaction is desired. These results suggest that the DANAPA Fc sequence confers a strongly reduced potential to induce T cell activation and cytokine release in human PBMC assays.

### Example 4: DANAPA IgG1 Shows Minimal Binding to Human Fcγ Receptors

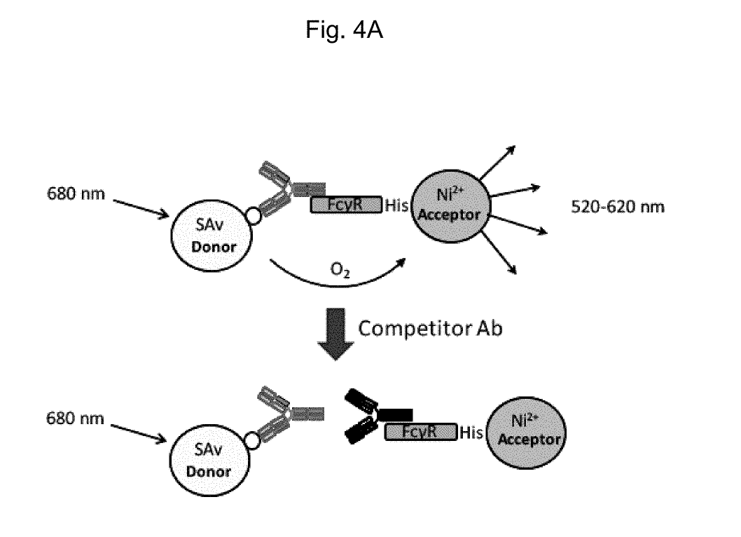

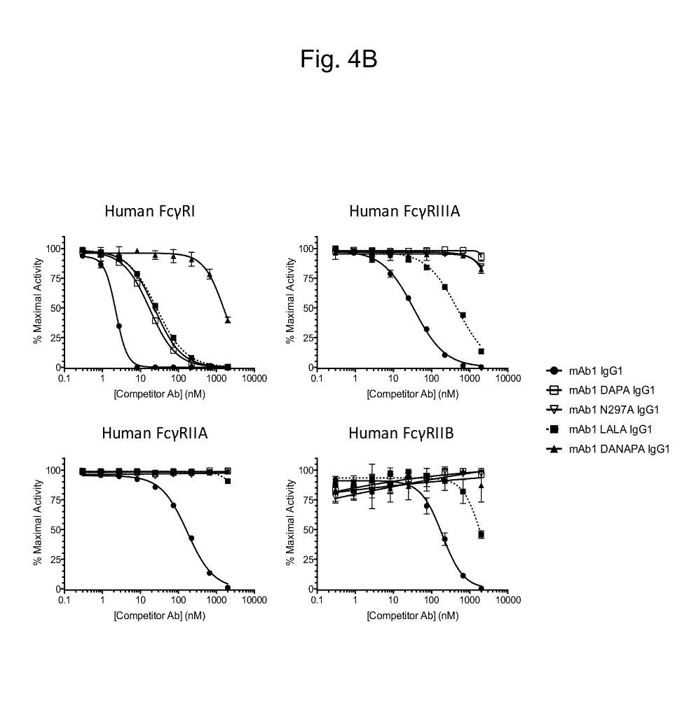

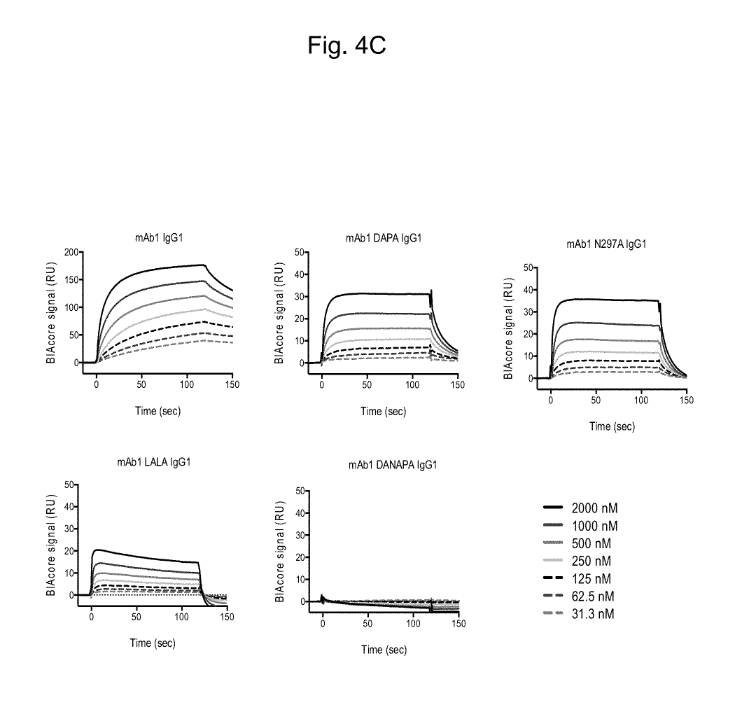

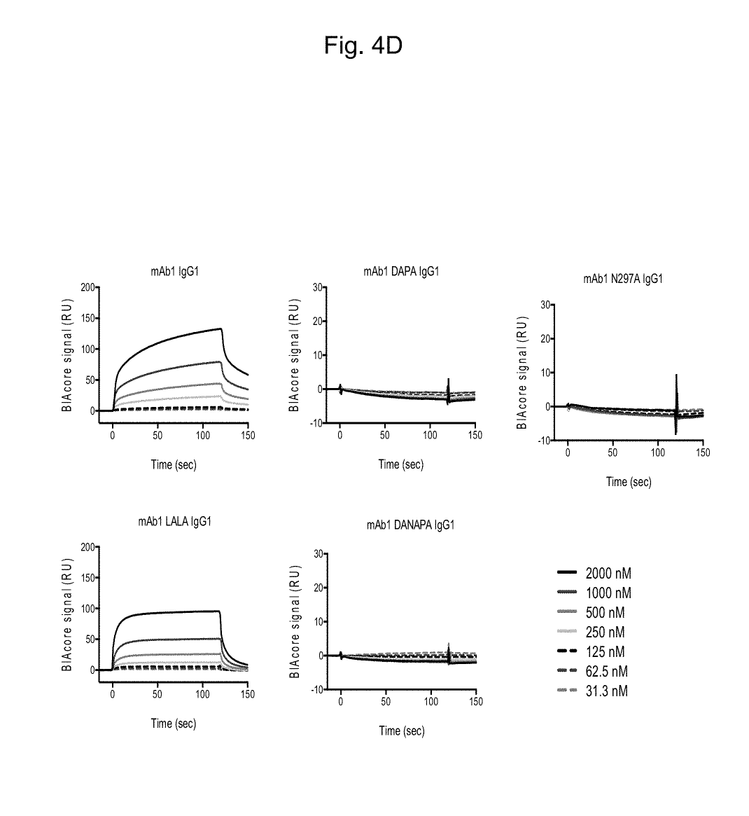

Binding to FcγRI (CD64), FcγRIIA (CD32A), FcγRIIB (CD32B) and FcγRIIIA (CD16A) was characterized by AlphaScreen™ competition assay (Vafa, O., G. L. Gilliland, R. J. Brezski, B. Strake, T. Wilkinson, E. R. Lacy, B. Scallon, A. Teplyakov, T. J. Malia and W. R. Strohl (2014). Methods 65(1): 114-126). The assay is schematically illustrated in FIG. 4A. A biotinylated control antibody is captured on Streptavidin Donor beads; His-tagged Fcγ receptors are captured on Ni²⁺ Acceptor beads; serial dilutions of unlabelled antibodies with Fc of interest are applied as competitors. This format produces a reduction in the signal when receptor binding of the competitors takes place.

B21M, a human IgG1 control antibody specific to respiratory syncytial virus and believed not to bind specifically to any targets in healthy mammals (Vafa, O., G. L. Gilliland, R. J. Brezski, B. Strake, T. Wilkinson, E. R. Lacy, B. Scallon, A. Teplyakov, T. J. Malia and W. R. Strohl (2014). Methods 65(1): 114-126), was labeled with biotin (SureLINK Chromophoric Biotin Labeling Kit, KPL). 0.2 μg/ml biotinylated B21M IgG1 control antibody, Fc mutated test antibodies (400 μg/ml, and eight serial 3-fold dilutions thereof), His-tagged human Fcγ receptors (R&D, carrier-free formulation), Ni²⁺-acceptor beads (Perkin Elmer, 1:250 diluted), and Streptavidin donor beads (Perkin Elmer, 1:250 diluted) were mixed in assay buffer (PBS, 0.05% BSA, 0.01% Tween 20, pH 7.2) in the order indicated above. The human Fcγ receptors were used at the following concentrations: FcγRI and FcγRIIIA at 200 ng/ml; FcγRIIA at 10 ng/ml; FcγRIIB at 14 ng/ml.

For the binding assessment on FcγRI, biotinylated B21M LALA IgG1 was used instead of B21M IgG1 (heavy chain SEQ ID NO: 18; light chain SEQ ID NO: 32) in order to increase the sensitivity of the assay. B21M LALA IgG1 (heavy chain SEQ ID NO: 30; light chain SEQ ID NO: 32) carries two Alanine substitutions at L234 and L235 (see also Example 1) which reduce the binding affinity to FcγRI.

After 30 min incubation, the plates were analyzed in an EnVision plate reader. % Max signal was obtained from raw EnVision data by normalization to the minimal and maximal signal, using the following equation:

% Max=(Exp−Min)/(Max−Min)*100

where
Exp=EnVision raw well signal
Min=Minimum signal obtained at highest competitor concentration across all tested competitors on a plate.
Max=Maximum signal, i.e. typically in the absence of competitor.

The % Max values were plotted in GraphPad Prism as mean±standard deviation (n=3) on the y-axis, and log (inhibitor) on the x-axis. Data was fitted by non-linear regression, using a four parameter Log (inhibitor) vs. response model with variable Hill slope,

To confirm the results of the AlphaScreen™ competition assay, binding of Fc mutated mAb1 mutants to the high-affinity FcγRI (CD64) and to the low-affinity FcγRIIIA (CD16A) was analyzed by surface plasmon resonance (SPR). A BIAcore CM5 chip was coated with 1400 RU of human recombinant FcγRIIIA (158F; R&D Systems) or with 1500 RU of human recombinant FcγRI (Sino Biological) using the BIAcore amine coupling kit (GE healthcare). Serial two-fold dilutions of Fc mutated mAb1 at concentrations between 2000 nM and 31 nM were prepared and injected at 30 μl/min in PBS pH 7.4 supplemented with 0.05% Tween-20 over the FcR coated chip surfaces and over an uncoated reference surface. The chip surface was regenerated with 10 mM NaOH between injections. The obtained binding curves were reference substracted, then buffer substracted, and the resulting double-referenced curves were evaluated using the BIAcore evaluation software, using either a 1:1 Langmuir kinetic model to obtain kinetic association and dissociation constants, kₒₙ and k_{off}, from which the thermodynamic dissociation constant K_{D} was calculated as k_{off} I/kₒₙ, or using a steady-state affinity model to directly obtain the thermodynamic dissociation constant, K_{D}.

The results of the AlphaScreen™ competition assay are shown in FIG. 4B and in Table 2. mAb1 DANAPA IgG1 showed minimal competition on FcγRI (IC₅₀>1000 nM) that is more than 400-fold reduced as compared to unmodified IgG, indicating that this Fc sequence has minimal residual FcγRI binding activity. Unexpectedly, the DANAPA Fc showed strikingly reduced binding to FcγRI compared to the LALA Fc or the N297A Fc sequences used in clinical-stage antibodies for which minimal FcR interaction is desired. mAb1 LALA IgG, mAb1 N297A IgG1 and mAb1 DAPA IgG1 showed reduced but still more than 37-fold stronger binding to human FcγRI than mAb1 DANAPA IgG1 (IC₅₀=27 nM, 24 nM and 18 nM).

No binding to any other human FcγR was found for mAb1 DANAPA IgG1, mAb1 DAPA IgG1, and mAb1 N297A IgG1. mAb1 LALA IgG1 was observed to bind to FcγRIIIA and very weakly to FcγRIIB.

The results of the BIAcore binding assays are shown in FIG. 4C and Table 3 (FcγRI binding), and in FIG. 4D and Table 4 (FcγRIIIA binding). Unexpectedly, mAb1 DANAPA IgG1 shows completely abrogated binding to FcγRI. mAb1 DAPA IgG1, mAb1 N297A IgG1 and mAb1 LALA IgG1 retain residual binding activity to FcγRI, albeit with reduced affinity as compared to mAb1 IgG1. mAb1 DANAPA IgG1 shows no binding to human FcγRIIIA. Similarly, mAb1 DAPA IgG1 and mAb1 N297A IgG1 show no binding, whereas mAb1 LALA IgG1 shows residual binding to FcγRIIIA, albeit with reduced activity as compared to mAb1 IgG1.

Conclusively, these results demonstrate that the DANAPA Fc sequence has strikingly reduced binding to human FcγRI, FcγRIIA, FcγRIIB and FcγRIIIA. The degree to which the DANAPA Fc sequence has reduced FcR binding activity is far superior compared to other single or combined Fc mutations previously known to lead to reduced FcγR binding activity. These results further suggest that the main difference between DANAPA Fc and the other Fc's tested here lies in the strikingly reduced binding to FcγRI.

TABLE 2
IC₅₀ values for competition binding to FcγRI

| | | mAb1 | mAb1 | mAb1 | mAb1 |
|---|---|---|---|---|---|
| | mAb1 | DAPA | N297A | LALA | DANAPA |
| | IgG1 | IgG1 | IgG1 | IgG1 | IgG1 |
| IC₅₀ (nM) | 2.27 | 17.74 | 23.89 | 27.12 | >1000 |
| IC₅₀ relative to | 1 | 7.8 | 10.5 | 11.9 | >400 |
| IgG1 | | | | | |

TABLE 3
Parameters for binding to FcγRI (BIAcore)

| | kₒₙ | k_{off} | K_{D} | K_{D} (nM) |
|---|---|---|---|---|
| Analyte | (M⁻¹ s⁻¹) | (s⁻¹) | (nM) | steady-state |
| mAb1 IgG1 | 6.49E+04 | 7.29E−03 | 112 | 243 |
| mAb1 DANAPA | n.b. | n.b. | n.b. | n.b. |
| IgG1 | | | | |
| mAb1 N297A IgG1 | 1.52E+05 | 1.12E−01 | 737 | 1512 |
| mAb1 DAPA IgG1 | 1.03E+05 | 5.95E−02 | 575 | 1186 |
| mAb1 LALA IgG1 | n.d. | n.d. | n.d. | 1554 |

n.b.: no binding observed (data was analyzed)
n.d.: not determined (binding observed, but kinetic data was not analyzed)

TABLE 4
Parameters for binding to FcγRIIIA (BIAcore)

| | kₒₙ | k_{off} | K_{D} | K_{D} (nM) |
|---|---|---|---|---|
| Analyte | (M⁻¹ s⁻¹) | (s⁻¹) | (nM) | steady-state |
| mAb1 IgG1 | 3.18E+04 | 2.14E−02 | 674 | 956 |
| mAb1 DANAPA | n.b. | n.b. | n.b. | n.b. |
| IgG1 | | | | |
| mAb1 N297A IgG1 | n.b. | n.b. | n.b. | n.b. |
| mAb1 DAPA IgG1 | n.b. | n.b. | n.b. | n.b. |
| mAb1 LALA IgG1 | 1.42E+04 | 1.11E−01 | 7782 | 13828 |

n.b.: no binding observed (data was not analyzed)

### Example 5: DANAPA IgG1 Fc Shows Reduced FcγR Binding in the Context of Different Antibody Sequences

In addition to mAb1 DANAPA IgG1 presented in the previous examples above, three antibodies with different Fab sequences and different cognate targets were generated in DANAPA IgG1 format as described in Example 1 for mAb1: an anti-CD3 antibody mAb2 (heavy chain SEQ ID NO: 14; light chain SEQ ID NO: 16), an anti-HER2 antibody (heavy chain SEQ ID NO: 10; light chain SEQ ID NO: 12), and an anti-PD1 antibody (heavy chain SEQ ID NO: 6; light chain SEQ ID NO: 8). FcγR binding activity was compared to mAb1 DANAPA IgG1 in an AlphaScreen™ competition assay as described in Example 4.

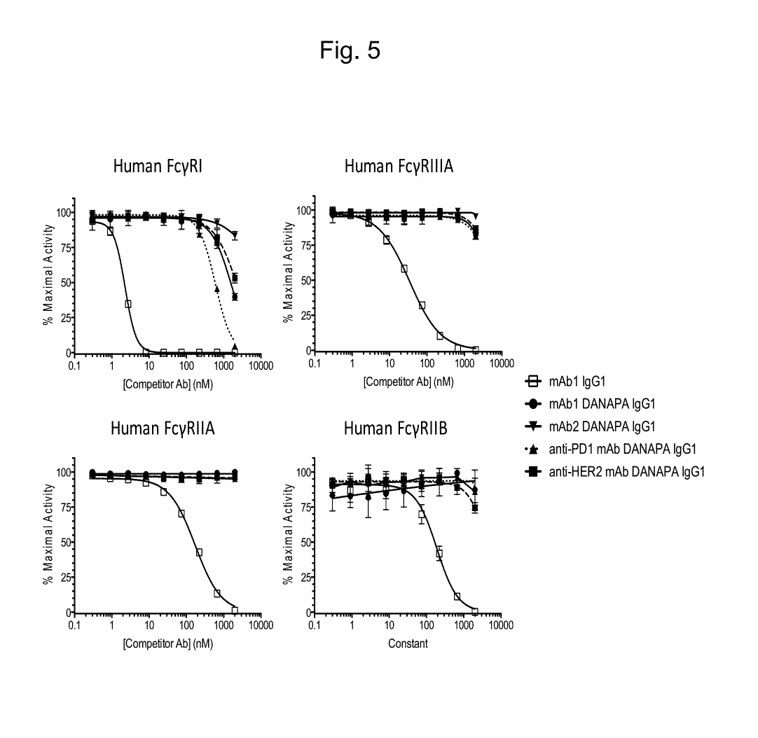

The results are shown in FIG. 5. The CD3-specific antibody mAb2, the HER2-specific antibody and the PD-1 specific antibodies in DANAPA IgG1 format all show minimal binding to FcγRI (IC₅₀=600 nM or higher), and no binding to the other tested FcR. These data show that the DANAPA IgG1 sequence strongly reduces FcR binding irrespective of Fab sequence or cognate target and potentially confers minimal FcγR binding to virtually any antibody.

### Example 6: MAb1 with Substitutions at D265, N297, and P329 Show Reduced Binding to Human FcγR1

In order to determine whether the strongly reduced ability of DANAPA Fc to bind to Fc receptors can be mirrored by substituting the same set of residues (i.e. D265, N297 and P329) with amino acids different than alanine, mAb1 with the following substitutions were generated as described in Example 1:
a) D265N, N297D, P329G (referred to as DNNDPG) (heavy chain SEQ ID NO: 18; light chain SEQ ID NO: 4)
b) D265E, N297Q, P329S (referred to as DENQPS) (heavy chain SEQ ID NO: 20; light chain SEQ ID NO: 4)
   The FcγRI binding activity was compared to mAb1 DANAPA IgG1 in an AlphaScreen™ competition assay as described in Example 4.

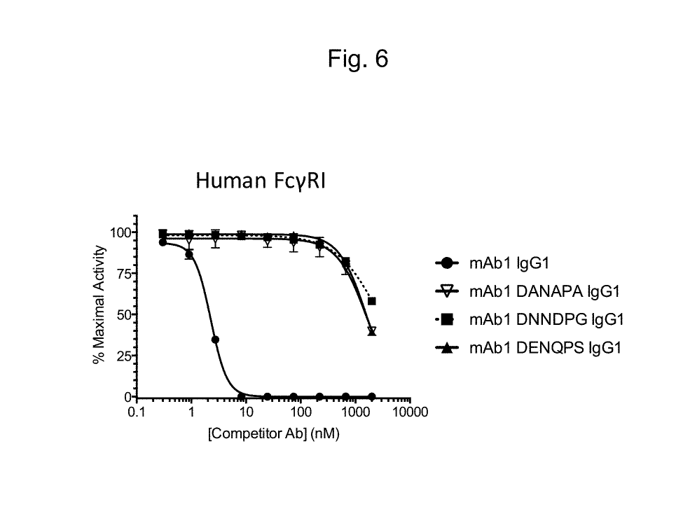

The results are shown in FIG. 6. All three antibodies show minimal binding to FcγRI, the interaction which apparently is most challenging to abrogate by Fc engineering (see Example 4). These results indicate that FcγRI interaction can be reduced by substituting the three residues, D265, N297, and P329, with different amino acid residues and not solely by substitutions with alanine.

### Example 7: DANAPA IgG1 Abrogates C1Q Binding

Binding of C1q to the antibody Fc is the initial step in the induction of antibody-mediated complement activation and subsequent complement mediated target cell lysis. mAb1 DANAPA IgG1 binding to human C1q was measured in an SPR binding assay on a BIAcore T100 instrument. Antibodies were coated onto a CM5 chip via amine coupling at coating density of 5000 RU Human C1q (EMD Millipore) was injected in running buffer (PBS pH7.4, 0.05% TWEEN-20) at 200 nM and three-fold serial dilutions thereof at a flow rate of 30 μl/min. Binding was recorded, and K_{D} was determined by curve fitting using the BIAcore software using a steady-state affinity model.

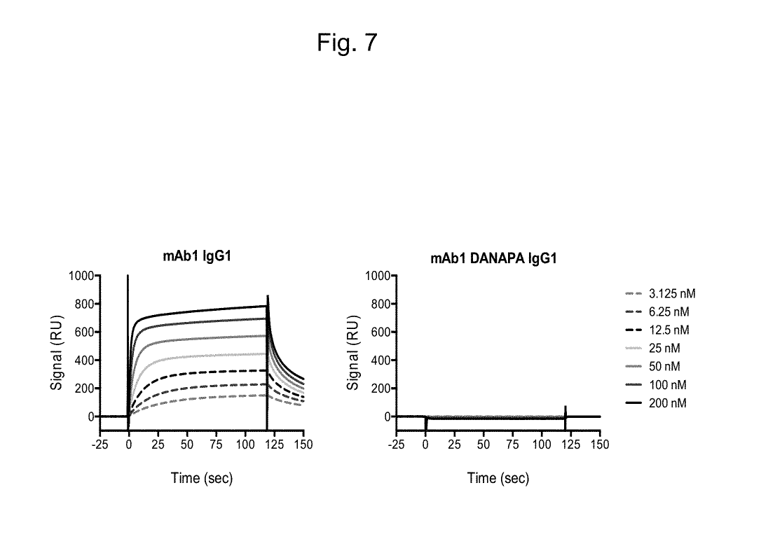

The results of the experiment are shown in FIG. 7. mAb1 IgG1 showed strong binding to C1q with an apparent K_{D} of 30 nM, which is in agreement with published affinity values for this interaction and validates the assay set-up (Moore G L, et al. (2010), Mabs 2(2): 181-189).

mAb1 DANAPA IgG1 did not show any detectable binding to C1q.

Noteworthily, a similar Fcγ 1 sequence, the DANA Fcγ1, which combines the D265A and the N297A mutations but lacks the P329A mutation present in DANAPA Fcγ11, has been described in literature to show residual C1q binding (Gong, Q, et al. (2005), J Immunol 174(2): 817-826).

In contrast to DANA Fcγ1, mAb1 DANAPA IgG1 strikingly demonstrated complete loss of binding to C1q.

### Example 8: DANAPA Mutations do not Impair Binding to Human FcRn

The interaction of IgG Fc with FcRn plays an important role in antibody turnover (Kuo T T and V G Aveson (2011), MAbs 3(5): 422-430). IgG that have been taken up by cells via pinocytosis engage with the FcRn receptor in the acidic environment of the endosomes. FcRn recycles the IgG back to the cell surface where the antibody dissociates from FcRn at neutral or basic pH and thus is rescued from lysosomal degradation. This mechanism provides an explanation for the long serum half-life of IgG. Therefore, in order to have a long circulation half-life, it is important that antibodies with substitutions in the Fc retain full binding to FcRn at acidic pH and readily dissociate at neutral pH.

While human FcγR bind to residues in the lower hinge and to the CH2 domain of IgG antibodies (Woof J M and D R Burton (2004), Nat Rev Immunol 4(2): 89-99), human FcRn interacts with several residues in the CH2-CH3 interface (Martin W L, et al. (2001), Mol Cell 7(4): 867-877). Therefore, mutations introduced with the aim to reduce FcR binding may have an impact on the Fc-FcRn interaction. For instance, Shields et al. observed that some mutations in the lower hinge or the CH2 domain that led to reduced FcγR binding also resulted in reduced FcRn binding (e.g. E233P, Q295A). Therefore, it is of particular importance to assess the impact of the DANAPA mutations on FcRn binding.

Binding of DANAPA IgG1 to FcRn was analyzed by surface plasmon resonance (SPR). A BIAcore CM5 chip was coated with 600 RU of human recombinant FcRn (Sino Biological) using the BIAcore amine coupling kit (GE healthcare). Serial two-fold dilutions of DANAPA Fc mutated mAb1 and of unmutated mAb1 IgG1 at concentrations between 2000 nM and 31 nM were prepared and injected at 30 μl/min in PBS pH 6.0 supplemented with 0.05% Tween-20 over the FcRn-coated and over an uncoated reference surface. Between injections, the chip surface was regenerated with PBS pH 7.4. The obtained binding curves were reference substracted, then buffer substracted, and the resulting double-referenced curves were evaluated using the BIAcore evaluation software, using a steady-state affinity model to obtain the thermodynamic dissociation constant, K_{D}.

The results of the binding assay to human FcRn are shown in FIG. 8. The dissociation constant, K_{D}, was 500 nM for mAb1 DANAPA IgG, and 470 nM for mAb1 IgG1, indicating that there is no difference in binding to human FcRn. mAb1 DANAPA IgG1 shows rapid dissociation at neutral pH with essentially identical dissociation kinetics as mAb1 IgG1. These results suggest that mAb1 DANAPA IgG1 retains IgG1-like binding to FcRn despite abrogated binding to FcγR.

### Example 9: MAb1 DANAPA IgG1 Exhibits IgG1-Like Pharmacokinetic Profile

Good pharmacokinetics properties, i.e. a long half-life in the circulation, is one of the key criteria that an antibody-based pharmaceutical product must meet. Engineering of antibody Fc sequences may have unexpected effects on the pharmacokinetic profile. For example, antibodies with an Fc sequence containing five mutations to reduce Fc receptor binding (“LFLEDANQPS” [note: the last P to S mutation being at position 331 according to Kabat numbering, i.e. at a position differing from the mutants in the instant disclosure]) had a 3- to 5-fold increased clearance compared to a wild-type IgG1, resulting in a shorter terminal half-life than the corresponding wild-type IgG1 (WO2014108483).

The pharmacokinetic profile of mAb1 DANAPA IgG1 in C57BL/6 mice (Charles River) was investigated and compared to mAb1 IgG1. Five C57BL/6 mice were injected i.v. with 10 mg/kg mAb1 DANAPA IgG1 or mAb1 IgG1. After 10 min, 6, 24, 48, 96, 120, 144, 168, 192 and 216 hours, blood was collected into EDTA coated microvettes (Sarstedt), centrifuged for 10 min at 9300 g and the serum levels of mAb1 DANAPA IgG1 and of mAb1 IgG1 were determined by an Fc specific sandwich ELISA. Transparent maxisorp microtiter plates (Nunc) were coated with 440-fold diluted Fc-specific anti-human IgG1 capture antibody (12134, Sigma). After blocking with 2% BSA (Sigma) in PBS, 40 μl of PBS and 10 μl of plasma at appropriate dilutions were applied. After incubation for 1 h, wells were washed with PBS, and bound mAb1 DANAPA IgG1 or mAb1 IgG1 was detected with 10′000-fold diluted Fc-specific HRP conjugated anti-human IgG1 detection antibody (A0170, Sigma). The assay was developed with QuantaRed fluorogenic substrate (Pierce) and the fluorescence intensity was measured after 2 to 4 min at 544 nm (excitation) and 590 nm (emission). The plasma levels of mAb1 DANAPA IgG1 and mAb1 IgG1 were determined using a standard curve of the respective antibodies. Antibody exposure in the plasma is presented in a semi-logarithmic plot over a period of 216 hours.

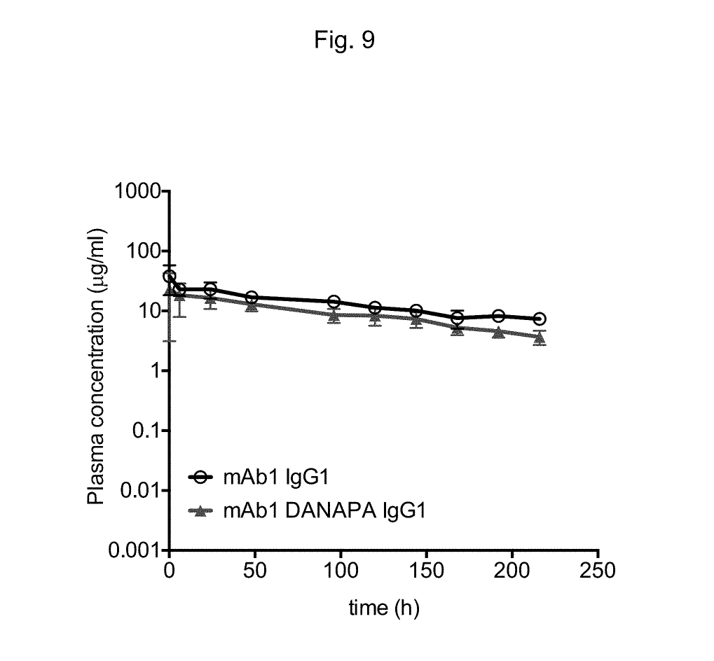

The pharmacokinetic profiles of mAb1 DANAPA IgG1 and of mAb1 IgG1 are shown in FIG. 9. Importantly and unpredictably before the instant invention, mAb1 DANAPA IgG1 has pharmacokinetic properties that are essentially identical to mAb1 IgG1.

### Example 10: CD33×CD3 Bispecific FynomAbs with DANAPA IgG1 Fc Show Reduced FcγR Binding

Upon interaction with Fc receptor expressing cells, Fc-containing bispecific T cell engaging molecules that have a first binding site specific to a tumor antigen and a second binding site specific for T cells may induce tumor-independent T cell activation and cytokine release (off-tumor effect) (see WO2012143524). In order to mitigate this risk, such molecules can be equipped with engineered Fc sequences that have minimal intrinsic FcγR affinity.

CD3/CD33 bispecific FynomAbs were generated by fusion of the CD33-specific Fynomer G1 (SEQ ID NO: 36) or the CD33-specific Fynomer D5 (SEQ ID NO: 38) to the C-terminus of the light chain of a humanized CD3 specific antibody mAb2 (heavy chain SEQ ID NO: 14; light chain SEQ ID NO: 16), using a flexible (G4S)3 linker (SEQ ID NO: 40). FcγR binding of these FynomAbs was determined in an AlphaScreen™ competition assay as described in Example 4.

FcγR binding of the CD3/CD33 FynomAbs was compared to COVA467 (heavy chain SEQ ID NO: 26; light chain SEQ ID NO: 28), a previously described CD3/CD33 FynomAb with a LALA IgG Fc (i.e. an IgG Fc having the L234A and L235A mutations) that was generated by fusing the CD33-specific Fynomer B3 to the C-terminus of the CD3 specific antibody mAb3 light chain (see WO2014170063). B21M IgG1 served as positive control (see Example 4).

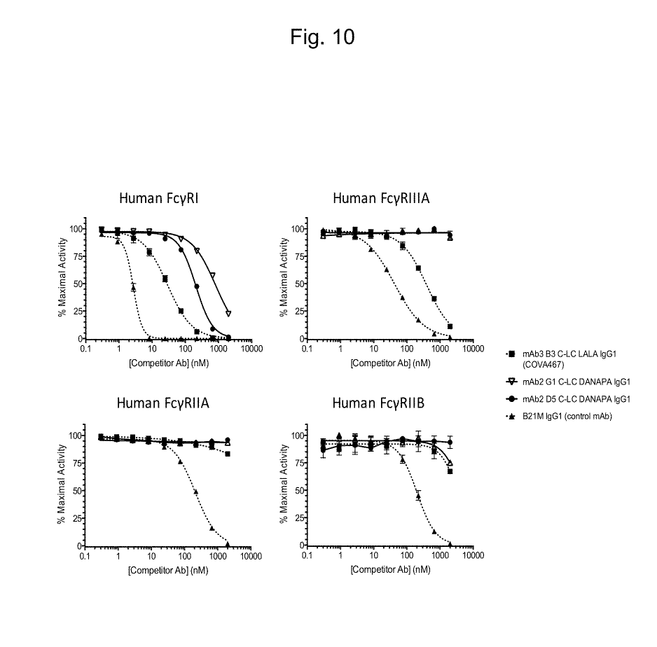

The results are shown in FIG. 10. Whereas COVA467 showed residual binding to human FcγRIIIA and FcγRI (IC₅₀=390 nM and 29 nM, respectively), the two FynomAbs with the DANAPA IgG Fc did not show any binding to FcγRIIIA and strongly reduced binding to FcγRI, compared to COVA467 (IC₅₀=206 nM or 800 nM, respectively). No significant binding to FcγRII A and B was found for the constructs. These results demonstrate that the CD3/CD33 bispecific FynomAbs with a DANAPA IgG Fc show reduced FcγR binding as compared to COVA467. Therefore, they have a reduced potential to induce undesired off-tumor T cell activation and cytokine release and represent improved mutants of CD3/CD33 bispecific FynomAbs.

TABLE 5
Sequences

| SEQ | | |
|---|---|---|
| ID | | |
| NO: | Description | Sequence |
| 1 | mAb1 IgG1 | CAGGTCCAGCTGCAGCAGAGTGGGGCCGAACTGGCAAGACCC |
| | HC | GGAGCAAGCGTCAAAATGTCATGTAAAGCAAGCGGTTATACT |
| | (DNA) | TTCACTAGGAGCACCATGCACTGGGTGAAACAGAGGCCCGGC |
| | | CAGGGACTGGAGTGGATCGGGTACATTAACCCTTCCAGCGCT |
| | | TACACCAACTATAATCAGAAGTTCAAAGACAAGGCCACCCTG |
| | | ACAGCTGATAAGTCTAGTTCAACAGCATATATGCAGCTGTCC |
| | | AGCCTGACTTCTGAAGACAGTGCAGTGTACTATTGCGCCTCC |
| | | CCACAGGTCCACTACGATTACAATGGTTTTCCTTACTGGGGG |
| | | CAGGGCACACTGGTGACTGTCTCCGCCGCTAGCACAAAGGGC |
| | | CCTAGTGTGTTTCCTCTGGCTCCCTCTTCCAAATCCACTTCT |
| | | GGTGGCACTGCTGCTCTGGGATGCCTGGTGAAGGATTACTTT |
| | | CCTGAACCTGTGACTGTCTCATGGAACTCTGGTGCTCTGACT |
| | | TCTGGTGTCCACACTTTCCCTGCTGTGCTGCAGTCTAGTGGA |
| | | CTGTACTCTCTGTCATCTGTGGTCACTGTGCCCTCTTCATCT |
| | | CTGGGAACCCAGACCTACATTTGTAATGTGAACCACAAACCA |
| | | TCCAACACTAAAGTGGACAAAAAAGTGGAACCCAAATCCTGT |
| | | GACAAAACCCACACCTGCCCACCTTGTCCTGCCCCTGAACTG |
| | | CTGGGAGGACCTTCTGTGTTTCTGTTCCCCCCCAAACCAAAG |
| | | GATACCCTGATGATCTCTAGAACCCCTGAGGTGACATGTGTG |
| | | GTGGTGGATGTGTCTCATGAGGACCCTGAGGTCAAATTCAAC |
| | | TGGTACGTGGATGGAGTGGAAGTCCACAATGCCAAAACCAAG |
| | | CCTAGAGAGGAACAGTACAATTCAACCTACAGAGTTGTCAGT |
| | | GTGCTGACTGTGCTGCATCAGGATTGGCTGAATGGCAAGGAA |
| | | TACAAGTGTAAAGTCTCAAACAAGGCCCTGCCTGCTCCAATT |
| | | GAGAAAACAATCTCAAAGGCCAAGGGACAGCCTAGGGAACCC |
| | | CAGGTCTACACCCTGCCACCTTCAAGAGAGGAAATGACCAAA |
| | | AACCAGGTGTCCCTGACATGCCTGGTCAAAGGCTTCTACCCT |
| | | TCTGACATTGCTGTGGAGTGGGAGTCAAATGGACAGCCTGAG |
| | | AACAACTACAAAACAACCCCCCCTGTGCTGGATTCTGATGGC |
| | | TCTTTCTTTCTGTACTCCAAACTGACTGTGGACAAGTCTAGA |
| | | TGGCAGCAGGGGAATGTCTTTTCTTGCTCTGTCATGCATGAG |
| | | GCTCTGCATAACCACTACACTCAGAAATCCCTGTCTCTGTCT |
| | | CCCGGGAAATGA |
| 2 | mAb1 IgG1 | QVQLQQSGAELARPGASVKMSCKASGYTFTRSTMHWVKQRPG |
| | HC | QGLEWIGYINPSSAYTNYNQKFKDKATLTADKSSSTAYMQLS |
| | (protein) | SLTSEDSAVYYCASPQVHYDYNGFPYWGQGTLVTVSAASTKG |
| | | PSVFPLAPSSKSTSGGTAALGCLVKDYFPEPVTVSWNSGALT |
| | | SGVHTFPAVLQSSGLYSLSSVVTVPSSSLGTQTYICNVNHKP |
| | | SNTKVDKKVEPKSCDKTHTCPPCPAPELLGGPSVFLFPPKPK |
| | | DTLMISRTPEVTCVVVDVSHEDPEVKFNWYVDGVEVHNAKTK |
| | | PREEQYNSTYRVVSVLTVLHQDWLNGKEYKCKVSNKALPAPI |
| | | EKTISKAKGQPREPQVYTLPPSREEMTKNQVSLTCLVKGFYP |
| | | SDIAVEWESNGQPENNYKTTPPVLDSDGSFFLYSKLTVDKSR |
| | | WQQGNVFSCSVMHEALHNHYTQKSLSLSPGK |
| 3 | mAb1, mAb1 | CAGGTGGTGCTGACCCAGAGCCCTGCTATTATGTCCGCATTC |
| | DANAPA, | CCCGGTGAAAAAGTGACTATGACTTGTTCCGCTTCTTCCTCC |
| | mAb1 DAPA, | GTCTCCTACATGAACTGGTATCAGCAGAAGTCAGGAACATCT |
| | mAb1 N297A, | CCCAAAAGGTGGATCTACGACTCCAGCAAGCTGGCATCCGGC |
| | mAb1 | GTGCCTGCACGATTCTCAGGCTCCGGAAGCGGGACCTCTTAT |
| | DNNDPG, | AGTCTGACAATTTCTAGTATGGAGACTGAAGATGCCGCTACC |
| | mAb1 | TACTATTGCCAGCAGTGGTCAAGAAACCCTCCAACATTCGGG |
| | DENQPS, and | GGGGGGACTAAACTGCAGATTACTCGTACGGTCGCGGCGCCT |
| | mAb1 LALA- | TCTGTGTTCATTTTCCCCCCATCTGATGAACAGCTGAAATCT |
| | IgG1 LC | GGCACTGCTTCTGTGGTCTGTCTGCTGAACAACTTCTACCCT |
| | (DNA) | AGAGAGGCCAAAGTCCAGTGGAAAGTGGACAATGCTCTGCAG |
| | | AGTGGGAATTCCCAGGAATCTGTCACTGAGCAGGACTCTAAG |
| | | GATAGCACATACTCCCTGTCCTCTACTCTGACACTGAGCAAG |
| | | GCTGATTACGAGAAACACAAAGTGTACGCCTGTGAAGTCACA |
| | | CATCAGGGGCTGTCTAGTCCTGTGACCAAATCCTTCAATAGG |
| | | GGAGAGTGCTGA |
| 4 | mAb1, mAb1 | QVVLTQSPAIMSAFPGEKVTMTCSASSSVSYMNWYQQKSGTS |
| | DANAPA, | PKRWIYDSSKLASGVPARFSGSGSGTSYSLTISSMETEDAAT |
| | mAb1 DAPA, | YYCQQWSRNPPTFGGGTKLQITRTVAAPSVFIFPPSDEQLKS |
| | mAb1 N297A, | GTASVVCLLNNFYPREAKVQWKVDNALQSGNSQESVTEQDSK |
| | mAb1 | DSTYSLSSTLTLSKADYEKHKVYACEVTHQGLSSPVTKSFNR |
| | DNNDPG, | GEC |
| | mAb1 | |
| | DENQPS, and | |
| | mAb1 LALA- | |
| | IgG1 LC | |
| | (protein) | |
| 5 | Anti-PD1 | CAGGTGCAGCTCCAGCAGAGTGGCGCAGAGCTGGTGAAGCCC |
| | mAb DANAPA- | GGAGCCTCAGTCAAGATGTCCTGCAAGGCCTTCGGCTACACT |
| | IgG1 HC | TTTACCACATATCCTATCGAGTGGATGAAGCAGAACCACGGG |
| | (DNA) | AAAAGCCTGGAATGGATTGGTAACTTCCATCCATACAATGAC |
| | | GATACCAAGTATAATGAGAAGTTTAAAGGCAAGGCAAAACTG |
| | | ACAGTGGAGAAATCCAGCACTACCGTCTACCTGGAACTGTCC |
| | | AGGCTGACATCTGACGATAGTGCCGTGTACTATTGTGCTCGG |
| | | GAAAACTACGGAAGCCACGGCGGATTCGTCTATTGGGGGCAG |
| | | GGTACACTGGTGACTGTCTCTGCCGCTAGCACAAAGGGCCCT |
| | | AGTGTGTTTCCTCTGGCTCCCTCTTCCAAATCCACTTCTGGT |
| | | GGCACTGCTGCTCTGGGATGCCTGGTGAAGGATTACTTTCCT |
| | | GAACCTGTGACTGTCTCATGGAACTCTGGTGCTCTGACTTCT |
| | | GGTGTCCACACTTTCCCTGCTGTGCTGCAGTCTAGTGGACTG |
| | | TACTCTCTGTCATCTGTGGTCACTGTGCCCTCTTCATCTCTG |
| | | GGAACCCAGACCTACATTTGTAATGTGAACCACAAACCATCC |
| | | AACACTAAAGTGGACAAAAAAGTGGAACCCAAATCCTGTGAC |
| | | AAAACCCACACCTGCCCACCTTGTCCTGCCCCTGAACTGCTG |
| | | GGAGGACCTTCTGTGTTTCTGTTCCCCCCCAAACCAAAGGAT |
| | | ACCCTGATGATCTCTAGAACCCCTGAGGTGACATGTGTGGTG |
| | | GTGGCTGTGTCTCATGAGGACCCTGAGGTCAAATTCAACTGG |
| | | TACGTGGATGGAGTGGAAGTCCACAATGCCAAAACCAAGCCT |
| | | AGAGAGGAACAGTACGCTTCAACCTACAGAGTTGTCAGTGTG |
| | | CTGACTGTGCTGCATCAGGATTGGCTGAATGGCAAGGAATAC |
| | | AAGTGTAAAGTCTCAAACAAGGCCCTGGCTGCTCCAATTGAG |
| | | AAAACAATCTCAAAGGCCAAGGGACAGCCTAGGGAACCCCAG |
| | | GTCTACACCCTGCCACCTTCAAGAGAGGAAATGACCAAAAAC |
| | | CAGGTGTCCCTGACATGCCTGGTCAAAGGCTTCTACCCTTCT |
| | | GACATTGCTGTGGAGTGGGAGTCAAATGGACAGCCTGAGAAC |
| | | AACTACAAAACAACCCCCCCTGTGCTGGATTCTGATGGCTCT |
| | | TTCTTTCTGTACTCCAAACTGACTGTGGACAAGTCTAGATGG |
| | | CAGCAGGGGAATGTCTTTTCTTGCTCTGTCATGCATGAGGCT |
| 6 | Anti-PD1 | QVQLQQSGAELVKPGASVKMSCKAFGYTFTTYPIEWMKQNHG |
| | mAb DANAPA | KSLEWIGNFHPYNDDTKYNEKFKGKAKLTVEKSSTTVYLELS |
| | IgG1 HC | RLTSDDSAVYYCARENYGSHGGFVYWGQGTLVTVSAASTKGP |
| | (protein) | SVFPLAPSSKSTSGGTAALGCLVKDYFPEPVTVSWNSGALTS |
| | | GVHTFPAVLQSSGLYSLSSVVTVPSSSLGTQTYICNVNHKPS |
| | | NTKVDKKVEPKSCDKTHTCPPCPAPELLGGPSVFLFPPKPKD |
| | | TLMISRTPEVTCVVVAVSHEDPEVKFNWYVDGVEVHNAKTKP |
| | | REEQYASTYRVVSVLTVLHQDWLNGKEYKCKVSNKALAAPIE |
| | | KTISKAKGQPREPQVYTLPPSREEMTKNQVSLTCLVKGFYPS |
| | | DIAVEWESNGQPENNYKTTPPVLDSDGSFFLYSKLTVDKSRW |
| | | QQGNVFSCSVMHEALHNHYTQKSLSLSPGK |
| 7 | Anti-PD1 | GAGAACGTGCTGACCCAGTCCCCCGCAATCATGTCTGCCAGT |
| | mAb DANAPA | CCTGGAGAAAAGGTCACCATGACATGCAGGGCATCCAGCTCT |
| | IgG1 LC | GTCATCAGTTCATACCTGCACTGGTATCAGCAGAAGAGCGGA |
| | (DNA) | GCTTCTCCAAAACTGTGGATCTACTCAACCTCCAACCTGGCA |
| | | AGCGGGGTGCCCGACCGGTTCAGCGGCTCTGGAAGTGGGACT |
| | | TCATATAGTCTGACCATCTCGTCGGTCGAGGCCGAAGATGCC |
| | | GCTACATACTATTGTCAGCAGTACAATGGCTATCCCCTGACA |
| | | TTTGGTGCTGGTACCAAACTCGAGATTAAGCGTACGGTCGCG |
| | | GCGCCTTCTGTGTTCATTTTCCCCCCATCTGATGAACAGCTG |
| | | AAATCTGGCACTGCTTCTGTGGTCTGTCTGCTGAACAACTTC |
| | | TACCCTAGAGAGGCCAAAGTCCAGTGGAAAGTGGACAATGCT |
| | | CTGCAGAGTGGGAATTCCCAGGAATCTGTCACTGAGCAGGAC |
| | | TCTAAGGATAGCACATACTCCCTGTCCTCTACTCTGACACTG |
| | | AGCAAGGCTGATTACGAGAAACACAAAGTGTACGCCTGTGAA |
| | | GTCACACATCAGGGGCTGTCTAGTCCTGTGACCAAATCCTTC |
| | | AATAGGGGAGAGTGCTGA |
| 8 | Anti-PD1 | ENVLTQSPAIMSASPGEKVTMTCRASSSVISSYLHWYQQKSG |
| | mAb DANAPA | ASPKLWIYSTSNLASGVPDRFSGSGSGTSYSLTISSVEAEDA |
| | IgG1 LC | ATYYCQQYNGYPLTFGAGTKLEIKRTVAAPSVFIFPPSDEQL |
| | (protein) | KSGTASVVCLLNNFYPREAKVQWKVDNALQSGNSQESVTEQD |
| | | SKDSTYSLSSTLTLSKADYEKHKVYACEVTHQGLSSPVTKSF |
| | | NRGEC |
| 9 | Anti-HER2 | GAGGTTCAGCTGGTGGAGTCTGGCGGTGGCCTGGTGCAGCCA |
| | mAb DANAPA | GGGGGCTCACTCCGTTTGTCCTGTGCAGCTTCTGGCTTCAAC |
| | IgG1 HC | ATTAAAGACACCTATATACACTGGGTGCGTCAGGCCCCGGGT |
| | (DNA) | AAGGGCCTGGAATGGGTTGCAAGGATTTATCCTACGAATGGT |
| | | TATACTAGATATGCCGATAGCGTCAAGGGCCGTTTCACTATA |
| | | AGCGCAGACACATCCAAAAACACAGCCTACCTGCAGATGAAC |
| | | AGCCTGCGTGCTGAGGACACTGCCGTCTATTATTGTTCTAGA |
| | | TGGGGAGGGGACGGCTTCTATGCTATGGACTACTGGGGTCAA |
| | | GGAACCCTGGTCACCGTCTCCTCGGCTAGCACAAAGGGCCCT |
| | | AGTGTGTTTCCTCTGGCTCCCTCTTCCAAATCCACTTCTGGT |
| | | GGCACTGCTGCTCTGGGATGCCTGGTGAAGGATTACTTTCCT |
| | | GAACCTGTGACTGTCTCATGGAACTCTGGTGCTCTGACTTCT |
| | | GGTGTCCACACTTTCCCTGCTGTGCTGCAGTCTAGTGGACTG |
| | | TACTCTCTGTCATCTGTGGTCACTGTGCCCTCTTCATCTCTG |
| | | GGAACCCAGACCTACATTTGTAATGTGAACCACAAACCATCC |
| | | AACACTAAAGTGGACAAAAAAGTGGAACCCAAATCCTGTGAC |
| | | AAAACCCACACCTGCCCACCTTGTCCTGCCCCTGAACTGCTG |
| | | GGAGGACCTTCTGTGTTTCTGTTCCCCCCCAAACCAAAGGAT |
| | | ACCCTGATGATCTCTAGAACCCCTGAGGTGACATGTGTGGTG |
| | | GTGGCTGTGTCTCATGAGGACCCTGAGGTCAAATTCAACTGG |
| | | TACGTGGATGGAGTGGAAGTCCACAATGCCAAAACCAAGCCT |
| | | AGAGAGGAACAGTACGCTTCAACCTACAGAGTTGTCAGTGTG |
| | | CTGACTGTGCTGCATCAGGATTGGCTGAATGGCAAGGAATAC |
| | | AAGTGTAAAGTCTCAAACAAGGCCCTGGCTGCTCCAATTGAG |
| | | AAAACAATCTCAAAGGCCAAGGGACAGCCTAGGGAACCCCAG |
| | | GTCTACACCCTGCCACCTTCAAGAGAGGAAATGACCAAAAAC |
| | | CAGGTGTCCCTGACATGCCTGGTCAAAGGCTTCTACCCTTCT |
| | | GACATTGCTGTGGAGTGGGAGTCAAATGGACAGCCTGAGAAC |
| | | AACTACAAAACAACCCCCCCTGTGCTGGATTCTGATGGCTCT |
| | | TTCTTTCTGTACTCCAAACTGACTGTGGACAAGTCTAGATGG |
| | | CAGCAGGGGAATGTCTTTTCTTGCTCTGTCATGCATGAGGCT |
| | | CTGCATAACCACTACACTCAGAAATCCCTGTCTCTGTCTCCC |
| | | GGGAAATGA |
| 10 | Anti-HER2 | EVQLVESGGGLVQPGGSLRLSCAASGFNIKDTYIHWVRQAPG |
| | mAb DANAPA | KGLEWVARIYPTNGYTRYADSVKGRFTISADTSKNTAYLQMN |
| | IgG1 HC | SLRAEDTAVYYCSRWGGDGFYAMDYWGQGTLVTVSSASTKGP |
| | (protein) | SVFPLAPSSKSTSGGTAALGCLVKDYFPEPVTVSWNSGALTS |
| | | GVHTFPAVLQSSGLYSLSSVVTVPSSSLGTQTYICNVNHKPS |
| | | NTKVDKKVEPKSCDKTHTCPPCPAPELLGGPSVFLFPPKPKD |
| | | TLMISRTPEVTCVVVAVSHEDPEVKFNWYVDGVEVHNAKTKP |
| | | REEQYASTYRVVSVLTVLHQDWLNGKEYKCKVSNKALAAPIE |
| | | KTISKAKGQPREPQVYTLPPSREEMTKNQVSLTCLVKGFYPS |
| | | DIAVEWESNGQPENNYKTTPPVLDSDGSFFLYSKLTVDKSRW |
| | | QQGNVFSCSVMHEALHNHYTQKSLSLSPGK |
| 11 | Anti-HER2 | GATATCCAGATGACCCAGTCCCCGAGCTCCCTGTCCGCCTCT |
| | mAb DANAPA | GTGGGCGATAGGGTCACCATCACCTGCCGTGCCAGTCAGGAT |
| | IgG1 LC | GTGAATACTGCTGTAGCCTGGTATCAACAGAAACCAGGAAAA |
| | (DNA) | GCTCCGAAACTACTGATTTACTCGGCATCCTTCCTCTACTCT |
| | | GGAGTCCCTTCTCGCTTCTCTGGGTCCAGATCTGGGACGGAT |
| | | TTCACTCTGACCATCAGCAGTCTGCAGCCGGAAGACTTCGCA |
| | | ACTTATTACTGTCAGCAACATTATACTACTCCTCCCACGTTC |
| | | GGACAGGGGACCAAGGTGGAGATCAAACGTACGGTCGCGGCG |
| | | CCTTCTGTGTTCATTTTCCCCCCATCTGATGAACAGCTGAAA |
| | | TCTGGCACTGCTTCTGTGGTCTGTCTGCTGAACAACTTCTAC |
| | | CCTAGAGAGGCCAAAGTCCAGTGGAAAGTGGACAATGCTCTG |
| | | CAGAGTGGGAATTCCCAGGAATCTGTCACTGAGCAGGACTCT |
| | | AAGGATAGCACATACTCCCTGTCCTCTACTCTGACACTGAGC |
| | | AAGGCTGATTACGAGAAACACAAAGTGTACGCCTGTGAAGTC |
| | | ACACATCAGGGGCTGTCTAGTCCTGTGACCAAATCCTTCAAT |
| | | AGGGGAGAGTGCTGA |
| 12 | Anti-HER2 | DIQMTQSPSSLSASVGDRVTITCRASQDVNTAVAWYQQKPGK |
| | mAb DANAPA | APKLLIYSASFLYSGVPSRFSGSRSGTDFTLTISSLQPEDFA |
| | IgG1 LC | TYYCQQHYTTPPTFGQGTKVEIKRTVAAPSVFIFPPSDEQLK |
| | (protein) | SGTASVVCLLNNFYPREAKVQWKVDNALQSGNSQESVTEQDS |
| | | KDSTYSLSSTLTLSKADYEKHKVYACEVTHQGLSSPVTKSFN |
| | | RGEC |
| 13 | mAb2 | GAGGTGCAGCTGGTCGAGTCTGGAGGAGGATTGGTGCAGCCT |
| | DANAPA, | GGAGGGTCATTGAAACTCTCATGTGCAGCCTCTGGATTCACC |
| | mAb2 D5 C- | TTCAATACCTACGCCATGAACTGGGTCCGCCAGGCTCCAGGA |
| | LC DANAPA, | AAGGGTTTGGAATGGGTTGCTCGCATAAGAAGTAAATATAAT |
| | and mAb2 G1 | AATTATGCAACATATTATGCCGATTCAGTGAAAGACAGGTTC |
| | C-LC | ACCATCTCCAGAGATGATTCAAAAAACACTGCCTATCTACAA |
| | DANAPA- | ATGAACAGCTTGAAAACTGAGGACACTGCCGTGTACTACTGT |
| | IgG1 HC | GTGAGACATGGGAACTTCGGTGATAGCTACGTTTCCTGGTTT |
| | (DNA) | GCTTACTGGGGCCAAGGGACTCTGGTCACCGTCTCGAGCGCT |
| | | AGCACAAAGGGCCCTAGTGTGTTTCCTCTGGCTCCCTCTTCC |
| | | AAATCCACTTCTGGTGGCACTGCTGCTCTGGGATGCCTGGTG |
| | | AAGGATTACTTTCCTGAACCTGTGACTGTCTCATGGAACTCT |
| | | GGTGCTCTGACTTCTGGTGTCCACACTTTCCCTGCTGTGCTG |
| | | CAGTCTAGTGGACTGTACTCTCTGTCATCTGTGGTCACTGTG |
| | | CCCTCTTCATCTCTGGGAACCCAGACCTACATTTGTAATGTG |
| | | AACCACAAACCATCCAACACTAAAGTGGACAAAAAAGTGGAA |
| | | CCCAAATCCTGTGACAAAACCCACACCTGCCCACCTTGTCCT |
| | | GCCCCTGAACTGCTGGGAGGACCTTCTGTGTTTCTGTTCCCC |
| | | CCCAAACCAAAGGATACCCTGATGATCTCTAGAACCCCTGAG |
| | | GTGACATGTGTGGTGGTGGCTGTGTCTCATGAGGACCCTGAG |
| | | GTCAAATTCAACTGGTACGTGGATGGAGTGGAAGTCCACAAT |
| | | GCCAAAACCAAGCCTAGAGAGGAACAGTACGCTTCAACCTAC |
| | | AGAGTTGTCAGTGTGCTGACTGTGCTGCATCAGGATTGGCTG |
| | | AATGGCAAGGAATACAAGTGTAAAGTCTCAAACAAGGCCCTG |
| | | GCTGCTCCAATTGAGAAAACAATCTCAAAGGCCAAGGGACAG |
| | | CCTAGGGAACCCCAGGTCTACACCCTGCCACCTTCAAGAGAG |
| | | GAAATGACCAAAAACCAGGTGTCCCTGACATGCCTGGTCAAA |
| | | GGCTTCTACCCTTCTGACATTGCTGTGGAGTGGGAGTCAAAT |
| | | GGACAGCCTGAGAACAACTACAAAACAACCCCCCCTGTGCTG |
| | | GATTCTGATGGCTCTTTCTTTCTGTACTCCAAACTGACTGTG |
| | | GACAAGTCTAGATGGCAGCAGGGGAATGTCTTTTCTTGCTCT |
| | | GTCATGCATGAGGCTCTGCATAACCACTACACTCAGAAATCC |
| | | CTGTCTCTGTCTCCCGGGAAATGA |
| 14 | mAb2 | EVQLVESGGGLVQPGGSLKLSCAASGFTFNTYAMNWVRQAPG |
| | DANAPA, | KGLEWVARIRSKYNNYATYYADSVKDRFTISRDDSKNTAYLQ |
| | mAb2 D5 C- | MNSLKTEDTAVYYCVRHGNFGDSYVSWFAYWGQGTLVTVSSA |
| | LC DANAPA, | STKGPSVFPLAPSSKSTSGGTAALGCLVKDYFPEPVTVSWNS |
| | and mAb2 G1 | GALTSGVHTFPAVLQSSGLYSLSSVVTVPSSSLGTQTYICNV |
| | C-LC | NHKPSNTKVDKKVEPKSCDKTHTCPPCPAPELLGGPSVFLFP |
| | DANAPA- | PKPKDTLMISRTPEVTCVVVAVSHEDPEVKFNWYVDGVEVHN |
| | IgG1 HC | AKTKPREEQYASTYRVVSVLTVLHQDWLNGKEYKCKVSNKAL |
| | (protein) | AAPIEKTISKAKGQPREPQVYTLPPSREEMTKNQVSLTCLVK |
| | | GFYPSDIAVEWESNGQPENNYKTTPPVLDSDGSFFLYSKLTV |
| | | DKSRWQQGNVFSCSVMHEALHNHYTQKSLSLSPGK |
| 15 | mAb2 DANAPA | CAGACCGTTGTGACTCAGGAACCTTCACTCACCGTATCACCT |
| | IgG1 LC | GGTGGAACAGTCACACTCACTTGTCGCTCGTCGACTGGGGCT |
| | (DNA) | GTTACAACTAGCAACTATGCCAACTGGGTCCAACAAAAACCG |
| | | GGTCAGGCACCCCGTGGTCTAATAGGTGGTACCAACAAGCGC |
| | | GCACCAGGTACTCCTGCCAGATTCTCAGGCTCCCTGCTTGGA |
| | | GGCAAGGCTGCCCTCACCCTCTCGGGGGTACAGCCAGAGGAT |
| | | GAGGCAGAATATTACTGTGCTCTATGGTACAGCAACCTCTGG |
| | | GTGTTCGGTGGAGGAACCAAACTGACTGTCCTAGGCCAGCCT |
| | | AAAGCGGCGCCATCCGTCACCCTGTTCCCTCCCTCATCCGAG |
| | | GAACTGCAGGCCAATAAGGCTACACTGGTCTGTCTGATTAGC |
| | | GACTTCTACCCTGGGGCCGTGACTGTGGCTTGGAAAGCCGAT |
| | | TCTTCTCCCGTGAAAGCTGGAGTGGAAACAACCACCCCCTCT |
| | | AAACAGAGCAACAACAAATACGCTGCCTCTTCATACCTGTCC |
| | | CTGACCCCTGAACAGTGGAAATCTCACCGGTCTTACTCATGC |
| | | CAGGTGACACACGAGGGATCAACTGTGGAGAAAACCGTGGCT |
| | | CCTACCGAATGTTCATGA |
| 16 | mAb2 DANAPA | QTVVTQEPSLTVSPGGTVTLTCRSSTGAVTTSNYANWVQQKP |
| | IgG1 LC | GQAPRGLIGGTNKRAPGTPARFSGSLLGGKAALTLSGVQPED |
| | (protein) | EAEYYCALWYSNLWVFGGGTKLTVLGQPKAAPSVTLFPPSSE |
| | | ELQANKATLVCLISDFYPGAVTVAWKADSSPVKAGVETTTPS |
| | | KQSNNKYAASSYLSLTPEQWKSHRSYSCQVTHEGSTVEKTVA |
| | | PTECS |
| 17 | mAb1 DNNDPG | CAGGTCCAGCTGCAGCAGAGTGGGGCCGAACTGGCAAGACCC |
| | IgG1 HC | GGAGCAAGCGTCAAAATGTCATGTAAAGCAAGCGGTTATACT |
| | (DNA) | TTCACTAGGAGCACCATGCACTGGGTGAAACAGAGGCCCGGC |
| | | CAGGGACTGGAGTGGATCGGGTACATTAACCCTTCCAGCGCT |
| | | TACACCAACTATAATCAGAAGTTCAAAGACAAGGCCACCCTG |
| | | ACAGCTGATAAGTCTAGTTCAACAGCATATATGCAGCTGTCC |
| | | AGCCTGACTTCTGAAGACAGTGCAGTGTACTATTGCGCCTCC |
| | | CCACAGGTCCACTACGATTACAATGGTTTTCCTTACTGGGGG |
| | | CAGGGCACACTGGTGACTGTCTCCGCCGCTAGCACAAAGGGC |
| | | CCTAGTGTGTTTCCTCTGGCTCCCTCTTCCAAATCCACTTCT |
| | | GGTGGCACTGCTGCTCTGGGATGCCTGGTGAAGGATTACTTT |
| | | CCTGAACCTGTGACTGTCTCATGGAACTCTGGTGCTCTGACT |
| | | TCTGGTGTCCACACTTTCCCTGCTGTGCTGCAGTCTAGTGGA |
| | | CTGTACTCTCTGTCATCTGTGGTCACTGTGCCCTCTTCATCT |
| | | CTGGGAACCCAGACCTACATTTGTAATGTGAACCACAAACCA |
| | | TCCAACACTAAAGTGGACAAAAAAGTGGAACCCAAATCCTGT |
| | | GACAAAACCCACACCTGCCCACCTTGTCCTGCCCCTGAACTG |
| | | CTGGGAGGACCTTCTGTGTTTCTGTTCCCCCCCAAACCAAAG |
| | | GATACCCTGATGATCTCTAGAACCCCTGAGGTGACATGTGTG |
| | | GTGGTGAATGTGTCTCATGAGGACCCTGAGGTCAAATTCAAC |
| | | TGGTACGTGGATGGAGTGGAAGTCCACAATGCCAAAACCAAG |
| | | CCTAGAGAGGAACAGTACGATTCAACCTACAGAGTTGTCAGT |
| | | GTGCTGACTGTGCTGCATCAGGATTGGCTGAATGGCAAGGAA |
| | | TACAAGTGTAAAGTCTCAAACAAGGCCCTGGGTGCTCCAATT |
| | | GAGAAAACAATCTCAAAGGCCAAGGGACAGCCTAGGGAACCC |
| | | CAGGTCTACACCCTGCCACCTTCAAGAGAGGAAATGACCAAA |
| | | AACCAGGTGTCCCTGACATGCCTGGTCAAAGGCTTCTACCCT |
| | | TCTGACATTGCTGTGGAGTGGGAGTCAAATGGACAGCCTGAG |
| | | AACAACTACAAAACAACCCCCCCTGTGCTGGATTCTGATGGC |
| | | TCTTTCTTTCTGTACTCCAAACTGACTGTGGACAAGTCTAGA |
| | | TGGCAGCAGGGGAATGTCTTTTCTTGCTCTGTCATGCATGAG |
| | | GCTCTGCATAACCACTACACTCAGAAATCCCTGTCTCTGTCT |
| | | CCCGGGAAATGA |
| 18 | mAb1 DNNDPG | QVQLQQSGAELARPGASVKMSCKASGYTFTRSTMHWVKQRPG |
| | IgG1 HC | QGLEWIGYINPSSAYTNYNQKFKDKATLTADKSSSTAYMQLS |
| | (protein) | SLTSEDSAVYYCASPQVHYDYNGFPYWGQGTLVTVSAASTKG |
| | | PSVFPLAPSSKSTSGGTAALGCLVKDYFPEPVTVSWNSGALT |
| | | SGVHTFPAVLQSSGLYSLSSVVTVPSSSLGTQTYICNVNHKP |
| | | SNTKVDKKVEPKSCDKTHTCPPCPAPELLGGPSVFLFPPKPK |
| | | DTLMISRTPEVTCVVVNVSHEDPEVKFNWYVDGVEVHNAKTK |
| | | PREEQYDSTYRVVSVLTVLHQDWLNGKEYKCKVSNKALGAPI |
| | | EKTISKAKGQPREPQVYTLPPSREEMTKNQVSLTCLVKGFYP |
| | | SDIAVEWESNGQPENNYKTTPPVLDSDGSFFLYSKLTVDKSR |
| | | WQQGNVFSCSVMHEALHNHYTQKSLSLSPGK |
| 19 | mAb1 DENQPS | CAGGTCCAGCTGCAGCAGAGTGGGGCCGAACTGGCAAGACCC |
| | IgG1 HC | GGAGCAAGCGTCAAAATGTCATGTAAAGCAAGCGGTTATACT |
| | (DNA) | TTCACTAGGAGCACCATGCACTGGGTGAAACAGAGGCCCGGC |
| | | CAGGGACTGGAGTGGATCGGGTACATTAACCCTTCCAGCGCT |
| | | TACACCAACTATAATCAGAAGTTCAAAGACAAGGCCACCCTG |
| | | ACAGCTGATAAGTCTAGTTCAACAGCATATATGCAGCTGTCC |
| | | AGCCTGACTTCTGAAGACAGTGCAGTGTACTATTGCGCCTCC |
| | | CCACAGGTCCACTACGATTACAATGGTTTTCCTTACTGGGGG |
| | | CAGGGCACACTGGTGACTGTCTCCGCCGCTAGCACAAAGGGC |
| | | CCTAGTGTGTTTCCTCTGGCTCCCTCTTCCAAATCCACTTCT |
| | | GGTGGCACTGCTGCTCTGGGATGCCTGGTGAAGGATTACTTT |
| | | CCTGAACCTGTGACTGTCTCATGGAACTCTGGTGCTCTGACT |
| | | TCTGGTGTCCACACTTTCCCTGCTGTGCTGCAGTCTAGTGGA |
| | | CTGTACTCTCTGTCATCTGTGGTCACTGTGCCCTCTTCATCT |
| | | CTGGGAACCCAGACCTACATTTGTAATGTGAACCACAAACCA |
| | | TCCAACACTAAAGTGGACAAAAAAGTGGAACCCAAATCCTGT |
| | | GACAAAACCCACACCTGCCCACCTTGTCCTGCCCCTGAACTG |
| | | CTGGGAGGACCTTCTGTGTTTCTGTTCCCCCCCAAACCAAAG |
| | | GATACCCTGATGATCTCTAGAACCCCTGAGGTGACATGTGTG |
| | | GTGGTGGAGGTGTCTCATGAGGACCCTGAGGTCAAATTCAAC |
| | | TGGTACGTGGATGGAGTGGAAGTCCACAATGCCAAAACCAAG |
| | | CCTAGAGAGGAACAGTACCAATCAACCTACAGAGTTGTCAGT |
| | | GTGCTGACTGTGCTGCATCAGGATTGGCTGAATGGCAAGGAA |
| | | TACAAGTGTAAAGTCTCAAACAAGGCCCTGTCTGCTCCAATT |
| | | GAGAAAACAATCTCAAAGGCCAAGGGACAGCCTAGGGAACCC |
| | | CAGGTCTACACCCTGCCACCTTCAAGAGAGGAAATGACCAAA |
| | | AACCAGGTGTCCCTGACATGCCTGGTCAAAGGCTTCTACCCT |
| | | TCTGACATTGCTGTGGAGTGGGAGTCAAATGGACAGCCTGAG |
| | | AACAACTACAAAACAACCCCCCCTGTGCTGGATTCTGATGGC |
| | | TCTTTCTTTCTGTACTCCAAACTGACTGTGGACAAGTCTAGA |
| | | TGGCAGCAGGGGAATGTCTTTTCTTGCTCTGTCATGCATGAG |
| | | GCTCTGCATAACCACTACACTCAGAAATCCCTGTCTCTGTCT |
| | | CCCGGGAAATGA |
| 20 | mAb1 DENQPS | QVQLQQSGAELARPGASVKMSCKASGYTFTRSTMHWVKQRPG |
| | IgG1 HC | QGLEWIGYINPSSAYTNYNQKFKDKATLTADKSSSTAYMQLS |
| | (protein) | SLTSEDSAVYYCASPQVHYDYNGFPYWGQGTLVTVSAASTKG |
| | | PSVFPLAPSSKSTSGGTAALGCLVKDYFPEPVTVSWNSGALT |
| | | SGVHTFPAVLQSSGLYSLSSVVTVPSSSLGTQTYICNVNHKP |
| | | SNTKVDKKVEPKSCDKTHTCPPCPAPELLGGPSVFLFPPKPK |
| | | DTLMISRTPEVTCVVVEVSHEDPEVKFNWYVDGVEVHNAKTK |
| | | PREEQYQSTYRVVSVLTVLHQDWLNGKEYKCKVSNKALSAPI |
| | | EKTISKAKGQPREPQVYTLPPSREEMTKNQVSLTCLVKGFYP |
| | | SDIAVEWESNGQPENNYKTTPPVLDSDGSFFLYSKLTVDKSR |
| | | WQQGNVFSCSVMHEALHNHYTQKSLSLSPGK |
| 21 | mAb2 G1 C- | CAGACCGTTGTGACTCAGGAACCTTCACTCACCGTATCACCT |
| | LC DANAPA | GGTGGAACAGTCACACTCACTTGTCGCTCGTCGACTGGGGCT |
| | IgG1 LC | GTTACAACTAGCAACTATGCCAACTGGGTCCAACAAAAACCG |
| | (DNA) | GGTCAGGCACCCCGTGGTCTAATAGGTGGTACCAACAAGCGC |
| | | GCACCAGGTACTCCTGCCAGATTCTCAGGCTCCCTGCTTGGA |
| | | GGCAAGGCTGCCCTCACCCTCTCGGGGGTACAGCCAGAGGAT |
| | | GAGGCAGAATATTACTGTGCTCTATGGTACAGCAACCTCTGG |
| | | GTGTTCGGTGGAGGAACCAAACTGACTGTCCTAGGCCAGCCT |
| | | AAAGCGGCGCCATCCGTCACCCTGTTCCCTCCCTCATCCGAG |
| | | GAACTGCAGGCCAATAAGGCTACACTGGTCTGTCTGATTAGC |
| | | GACTTCTACCCTGGGGCCGTGACTGTGGCTTGGAAAGCCGAT |
| | | TCTTCTCCCGTGAAAGCTGGAGTGGAAACAACCACCCCCTCT |
| | | AAACAGAGCAACAACAAATACGCTGCCTCTTCATACCTGTCC |
| | | CTGACCCCTGAACAGTGGAAATCTCACCGGTCTTACTCATGC |
| | | CAGGTGACACACGAGGGATCAACTGTGGAGAAAACCGTGGCT |
| | | CCTACCGAATGTTCAGGCGGTGGAGGATCCGGGGGTGGGGGA |
| | | AGCGGCGGAGGAGGTAGCGGCGTGACTCTGTTCGTCGCTCTG |
| | | TACGACTATGAGGCCCTGGGGGCTCACGAACTGTCCTTCCAT |
| | | AAGGGCGAGAAATTTCAGATCCTGTCCCCCAGGAGCGAGGGA |
| | | CCTTTTTGGGAAGCACACTCTCTGACCACAGGCGAAACCGGA |
| | | TGGATTCCCTCTAACTACGTGGCCCCCGTCGATAGTATTCAG |
| | | TGA |
| 22 | mAb2 G1 C- | QTVVTQEPSLTVSPGGTVTLTCRSSTGAVTTSNYANWVQQKP |
| | LC DANAPA | GQAPRGLIGGTNKRAPGTPARFSGSLLGGKAALTLSGVQPED |
| | IgG1 LC | EAEYYCALWYSNLWVFGGGTKLTVLGQPKAAPSVTLFPPSSE |
| | (protein) | ELQANKATLVCLISDFYPGAVTVAWKADSSPVKAGVETTTPS |
| | | KQSNNKYAASSYLSLTPEQWKSHRSYSCQVTHEGSTVEKTVA |
| | | PTECSGGGGSGGGGSGGGGSGVTLFVALYDYEALGAHELSFH |
| | | KGEKFQILSPRSEGPFWEAHSLTTGETGWIPSNYVAPVDSIQ |
| 23 | mAb2 D5 C- | CAGACCGTTGTGACTCAGGAACCTTCACTCACCGTATCACCT |
| | LC DANAPA | GGTGGAACAGTCACACTCACTTGTCGCTCGTCGACTGGGGCT |
| | IgG1 LC | GTTACAACTAGCAACTATGCCAACTGGGTCCAACAAAAACCG |
| | (DNA) | GGTCAGGCACCCCGTGGTCTAATAGGTGGTACCAACAAGCGC |
| | | GCACCAGGTACTCCTGCCAGATTCTCAGGCTCCCTGCTTGGA |
| | | GGCAAGGCTGCCCTCACCCTCTCGGGGGTACAGCCAGAGGAT |
| | | GAGGCAGAATATTACTGTGCTCTATGGTACAGCAACCTCTGG |
| | | GTGTTCGGTGGAGGAACCAAACTGACTGTCCTAGGCCAGCCT |
| | | AAAGCGGCGCCATCCGTCACCCTGTTCCCTCCCTCATCCGAG |
| | | GAACTGCAGGCCAATAAGGCTACACTGGTCTGTCTGATTAGC |
| | | GACTTCTACCCTGGGGCCGTGACTGTGGCTTGGAAAGCCGAT |
| | | TCTTCTCCCGTGAAAGCTGGAGTGGAAACAACCACCCCCTCT |
| | | AAACAGAGCAACAACAAATACGCTGCCTCTTCATACCTGTCC |
| | | CTGACCCCTGAACAGTGGAAATCTCACCGGTCTTACTCATGC |
| | | CAGGTGACACACGAGGGATCAACTGTGGAGAAAACCGTGGCT |
| | | CCTACCGAATGTTCAGGCGGTGGAGGATCCGGGGGTGGGGGA |
| | | AGCGGCGGAGGAGGTAGCGGCGTGACTCTGTTCGTCGCTCTG |
| | | TACGACTATGAGGCCCTGGGGGCTCACGAACTGTCCTTCCAT |
| | | AAGGGCGAGAAATTTCAGATCCTGTCCAGCCTGGCAGTGGGA |
| | | CCATTTTGGGAGGCCCACTCTCTGACCACAGGCGAAACCGGA |
| | | TGGATTCCCTCTAACTACGTGGCACCTGTCGATAGTATTCAG |
| | | TGA |
| 24 | mAb2 D5 C- | QTVVTQEPSLTVSPGGTVTLTCRSSTGAVTTSNYANWVQQKP |
| | LC DANAPA | GQAPRGLIGGTNKRAPGTPARFSGSLLGGKAALTLSGVQPED |
| | IgG1 LC | EAEYYCALWYSNLWVFGGGTKLTVLGQPKAAPSVTLFPPSSE |
| | (protein) | ELQANKATLVCLISDFYPGAVTVAWKADSSPVKAGVETTTPS |
| | | KQSNNKYAASSYLSLTPEQWKSHRSYSCQVTHEGSTVEKTVA |
| | | PTECSGGGGSGGGGSGGGGSGVTLFVALYDYEALGAHELSFH |
| | | KGEKFQILSSLAVGPFWEAHSLTTGETGWIPSNYVAPVDSIQ |
| 25 | mAb3 B3 C- | CAGGTGCAGCTGGTGCAGTCTGGCGGCGGAGTGGTGCAGCCT |
| | LC LALA | GGAAGATCCCTGCGGCTGTCCTGCAAGGCCTCCGGCTACACC |
| | IgG1 | TTCACCCGGTACACCATGCACTGGGTGCGACAGGCCCCTGGC |
| | (COVA467) | AAGGGCCTGGAATGGATCGGCTACATCAACCCCTCCCGGGGC |
| | HC | TACACCAACTACAACCAGAAAGTGAAGGACCGGTTCACCATC |
| | (DNA) | TCCCGGGACAACTCCAAGAACACCGCCTTTCTGCAGATGGAC |
| | | AGCCTGCGGCCTGAGGATACCGGCGTGTACTTCTGCGCCCGG |
| | | TACTACGACGACCACTACTGCCTGGACTACTGGGGCCAGGGC |
| | | ACCCCTGTGACAGTGTCCTCTGCTAGCACAAAGGGCCCTAGT |
| | | GTGTTTCCTCTGGCTCCCTCTTCCAAATCCACTTCTGGTGGC |
| | | ACTGCTGCTCTGGGATGCCTGGTGAAGGATTACTTTCCTGAA |
| | | CCTGTGACTGTCTCATGGAACTCTGGTGCTCTGACTTCTGGT |
| | | GTCCACACTTTCCCTGCTGTGCTGCAGTCTAGTGGACTGTAC |
| | | TCTCTGTCATCTGTGGTCACTGTGCCCTCTTCATCTCTGGGA |
| | | ACCCAGACCTACATTTGTAATGTGAACCACAAACCATCCAAC |
| | | ACTAAAGTGGACAAAAAAGTGGAACCCAAATCCTGTGACAAA |
| | | ACCCACACCTGCCCACCTTGTCCTGCCCCTGAAGCCGCCGGA |
| | | GGACCTTCTGTGTTTCTGTTCCCCCCCAAACCAAAGGATACC |
| | | CTGATGATCTCTAGAACCCCTGAGGTGACATGTGTGGTGGTG |
| | | GATGTGTCTCATGAGGACCCTGAGGTCAAATTCAACTGGTAC |
| | | GTGGATGGAGTGGAAGTCCACAATGCCAAAACCAAGCCTAGA |
| | | GAGGAACAGTACAATTCAACCTACAGAGTGGTCAGTGTGCTG |
| | | ACTGTGCTGCATCAGGATTGGCTGAATGGCAAGGAATACAAG |
| | | TGTAAAGTCTCAAACAAGGCCCTGCCTGCTCCAATTGAGAAA |
| | | ACAATCTCAAAGGCCAAGGGACAGCCTAGGGAACCCCAGGTC |
| | | TACACCCTGCCACCTTCAAGAGAGGAAATGACCAAAAACCAG |
| | | GTGTCCCTGACATGCCTGGTCAAAGGCTTCTACCCTTCTGAC |
| | | ATTGCTGTGGAGTGGGAGTCAAATGGACAGCCTGAGAACAAC |
| | | TACAAAACAACCCCCCCTGTGCTGGATTCTGATGGCTCTTTC |
| | | TTTCTGTACTCCAAACTGACTGTGGACAAGTCTAGATGGCAG |
| | | CAGGGGAATGTCTTTTCTTGCTCTGTCATGCATGAGGCTCTG |
| | | CATAACCACTACACTCAGAAATCCCTGTCTCTGTCTCCCGGG |
| | | AATGA |
| 26 | mAb3 B3 C- | QVQLVQSGGGVVQPGRSLRLSCKASGYTFTRYTMHWVRQAPG |
| | LC LALA | KGLEWIGYINPSRGYTNYNQKVKDRFTISRDNSKNTAFLQMD |
| | IgG1 | SLRPEDTGVYFCARYYDDHYCLDYWGQGTPVTVSSASTKGPS |
| | (COVA467) | VFPLAPSSKSTSGGTAALGCLVKDYFPEPVTVSWNSGALTSG |
| | HC | VHTFPAVLQSSGLYSLSSVVTVPSSSLGTQTYICNVNHKPSN |
| | (protein) | TKVDKKVEPKSCDKTHTCPPCPAPEAAGGPSVFLFPPKPKDT |
| | | LMISRTPEVTCVVVDVSHEDPEVKFNWYVDGVEVHNAKTKPR |
| | | EEQYNSTYRVVSVLTVLHQDWLNGKEYKCKVSNKALPAPIEK |
| | | TISKAKGQPREPQVYTLPPSREEMTKNQVSLTCLVKGFYPSD |
| | | IAVEWESNGQPENNYKTTPPVLDSDGSFFLYSKLTVDKSRWQ |
| | | QGNVFSCSVMHEALHNHYTQKSLSLSPGK |
| 27 | mAb3 B3 C- | GACATCCAGATGACCCAGTCCCCCTCCAGCCTGTCTGCCTCT |
| | LC LALA | GTGGGCGACAGAGTGACAATTACCTGCTCCGCCTCCTCCTCC |
| | IgG1 | GTGTCCTACATGAACTGGTATCAGCAGACCCCCGGCAAGGCC |
| | (COVA467) | CCCAAGCGGTGGATCTACGACACCTCCAAGCTGGCCTCTGGC |
| | LC | GTGCCCTCCAGATTCTCCGGCTCTGGCTCTGGCACCGACTAT |
| | (DNA) | ACCTTCACCATCAGCTCCCTGCAGCCCGAGGATATCGCCACC |
| | | TACTACTGCCAGCAGTGGTCCTCCAACCCCTTCACCTTTGGC |
| | | CAGGGCACCAAGCTGCAGATCACCCGTACGGTCGCGGCGCCT |
| | | TCTGTGTTCATTTTCCCCCCATCTGATGAACAGCTGAAATCT |
| | | GGCACTGCTTCTGTGGTCTGTCTGCTGAACAACTTCTACCCT |
| | | AGAGAGGCCAAAGTCCAGTGGAAAGTGGACAATGCTCTGCAG |
| | | AGTGGGAATTCCCAGGAATCTGTCACTGAGCAGGACTCTAAG |
| | | GATAGCACATACTCCCTGTCCTCTACTCTGACACTGAGCAAG |
| | | GCTGATTACGAGAAACACAAAGTGTACGCCTGTGAAGTCACA |
| | | CATCAGGGGCTGTCTAGTCCTGTGACCAAATCCTTCAATAGG |
| | | GGAGAGTGCGGCGGTGGAGGATCCGGGGGTGGGGGAAGCGGC |
| | | GGAGGAGGTAGCGGCGTGACCCTGTTTGTGGCCCTGTACGAC |
| | | TACGAGGCCCTGGGCGCTCACGAGCTGTCTTTCCACAAGGGC |
| | | GAGAAGTTCCAGATCCTGAACTCCTCCGAGGGCCCCTTCTGG |
| | | GAGGCTCACTCTCTGACAACCGGCGAGACAGGCTGGATTCCC |
| | | TCCAACTATGTGGCCCCCGTGGACTCCATCCAGTGA |
| 28 | mAb3 B3 C- | DIQMTQSPSSLSASVGDRVTITCSASSSVSYMNWYQQTPGKA |
| | LC LALA | PKRWIYDTSKLASGVPSRFSGSGSGTDYTFTISSLQPEDIAT |
| | IgG1 | YYCQQWSSNPFTFGQGTKLQITRTVAAPSVFIFPPSDEQLKS |
| | (COVA467) | GTASVVCLLNNFYPREAKVQWKVDNALQSGNSQESVTEQDSK |
| | LC | DSTYSLSSTLTLSKADYEKHKVYACEVTHQGLSSPVTKSFNR |
| | (protein) | GECGGGGSGGGGSGGGGSGVTLFVALYDYEALGAHELSFHKG |
| | | EKFQILNSSEGPFWEAHSLTTGETGWIPSNYVAPVDSIQ |
| 29 | B21M IgG1 | CAGATCACCCTGAAGGAGTCCGGGCCCACACTGGTGAAACCT |
| | HC | ACTCAGACCCTGACACTGACTTGCACCTTCTCCGGTTTTTCT |
| | (DNA) | CTGAGTACCTCGGGCATGGGAGTGAGCTGGATCAGGCAGCCC |
| | | CCTGGCAAGGCACTGGAATGGCTGGCCCACATCTACTGGGAC |
| | | GATGACAAGAGGTACAACCCTTCACTGAAATCCCGGCTGACA |
| | | ATTACTAAGGATACCAGCAAAAACCAGGTGGTCCTGACCATG |
| | | ACAAATATGGACCCCGTGGACACTGCTACCTACTATTGTGCA |
| | | AGACTGTACGGCTTCACCTATGGATTTGCTTACTGGGGGCAG |
| | | GGCACCCTGGTCACAGTCTCGAGCGCTAGCACAAAGGGCCCT |
| | | AGTGTGTTTCCTCTGGCTCCCTCTTCCAAATCCACTTCTGGT |
| | | GGCACTGCTGCTCTGGGATGCCTGGTGAAGGATTACTTTCCT |
| | | GAACCTGTGACTGTCTCATGGAACTCTGGTGCTCTGACTTCT |
| | | GGTGTCCACACTTTCCCTGCTGTGCTGCAGTCTAGTGGACTG |
| | | TACTCTCTGTCATCTGTGGTCACTGTGCCCTCTTCATCTCTG |
| | | GGAACCCAGACCTACATTTGTAATGTGAACCACAAACCATCC |
| | | AACACTAAAGTGGACAAAAAAGTGGAACCCAAATCCTGTGAC |
| | | AAAACCCACACCTGCCCACCTTGTCCTGCCCCTGAACTGCTG |
| | | GGAGGACCTTCTGTGTTTCTGTTCCCCCCCAAACCAAAGGAT |
| | | ACCCTGATGATCTCTAGAACCCCTGAGGTGACATGTGTGGTG |
| | | GTGGATGTGTCTCATGAGGACCCTGAGGTCAAATTCAACTGG |
| | | TACGTGGATGGAGTGGAAGTCCACAATGCCAAAACCAAGCCT |
| | | AGAGAGGAACAGTACAATTCAACCTACAGAGTTGTCAGTGTG |
| | | CTGACTGTGCTGCATCAGGATTGGCTGAATGGCAAGGAATAC |
| | | AAGTGTAAAGTCTCAAACAAGGCCCTGCCTGCTCCAATTGAG |
| | | AAAACAATCTCAAAGGCCAAGGGACAGCCTAGGGAACCCCAG |
| | | GTCTACACCCTGCCACCTTCAAGAGAGGAAATGACCAAAAAC |
| | | CAGGTGTCCCTGACATGCCTGGTCAAAGGCTTCTACCCTTCT |
| | | GACATTGCTGTGGAGTGGGAGTCAAATGGACAGCCTGAGAAC |
| | | AACTACAAAACAACCCCCCCTGTGCTGGATTCTGATGGCTCT |
| | | TTCTTTCTGTACTCCAAACTGACTGTGGACAAGTCTAGATGG |
| | | CAGCAGGGGAATGTCTTTTCTTGCTCTGTCATGCATGAGGCT |
| | | CTGCATAACCACTACACTCAGAAATCCCTGTCTCTGTCTCCC |
| | | GGGAAATGA |
| 30 | B21M IgG1 | QITLKESGPTLVKPTQTLTLTCTFSGFSLSTSGMGVSWIRQP |
| | HC | PGKALEWLAHIYWDDDKRYNPSLKSRLTITKDTSKNQVVLTM |
| | (protein) | TNMDPVDTATYYCARLYGFTYGFAYWGQGTLVTVSSASTKGP |
| | | SVFPLAPSSKSTSGGTAALGCLVKDYFPEPVTVSWNSGALTS |
| | | GVHTFPAVLQSSGLYSLSSVVTVPSSSLGTQTYICNVNHKPS |
| | | NTKVDKKVEPKSCDKTHTCPPCPAPELLGGPSVFLFPPKPKD |
| | | TLMISRTPEVTCVVVDVSHEDPEVKFNWYVDGVEVHNAKTKP |
| | | REEQYNSTYRVVSVLTVLHQDWLNGKEYKCKVSNKALPAPIE |
| | | KTISKAKGQPREPQVYTLPPSREEMTKNQVSLTCLVKGFYPS |
| | | DIAVEWESNGQPENNYKTTPPVLDSDGSFFLYSKLTVDKSRW |
| | | QQGNVFSCSVMHEALHNHYTQKSLSLSPGK |
| 31 | B21M IgG1 | GACATCGTGATGACACAGAGCCCAGATTCTCTGGCCGTCAGC |
| | LC | CTGGGCGAAAGGGCCACTATCAACTGCCGGGCCTCCCAGTCT |
| | (DNA) | GTGGACTACAATGGAATTTCTTACATGCACTGGTATCAGCAG |
| | | AAGCCTGGCCAGCCCCCTAAACTGCTGATCTATGCCGCTTCA |
| | | AACCCTGAGTCCGGCGTGCCAGACCGATTCAGTGGCTCAGGC |
| | | TCCGGGACCGATTTTACCCTGACAATTTCCAGCCTGCAAGCT |
| | | GAGGACGTGGCAGTCTACTATTGCCAGCAGATCATTGAAGAT |
| | | CCCTGGACATTCGGTCAGGGCACTAAGGTGGAGATCAAACGT |
| | | ACGGTCGCGGCGCCTTCTGTGTTCATTTTCCCCCCATCTGAT |
| | | GAACAGCTGAAATCTGGCACTGCTTCTGTGGTCTGTCTGCTG |
| | | AACAACTTCTACCCTAGAGAGGCCAAAGTCCAGTGGAAAGTG |
| | | GACAATGCTCTGCAGAGTGGGAATTCCCAGGAATCTGTCACT |
| | | GAGCAGGACTCTAAGGATAGCACATACTCCCTGTCCTCTACT |
| | | CTGACACTGAGCAAGGCTGATTACGAGAAACACAAAGTGTAC |
| | | GCCTGTGAAGTCACACATCAGGGGCTGTCTAGTCCTGTGACC |
| | | AAATCCTTCAATAGGGGAGAGTGCTGA |
| 32 | B21M and | DIVMTQSPDSLAVSLGERATINCRASQSVDYNGISYMHWYQQ |
| | B21M LALA- | KPGQPPKLLIYAASNPESGVPDRFSGSGSGTDFTLTISSLQA |
| | IgG1 LC | EDVAVYYCQQIIEDPWTFGQGTKVEIKRTVAAPSVFIFPPSD |
| | (protein) | EQLKSGTASVVCLLNNFYPREAKVQWKVDNALQSGNSQESVT |
| | | EQDSKDSTYSLSSTLTLSKADYEKHKVYACEVTHQGLSSPVT |
| | | KSFNRGEC |
| 33 | B21M LALA | CAGATCACCCTGAAGGAGTCCGGGCCCACACTGGTGAAACCT |
| | IgG1 HC | ACTCAGACCCTGACACTGACTTGCACCTTCTCCGGTTTTTCT |
| | (DNA) | CTGAGTACCTCGGGCATGGGAGTGAGCTGGATCAGGCAGCCC |
| | | CCTGGCAAGGCACTGGAATGGCTGGCCCACATCTACTGGGAC |
| | | GATGACAAGAGGTACAACCCTTCACTGAAATCCCGGCTGACA |
| | | ATTACTAAGGATACCAGCAAAAACCAGGTGGTCCTGACCATG |
| | | ACAAATATGGACCCCGTGGACACTGCTACCTACTATTGTGCA |
| | | AGACTGTACGGCTTCACCTATGGATTTGCTTACTGGGGGCAG |
| | | GGCACCCTGGTCACAGTCTCGAGCGCTAGCACAAAGGGCCCT |
| | | AGTGTGTTTCCTCTGGCTCCCTCTTCCAAATCCACTTCTGGT |
| | | GGCACTGCTGCTCTGGGATGCCTGGTGAAGGATTACTTTCCT |
| | | GAACCTGTGACTGTCTCATGGAACTCTGGTGCTCTGACTTCT |
| | | GGTGTCCACACTTTCCCTGCTGTGCTGCAGTCTAGTGGACTG |
| | | TACTCTCTGTCATCTGTGGTCACTGTGCCCTCTTCATCTCTG |
| | | GGAACCCAGACCTACATTTGTAATGTGAACCACAAACCATCC |
| | | AACACTAAAGTGGACAAAAAAGTGGAACCCAAATCCTGTGAC |
| | | AAAACCCACACCTGCCCACCTTGTCCTGCCCCTGAAGCCGCC |
| | | GGAGGACCTTCTGTGTTTCTGTTCCCCCCCAAACCAAAGGAT |
| | | ACCCTGATGATCTCTAGAACCCCTGAGGTGACATGTGTGGTG |
| | | GTGGATGTGTCTCATGAGGACCCTGAGGTCAAATTCAACTGG |
| | | TACGTGGATGGAGTGGAAGTCCACAATGCCAAAACCAAGCCT |
| | | AGAGAGGAACAGTACAATTCAACCTACAGAGTGGTCAGTGTG |
| | | CTGACTGTGCTGCATCAGGATTGGCTGAATGGCAAGGAATAC |
| | | AAGTGTAAAGTCTCAAACAAGGCCCTGCCTGCTCCAATTGAG |
| | | AAAACAATCTCAAAGGCCAAGGGACAGCCTAGGGAACCCCAG |
| | | GTCTACACCCTGCCACCTTCAAGAGAGGAAATGACCAAAAAC |
| | | CAGGTGTCCCTGACATGCCTGGTCAAAGGCTTCTACCCTTCT |
| | | GACATTGCTGTGGAGTGGGAGTCAAATGGACAGCCTGAGAAC |
| | | AACTACAAAACAACCCCCCCTGTGCTGGATTCTGATGGCTCT |
| | | TTCTTTCTGTACTCCAAACTGACTGTGGACAAGTCTAGATGG |
| | | CAGCAGGGGAATGTCTTTTCTTGCTCTGTCATGCATGAGGCT |
| | | CTGCATAACCACTACACTCAGAAATCCCTGTCTCTGTCTCCC |
| | | GGGAAATGA |
| 34 | B21M LALA | QITLKESGPTLVKPTQTLTLTCTFSGFSLSTSGMGVSWIRQP |
| | IgG1 HC | PGKALEWLAHIYWDDDKRYNPSLKSRLTITKDTSKNQVVLTM |
| | (protein) | TNMDPVDTATYYCARLYGFTYGFAYWGQGTLVTVSSASTKGP |
| | | SVFPLAPSSKSTSGGTAALGCLVKDYFPEPVTVSWNSGALTS |
| | | GVHTFPAVLQSSGLYSLSSVVTVPSSSLGTQTYICNVNHKPS |
| | | NTKVDKKVEPKSCDKTHTCPPCPAPEAAGGPSVFLFPPKPKD |
| | | TLMISRTPEVTCVVVDVSHEDPEVKFNWYVDGVEVHNAKTKP |
| | | REEQYNSTYRVVSVLTVLHQDWLNGKEYKCKVSNKALPAPIE |
| | | KTISKAKGQPREPQVYTLPPSREEMTKNQVSLTCLVKGFYPS |
| | | DIAVEWESNGQPENNYKTTPPVLDSDGSFFLYSKLTVDKSRW |
| | | QQGNVFSCSVMHEALHNHYTQKSLSLSPGK |
| 35 | Fynomer G1 | GGCGTGACTCTGTTCGTCGCTCTGTACGACTATGAGGCCCTG |
| | (DNA) | GGGGCTCACGAACTGTCCTTCCATAAGGGCGAGAAATTTCAG |
| | | ATCCTGTCCCCCAGGAGCGAGGGACCTTTTTGGGAAGCACAC |
| | | TCTCTGACCACAGGCGAAACCGGATGGATTCCCTCTAACTAC |
| | | GTGGCCCCCGTCGATAGTATTCAGTGA |
| 36 | Fynomer G1 | GVTLFVALYDYEALGAHELSFHKGEKFQILSPRSEGPFWEAH |
| | (protein) | SLTTGETGWIPSNYVAPVDSIQ |
| 37 | Fynomer D5 | GGCGTGACTCTGTTCGTCGCTCTGTACGACTATGAGGCCCTG |
| | (DNA) | GGGGCTCACGAACTGTCCTTCCATAAGGGCGAGAAATTTCAG |
| | | ATCCTGTCCAGCCTGGCAGTGGGACCATTTTGGGAGGCCCAC |
| | | TCTCTGACCACAGGCGAAACCGGATGGATTCCCTCTAACTAC |
| | | GTGGCACCTGTCGATAGTATTCAGTGA |
| 38 | Fynomer D5 | GVTLFVALYDYEALGAHELSFHKGEKFQILSSLAVGPFWEAH |
| | (protein) | SLTTGETGWIPSNYVAPVDSIQ |
| 39 | (G4S)3 | GGCGGTGGAGGATCCGGGGGTGGGGGAAGCGGCGGAGGAGGT |
| | linker | AGC |
| | (DNA) | |
| 40 | (G4S)3 | GGGGSGGGGSGGGGS |
| | linker | |
| | (protein) | |
| 41 | Leader | ATGAATTTTGGACTGAGGCTGATTTTCCTGGTGCTGACCCTG |
| | sequence | AAAGGCGTCCAGTGT |
| | (DNA) | |
| 42 | Leader | MNFGLRLIFLVLTLKGVQC |
| | Sequence | |
| | (protein) | |
| 43 | Wild-type | CPPCPAPELLGGPSVFLFPPKPKDTLMISRTPEVTCVVVDVS |
| | human IgG1 | HEDPEVKFNWYVDGVEVHNAKTKPREEQYNSTYRVVSVLTVL |
| | Fc | HQDWLNGKEYKCKVSNKALPAPIEKTISKAKGQPREPQVYTL |
| | (starting | PPSREEMTKNQVSLTCLVKGFYPSDIAVEWESNGQPENNYKT |
| | at C226, | TPPVLDSDGSFFLYSKLTVDKSRWQQGNVFSCSVMHEALHNH |
| | according | YTQKSLSLSPGK |
| | to Kabat | |
| | numbering, | |
| | positions | |
| | 265, 297 | |
| | and 329 in | |
| | underlined | |
| | italics) | |
| | (protein) | |
| 44 | mAb1 DANAPA | CAGGTCCAGCTGCAGCAGAGTGGGGCCGAACTGGCAAGACCC |
| | IgG1 HC | GGAGCAAGCGTCAAAATGTCATGTAAAGCAAGCGGTTATACT |
| | (DNA) | TTCACTAGGAGCACCATGCACTGGGTGAAACAGAGGCCCGGC |
| | | CAGGGACTGGAGTGGATCGGGTACATTAACCCTTCCAGCGCT |
| | | TACACCAACTATAATCAGAAGTTCAAAGACAAGGCCACCCTG |
| | | ACAGCTGATAAGTCTAGTTCAACAGCATATATGCAGCTGTCC |
| | | AGCCTGACTTCTGAAGACAGTGCAGTGTACTATTGCGCCTCC |
| | | CCACAGGTCCACTACGATTACAATGGTTTTCCTTACTGGGGG |
| | | CAGGGCACACTGGTGACTGTCTCCGCCGCTAGCACAAAGGGC |
| | | CCTAGTGTGTTTCCTCTGGCTCCCTCTTCCAAATCCACTTCT |
| | | GGTGGCACTGCTGCTCTGGGATGCCTGGTGAAGGATTACTTT |
| | | CCTGAACCTGTGACTGTCTCATGGAACTCTGGTGCTCTGACT |
| | | TCTGGTGTCCACACTTTCCCTGCTGTGCTGCAGTCTAGTGGA |
| | | CTGTACTCTCTGTCATCTGTGGTCACTGTGCCCTCTTCATCT |
| | | CTGGGAACCCAGACCTACATTTGTAATGTGAACCACAAACCA |
| | | TCCAACACTAAAGTGGACAAAAAAGTGGAACCCAAATCCTGT |
| | | GACAAAACCCACACCTGCCCACCTTGTCCTGCCCCTGAACTG |
| | | CTGGGAGGACCTTCTGTGTTTCTGTTCCCCCCCAAACCAAAG |
| | | GATACCCTGATGATCTCTAGAACCCCTGAGGTGACATGTGTG |
| | | GTGGTGGCTGTGTCTCATGAGGACCCTGAGGTCAAATTCAAC |
| | | TGGTACGTGGATGGAGTGGAAGTCCACAATGCCAAAACCAAG |
| | | CCTAGAGAGGAACAGTACGCTTCAACCTACAGAGTTGTCAGT |
| | | GTGCTGACTGTGCTGCATCAGGATTGGCTGAATGGCAAGGAA |
| | | TACAAGTGTAAAGTCTCAAACAAGGCCCTGGCTGCTCCAATT |
| | | GAGAAAACAATCTCAAAGGCCAAGGGACAGCCTAGGGAACCC |
| | | CAGGTCTACACCCTGCCACCTTCAAGAGAGGAAATGACCAAA |
| | | AACCAGGTGTCCCTGACATGCCTGGTCAAAGGCTTCTACCCT |
| | | TCTGACATTGCTGTGGAGTGGGAGTCAAATGGACAGCCTGAG |
| | | AACAACTACAAAACAACCCCCCCTGTGCTGGATTCTGATGGC |
| | | TCTTTCTTTCTGTACTCCAAACTGACTGTGGACAAGTCTAGA |
| | | TGGCAGCAGGGGAATGTCTTTTCTTGCTCTGTCATGCATGAG |
| | | GCTCTGCATAACCACTACACTCAGAAATCCCTGTCTCTGTCT |
| | | CCCGGGAAATGA |
| 45 | mAb1 DANAPA | QVQLQQSGAELARPGASVKMSCKASGYTFTRSTMHWVKQRPG |
| | IgG1 HC | QGLEWIGYINPSSAYTNYNQKFKDKATLTADKSSSTAYMQLS |
| | (protein) | SLTSEDSAVYYCASPQVHYDYNGFPYWGQGTLVTVSAASTKG |
| | | PSVFPLAPSSKSTSGGTAALGCLVKDYFPEPVTVSWNSGALT |
| | | SGVHTFPAVLQSSGLYSLSSVVTVPSSSLGTQTYICNVNHKP |
| | | SNTKVDKKVEPKSCDKTHTCPPCPAPELLGGPSVFLFPPKPK |
| | | DTLMISRTPEVTCVVVAVSHEDPEVKFNWYVDGVEVHNAKTK |
| | | PREEQYASTYRVVSVLTVLHQDWLNGKEYKCKVSNKALAAPI |
| | | EKTISKAKGQPREPQVYTLPPSREEMTKNQVSLTCLVKGFYP |
| | | SDIAVEWESNGQPENNYKTTPPVLDSDGSFFLYSKLTVDKSR |
| | | WQQGNVFSCSVMHEALHNHYTQKSLSLSPGK |
| 46 | mAb1 DAPA | CAGGTCCAGCTGCAGCAGAGTGGGGCCGAACTGGCAAGACCC |
| | IgG1 HC | GGAGCAAGCGTCAAAATGTCATGTAAAGCAAGCGGTTATACT |
| | (DNA) | TTCACTAGGAGCACCATGCACTGGGTGAAACAGAGGCCCGGC |
| | | CAGGGACTGGAGTGGATCGGGTACATTAACCCTTCCAGCGCT |
| | | TACACCAACTATAATCAGAAGTTCAAAGACAAGGCCACCCTG |
| | | ACAGCTGATAAGTCTAGTTCAACAGCATATATGCAGCTGTCC |
| | | AGCCTGACTTCTGAAGACAGTGCAGTGTACTATTGCGCCTCC |
| | | CCACAGGTCCACTACGATTACAATGGTTTTCCTTACTGGGGG |
| | | CAGGGCACACTGGTGACTGTCTCCGCCGCTAGCACAAAGGGC |
| | | CCTAGTGTGTTTCCTCTGGCTCCCTCTTCCAAATCCACTTCT |
| | | GGTGGCACTGCTGCTCTGGGATGCCTGGTGAAGGATTACTTT |
| | | CCTGAACCTGTGACTGTCTCATGGAACTCTGGTGCTCTGACT |
| | | TCTGGTGTCCACACTTTCCCTGCTGTGCTGCAGTCTAGTGGA |
| | | CTGTACTCTCTGTCATCTGTGGTCACTGTGCCCTCTTCATCT |
| | | CTGGGAACCCAGACCTACATTTGTAATGTGAACCACAAACCA |
| | | TCCAACACTAAAGTGGACAAAAAAGTGGAACCCAAATCCTGT |
| | | GACAAAACCCACACCTGCCCACCTTGTCCTGCCCCTGAACTG |
| | | CTGGGAGGACCTTCTGTGTTTCTGTTCCCCCCCAAACCAAAG |
| | | GATACCCTGATGATCTCTAGAACCCCTGAGGTGACATGTGTG |
| | | GTGGTGGCTGTGTCTCATGAGGACCCTGAGGTCAAATTCAAC |
| | | TGGTACGTGGATGGAGTGGAAGTCCACAATGCCAAAACCAAG |
| | | CCTAGAGAGGAACAGTACAATTCAACCTACAGAGTTGTCAGT |
| | | GTGCTGACTGTGCTGCATCAGGATTGGCTGAATGGCAAGGAA |
| | | TACAAGTGTAAAGTCTCAAACAAGGCCCTGGCTGCTCCAATT |
| | | GAGAAAACAATCTCAAAGGCCAAGGGACAGCCTAGGGAACCC |
| | | CAGGTCTACACCCTGCCACCTTCAAGAGAGGAAATGACCAAA |
| | | AACCAGGTGTCCCTGACATGCCTGGTCAAAGGCTTCTACCCT |
| | | TCTGACATTGCTGTGGAGTGGGAGTCAAATGGACAGCCTGAG |
| | | AACAACTACAAAACAACCCCCCCTGTGCTGGATTCTGATGGC |
| | | TCTTTCTTTCTGTACTCCAAACTGACTGTGGACAAGTCTAGA |
| | | TGGCAGCAGGGGAATGTCTTTTCTTGCTCTGTCATGCATGAG |
| | | GCTCTGCATAACCACTACACTCAGAAATCCCTGTCTCTGTCT |
| | | CCCGGGAAATGA |
| 47 | mAb1 DAPA | QVQLQQSGAELARPGASVKMSCKASGYTFTRSTMHWVKQRPG |
| | IgG1 HC | QGLEWIGYINPSSAYTNYNQKFKDKATLTADKSSSTAYMQLS |
| | (protein) | SLTSEDSAVYYCASPQVHYDYNGFPYWGQGTLVTVSAASTKG |
| | | PSVFPLAPSSKSTSGGTAALGCLVKDYFPEPVTVSWNSGALT |
| | | SGVHTFPAVLQSSGLYSLSSVVTVPSSSLGTQTYICNVNHKP |
| | | SNTKVDKKVEPKSCDKTHTCPPCPAPELLGGPSVFLFPPKPK |
| | | DTLMISRTPEVTCVVVAVSHEDPEVKFNWYVDGVEVHNAKTK |
| | | PREEQYNSTYRVVSVLTVLHQDWLNGKEYKCKVSNKALAAPI |
| | | EKTISKAKGQPREPQVYTLPPSREEMTKNQVSLTCLVKGFYP |
| | | SDIAVEWESNGQPENNYKTTPPVLDSDGSFFLYSKLTVDKSR |
| | | WQQGNVFSCSVMHEALHNHYTQKSLSLSPGK |
| 48 | mAb1 N297A | CAGGTCCAGCTGCAGCAGAGTGGGGCCGAACTGGCAAGACCC |
| | IgG1 HC | GGAGCAAGCGTCAAAATGTCATGTAAAGCAAGCGGTTATACT |
| | (DNA) | TTCACTAGGAGCACCATGCACTGGGTGAAACAGAGGCCCGGC |
| | | CAGGGACTGGAGTGGATCGGGTACATTAACCCTTCCAGCGCT |
| | | TACACCAACTATAATCAGAAGTTCAAAGACAAGGCCACCCTG |
| | | ACAGCTGATAAGTCTAGTTCAACAGCATATATGCAGCTGTCC |
| | | AGCCTGACTTCTGAAGACAGTGCAGTGTACTATTGCGCCTCC |
| | | CCACAGGTCCACTACGATTACAATGGTTTTCCTTACTGGGGG |
| | | CAGGGCACACTGGTGACTGTCTCCGCCGCTAGCACAAAGGGC |
| | | CCTAGTGTGTTTCCTCTGGCTCCCTCTTCCAAATCCACTTCT |
| | | GGTGGCACTGCTGCTCTGGGATGCCTGGTGAAGGATTACTTT |
| | | CCTGAACCTGTGACTGTCTCATGGAACTCTGGTGCTCTGACT |
| | | TCTGGTGTCCACACTTTCCCTGCTGTGCTGCAGTCTAGTGGA |
| | | CTGTACTCTCTGTCATCTGTGGTCACTGTGCCCTCTTCATCT |
| | | CTGGGAACCCAGACCTACATTTGTAATGTGAACCACAAACCA |
| | | TCCAACACTAAAGTGGACAAAAAAGTGGAACCCAAATCCTGT |
| | | GACAAAACCCACACCTGCCCACCTTGTCCTGCCCCTGAACTG |
| | | CTGGGAGGACCTTCTGTGTTTCTGTTCCCCCCCAAACCAAAG |
| | | GATACCCTGATGATCTCTAGAACCCCTGAGGTGACATGTGTG |
| | | GTGGTGGATGTGTCTCATGAGGACCCTGAGGTCAAATTCAAC |
| | | TGGTACGTGGATGGAGTGGAAGTCCACAATGCCAAAACCAAG |
| | | CCTAGAGAGGAACAGTACGCTTCAACCTACAGAGTTGTCAGT |
| | | GTGCTGACTGTGCTGCATCAGGATTGGCTGAATGGCAAGGAA |
| | | TACAAGTGTAAAGTCTCAAACAAGGCCCTGCCTGCTCCAATT |
| | | GAGAAAACAATCTCAAAGGCCAAGGGACAGCCTAGGGAACCC |
| | | CAGGTCTACACCCTGCCACCTTCAAGAGAGGAAATGACCAAA |
| | | AACCAGGTGTCCCTGACATGCCTGGTCAAAGGCTTCTACCCT |
| | | TCTGACATTGCTGTGGAGTGGGAGTCAAATGGACAGCCTGAG |
| | | AACAACTACAAAACAACCCCCCCTGTGCTGGATTCTGATGGC |
| | | TCTTTCTTTCTGTACTCCAAACTGACTGTGGACAAGTCTAGA |
| | | TGGCAGCAGGGGAATGTCTTTTCTTGCTCTGTCATGCATGAG |
| | | GCTCTGCATAACCACTACACTCAGAAATCCCTGTCTCTGTCT |
| | | CCCGGGAAATGA |
| 49 | mAb1 N297A | QVQLQQSGAELARPGASVKMSCKASGYTFTRSTMHWVKQRPG |
| | IgG1 HC | QGLEWIGYINPSSAYTNYNQKFKDKATLTADKSSSTAYMQLS |
| | (protein) | SLTSEDSAVYYCASPQVHYDYNGFPYWGQGTLVTVSAASTKG |
| | | PSVFPLAPSSKSTSGGTAALGCLVKDYFPEPVTVSWNSGALT |
| | | SGVHTFPAVLQSSGLYSLSSVVTVPSSSLGTQTYICNVNHKP |
| | | SNTKVDKKVEPKSCDKTHTCPPCPAPELLGGPSVFLFPPKPK |
| | | DTLMISRTPEVTCVVVDVSHEDPEVKFNWYVDGVEVHNAKTK |
| | | PREEQYASTYRVVSVLTVLHQDWLNGKEYKCKVSNKALPAPI |
| | | EKTISKAKGQPREPQVYTLPPSREEMTKNQVSLTCLVKGFYP |
| | | SDIAVEWESNGQPENNYKTTPPVLDSDGSFFLYSKLTVDKSR |
| | | WQQGNVFSCSVMHEALHNHYTQKSLSLSPGK |
| 50 | mAb1 LALA | CAGGTCCAGCTGCAGCAGAGTGGGGCCGAACTGGCAAGACCC |
| | IgG1 HC | GGAGCAAGCGTCAAAATGTCATGTAAAGCAAGCGGTTATACT |
| | (DNA) | TTCACTAGGAGCACCATGCACTGGGTGAAACAGAGGCCCGGC |
| | | CAGGGACTGGAGTGGATCGGGTACATTAACCCTTCCAGCGCT |
| | | TACACCAACTATAATCAGAAGTTCAAAGACAAGGCCACCCTG |
| | | ACAGCTGATAAGTCTAGTTCAACAGCATATATGCAGCTGTCC |
| | | AGCCTGACTTCTGAAGACAGTGCAGTGTACTATTGCGCCTCC |
| | | CCACAGGTCCACTACGATTACAATGGTTTTCCTTACTGGGGG |
| | | CAGGGCACACTGGTGACTGTCTCCGCCGCTAGCACAAAGGGC |
| | | CCTAGTGTGTTTCCTCTGGCTCCCTCTTCCAAATCCACTTCT |
| | | GGTGGCACTGCTGCTCTGGGATGCCTGGTGAAGGATTACTTT |
| | | CCTGAACCTGTGACTGTCTCATGGAACTCTGGTGCTCTGACT |
| | | TCTGGTGTCCACACTTTCCCTGCTGTGCTGCAGTCTAGTGGA |
| | | CTGTACTCTCTGTCATCTGTGGTCACTGTGCCCTCTTCATCT |
| | | CTGGGAACCCAGACCTACATTTGTAATGTGAACCACAAACCA |
| | | TCCAACACTAAAGTGGACAAAAAAGTGGAACCCAAATCCTGT |
| | | GACAAAACCCACACCTGCCCACCTTGTCCTGCCCCTGAAGCC |
| | | GCCGGAGGACCTTCTGTGTTTCTGTTCCCCCCCAAACCAAAG |
| | | GATACCCTGATGATCTCTAGAACCCCTGAGGTGACATGTGTG |
| | | GTGGTGGATGTGTCTCATGAGGACCCTGAGGTCAAATTCAAC |
| | | TGGTACGTGGATGGAGTGGAAGTCCACAATGCCAAAACCAAG |
| | | CCTAGAGAGGAACAGTACAATTCAACCTACAGAGTGGTCAGT |
| | | GTGCTGACTGTGCTGCATCAGGATTGGCTGAATGGCAAGGAA |
| | | TACAAGTGTAAAGTCTCAAACAAGGCCCTGCCTGCTCCAATT |
| | | GAGAAAACAATCTCAAAGGCCAAGGGACAGCCTAGGGAACCC |
| | | CAGGTCTACACCCTGCCACCTTCAAGAGAGGAAATGACCAAA |
| | | AACCAGGTGTCCCTGACATGCCTGGTCAAAGGCTTCTACCCT |
| | | TCTGACATTGCTGTGGAGTGGGAGTCAAATGGACAGCCTGAG |
| | | AACAACTACAAAACAACCCCCCCTGTGCTGGATTCTGATGGC |
| | | TCTTTCTTTCTGTACTCCAAACTGACTGTGGACAAGTCTAGA |
| | | TGGCAGCAGGGGAATGTCTTTTCTTGCTCTGTCATGCATGAG |
| | | GCTCTGCATAACCACTACACTCAGAAATCCCTGTCTCTGTCT |
| | | CCCGGGAAATGA |
| 51 | mAb1 LALA | QVQLQQSGAELARPGASVKMSCKASGYTFTRSTMHWVKQRPG |
| | IgG1 HC | QGLEWIGYINPSSAYTNYNQKFKDKATLTADKSSSTAYMQLS |
| | (protein) | SLTSEDSAVYYCASPQVHYDYNGFPYWGQGTLVTVSAASTKG |
| | | PSVFPLAPSSKSTSGGTAALGCLVKDYFPEPVTVSWNSGALT |
| | | SGVHTFPAVLQSSGLYSLSSVVTVPSSSLGTQTYICNVNHKP |
| | | SNTKVDKKVEPKSCDKTHTCPPCPAPEAAGGPSVFLFPPKPK |
| | | DTLMISRTPEVTCVVVDVSHEDPEVKFNWYVDGVEVHNAKTK |
| | | PREEQYNSTYRVVSVLTVLHQDWLNGKEYKCKVSNKALPAPI |
| | | EKTISKAKGQPREPQVYTLPPSREEMTKNQVSLTCLVKGFYP |
| | | SDIAVEWESNGQPENNYKTTPPVLDSDGSFFLYSKLTVDKSR |
| | | WQQGNVFSCSVMHEALHNHYTQKSLSLSPGK |
| 52 | Wild-type | CPPCPAPELLGGPSVFLFPPKPKDTLMISRTPEVTCVVVDVS |
| | human IgG1 | HEDPEVKFNWYVDGVEVHNAKTKPREEQYNSTYRVVSVLTVL |
| | Fc (allelic | HQDWLNGKEYKCKVSNKALPAPIEKTISKAKGQPREPQVYTL |
| | variant | PPSRDEMTKNQVSLTCLVKGFYPSDIAVEWESNGQPENNYKT |
| | 356D, Kabat | TPPVLDSDGSFFLYSKLTVDKSRWQQGNVFSCSVMHEALHNH |
| | numbering, | YTQKSLSLSPGK |
| | positions | |
| | 265, 297 | |
| | and 329 in | |
| | underlined | |
| | italics) | |
| | (protein) | |
| 53 | Wild-type | CPPCPAPELLGGPSVFLFPPKPKDTLMISRTPEVTCVVVDVS |
| | human IgG1 | HEDPEVKFNWYVDGVEVHNAKTKPREEQYNSTYRVVSVLTVL |
| | Fc (allelic | HQDWLNGKEYKCKVSNKALPAPIEKTISKAKGQPREPQVYTL |
| | variant | PPSREELTKNQVSLTCLVKGFYPSDIAVEWESNGQPENNYKT |
| | 358L, Kabat | TPPVLDSDGSFFLYSKLTVDKSRWQQGNVFSCSVMHEALHNH |
| | numbering, | YTQKSLSLSPGK |
| | positions | |
| | 265, 297 | |
| | and 329 in | |
| | underlined | |
| | italics) | |
| | (protein) | |
| 54 | Wild-type | CPPCPAPELLGGPSVFLFPPKPKDTLMISRTPEVTCVVVDVS |
| | human IgG1 | HEDPEVKFNWYVDGVEVHNAKTKPREEQYNSTYRVVSVLTVL |
| | Fc (allelic | HQDWLNGKEYKCKVSNKALPAPIEKTISKAKGQPREPQVYTL |
| | variant | PPSREEMTKNQVSLTCLVKGFYPSDIAVEWESNGQPENNYKT |
| | 431G, Kabat | TPPVLDSDGSFFLYSKLTVDKSRWQQGNVFSCSVMHEGLHNH |
| | numbering, | YTQKSLSLSPGK |
| | positions | |
| | 265, 297 | |
| | and 329 in | |
| | underlined | |
| | italics) | |
| | (protein) | |
| 55 | Wild-type | CPPCPAPELLGGPSVFLFPPKPKDTLMISRTPEVTCVVVDVS |
| | human IgG1 | HEDPEVKFNWYVDGVEVHNAKTKPREEQYNSTYRVVSVLTVL |
| | Fc (allelic | HQDWLNGKEYKCKVSNKALPAPIEKTISKAKGQPREPQVYTL |
| | variant | PPSRDELTKNQVSLTCLVKGFYPSDIAVEWESNGQPENNYKT |
| | 356D, 358L, | TPPVLDSDGSFFLYSKLTVDKSRWQQGNVFSCSVMHEALHNH |
| | Kabat | YTQKSLSLSPGK |
| | numbering, | |
| | positions | |
| | 265, 297 | |
| | and 329 in | |
| | underlined | |
| | italics) | |
| | (protein) | |
| 56 | Wild-type | CPPCPAPELLGGPSVFLFPPKPKDTLMISRTPEVTCVVVDVS |
| | human IgG1 | HEDPEVKFNWYVDGVEVHNAKTKPREEQYNSTYRVVSVLTVL |
| | Fc (allelic | HQDWLNGKEYKCKVSNKALPAPIEKTISKAKGQPREPQVYTL |
| | variant | PPSRDEMTKNQVSLTCLVKGFYPSDIAVEWESNGQPENNYKT |
| | 356D, 431G, | TPPVLDSDGSFFLYSKLTVDKSRWQQGNVFSCSVMHEGLHNH |
| | Kabat | YTQKSLSLSPGK |
| | numbering, | |
| | positions | |
| | 265, 297 | |
| | and 329 in | |
| | underlined | |
| | italics) | |
| | (protein) | |
| 57 | Wild-type | CPPCPAPELLGGPSVFLFPPKPKDTLMISRTPEVTCVVVDVS |
| | human IgG1 | HEDPEVKFNWYVDGVEVHNAKTKPREEQYNSTYRVVSVLTVL |
| | Fc (allelic | HQDWLNGKEYKCKVSNKALPAPIEKTISKAKGQPREPQVYTL |
| | variant | PPSREELTKNQVSLTCLVKGFYPSDIAVEWESNGQPENNYKT |
| | 358L, 431G, | TPPVLDSDGSFFLYSKLTVDKSRWQQGNVFSCSVMHEGLHNH |
| | Kabat | YTQKSLSLSPGK |
| | numbering, | |
| | positions | |
| | 265, 297 | |
| | and 329 in | |
| | underlined | |
| | italics) | |
| | (protein) | |
| 58 | Wild-type | CPPCPAPELLGGPSVFLFPPKPKDTLMISRTPEVTCVVVDVS |
| | human IgG1 | HEDPEVKFNWYVDGVEVHNAKTKPREEQYNSTYRVVSVLTVL |
| | Fc (allelic | HQDWLNGKEYKCKVSNKALPAPIEKTISKAKGQPREPQVYTL |
| | variant | PPSRDELTKNQVSLTCLVKGFYPSDIAVEWESNGQPENNYKT |
| | 356D, 358L, | TPPVLDSDGSFFLYSKLTVDKSRWQQGNVFSCSVMHEGLHNH |
| | 431G, Kabat | YTQKSLSLSPGK |
| | numbering, | |
| | positions | |
| | 265, 297 | |
| | and 329 in | |
| | underlined | |
| | italics) | |
| | (protein) | |
| 59 | Fyn SH3 | GVTLFVALYDYEARTEDDLSFHKGEKFQILNSSEGDWWEARS |
| | domain | LTTGETGYIPSNYVAPVDSIQ |
| | (protein) | |
| 60 | wild-type | APELLGGPSVFLFPPKPKDTLMISRTPEVTCVVVDVSHEDPE |
| | human IgG1 | VKFNWYVDGVEVHNAKTKPREEQYNSTYRVVSVLTVLHQDWL |
| | CH2 domain | NGKEYKCKVSNKALPAPIEKTISKAK |
| 61 | wild-type | GQPREPQVYTLPPSRDELTKNQVSLTCLVKGFYPSDIAVEWE |
| | human IgG1 | SNGQPENNYKTTPPVLDSDGSFFLYSKLTVDKSRWQQGNVFS |
| | CH3 domain | CSVMHEALHNHYTQKSLSLSPGK |

1. A recombinant IgG Fc-containing molecule, comprising a CH2 domain in which the amino acid at position 265 is different from aspartic acid (D), the amino acid at position 297 is different from asparagine (N), and the amino acid at position 329 is different from proline (P), wherein
the molecule has reduced binding to C1q and to at least one Fcγ receptor (FcγR), as compared to a wild-type IgG1 Fc-containing molecule that comprises D at position 265, N at position 297 and P at position 329,
and wherein the numbering is indicated by the EU index as in Kabat.

2. The molecule of claim 1, wherein the molecule retains binding to FcRn.

3. The molecule of claim 2, wherein at least one FcγR is FcγRI, FcγRIIa, FcγRIIb, FcγRIIIa, and FcγRIIIb.

4. The molecule of claim 3, wherein
i. the amino acid at position 265 is alanine (A), asparagine (N) or glutamic acid (E),
ii. the amino acid at position 297 is alanine (A), aspartic acid (D), or glutamine (Q), and
iii. the amino acid at position 329 is replaced with alanine (A), glycine (G), or serine (S).

5. The molecule of claim 4, wherein the CH2 domain comprises an amino acid sequence that is at least 80%, preferably at least 90% identical to the amino acid sequence of SEQ ID NO: 60.

6. The molecule of claim 4, wherein the Fc domain comprises an amino acid sequence that is at least 80%, preferably at least 90% identical to the amino acid sequence of the human IgG1 Fc domain comprising SEQ ID NO: 43.

7. The molecule of claim 1, wherein the molecule is an antibody, an Fc region, an Fc-fusion protein, or antibody fusion protein such as a FynomAb.

8. The molecule of claim 4, wherein the molecule comprises an Fc region comprising a sequence according to any one of SEQ ID NOs: 43, 52, 53, 54, 55, 56, 57, or 58, wherein amino acids D at position 265, N at position 297 and P at position 329 are replaced by other amino acids.

9. A recombinant polynucleotide encoding the molecule of claim 8.

10. A vector comprising the polynucleotide of claim 9.

11. A host cell comprising the recombinant polynucleotide of claim 9 or the vector of claim 10.

12. A method of making a recombinant IgG1 Fc-containing molecule, comprising a CH2 domain in which amino acids at position 265, 297, and 329 indicated by the EU index as in Kabat are replaced by other amino acids, the method comprising the steps of:
a. providing a nucleic acid encoding a wild-type IgG1 Fc-containing molecule,
b. modifying the nucleic acid provided in step (a) so as to obtain a nucleic acid encoding a recombinant IgG1 Fc-containing molecule wherein the amino acids at position 265, 297, and 329 are replaced with amino acids other than D, N and P, respectively, and
c. expressing the nucleic acid obtaining in step (b) in a host cell and recovering the said mutant.

13. A recombinant polypeptide comprising
a. at least one binding domain capable of binding a target molecule; and
b. an IgG1 Fc domain wherein the amino acids at positions 265, 297, and 329 according to the EU index as in Kabat are different from D, N, and P, respectively,
wherein the polypeptide is capable of binding the target molecule without triggering significant lymphocyte activation, complement dependent lysis, and/or cell mediated destruction of the target molecule and/or of a cell that expresses the target molecule on its surface.

14. The recombinant polypeptide of claim 13, wherein the at least one binding domain is selected from the group consisting of a binding site of an antibody, a Fynomer, an enzyme, a hormone, an extracellular domain of a receptor, a cytokine, an immune cell surface antigen, a ligand, and an adhesion molecule.

15. The recombinant polypeptide of claim 13, wherein the Fc domain is at least 80%, preferably at least 90% identical to the amino acid sequence of the human IgG1 Fc domain comprising SEQ ID NO: 43.

16. The recombinant polypeptide of claim 15 wherein the binding domain is the binding site of an antibody.

17. A pharmaceutical composition comprising the IgG1 Fc-containing molecule of any one of claim 8, the recombinant polynucleotide of claim 9, the vector of claim 10, or the recombinant polypeptide of claim 15 and a pharmaceutically acceptable excipient.

18. A method of treating disease or disorder, comprising administering to a subject or patient the IgG1 Fc-containing molecule of claim 8, the recombinant polynucleotide of claim 9, the vector of claim 10, the recombinant polypeptide of any one of claim 15, or the pharmaceutical composition according to claim 17.

19. The method of claim 18, wherein the disease or disorder is cancer.

20. A method for producing a recombinant IgG1 Fc-containing molecule, the method comprising expressing the recombinant polynucleotide of claim 9 in a host cell and harvesting the the recombinant polypeptide.
